SEQUENCE LISTING (121 sequences)

SEQ ID NO: 1 - DNA, 68571 bp, Homo sapiens
aagcttttactgaatttcagtgggtcattctagagacttggtagccacatgcgtgtcagtttgttgatattcagtattgattattctcataggagctgtatataattgttttgagcttaaaacaaagaaaatgattagtttatggttctaatcttttggatttttttttttttttttagacggtgtttcacttttgtcgcccaggctggaatgcagtggcgcgatctcggcttactgcaacctccacctcccgggttcaagcgattctccatcctcagcctcctgagtagctgggattacaggcgcccgccaccacaccaggctaatttttgtatttttagaagagacagggtttcaccacattggccaggctggttttgaactcctgacctcaggtgatccacccccctcggcctcccaaagtgctgggattacaggcgtgagccaccacgcctggccttggatgttttaaagtaaaattttccccactgcaaaataatagatatgtgttgtaggaacttttggaaacacaaggcacaaaggaaaaaataccttcatatttctccttttcccatctccccagagacttttttctccttatttgttggcatatgtctttccatactcttcaatgcatatatattcacaggataacattatatgtggtattttagaaccacatttctaacattccatttatcatgttttgtatttgcatagtattccatttcgtagttgtaccataatatctttagacaatgcttgtggtcaaataatttcccccaatattttactgttacaaaaaatgtttcgatgaacattttggtctaaagttgatggatcaaggaatataggggtcattataagttaacaaatacagcgtcagatcatggtactactctgctgaaaatcccccttggctcccatcttactcagagccaaagctaaggtctttaccatgacctaaaataccctcagccatccggagtgtgcatgtatgcatacacacagacatgtacatgccactcactcctgcctctctagctcatcttctcctgctcacccctgacttgttcccctgaagctattcctggaatacaccaggcaagcaagcccctgtgtcagggccttggcacttcttccttctgcctataacaattcctccaagtatccacaggcttcctctttcacttcctttgttgcgttggctcagatatcgccttatctgagtgcccttccttaactatacaatacaaaataagttcatgaatctgccctcacccctacattccctgtttcctttaccttgttctatttttctataacatttacctgacaaatcttatgtttgtttattgtcactcttcttccagaatgtaagcactgtgaagtcaaggactttgttagttttgatcactattgtatccatgggttctaaagcactgcctggcacatagttggcactcagtaagctaagaaataataagactaacatattgggattgtggcaaggaggtaagtatactccactttttcaaggggattataaagggcatcatttttaggtgggagatattggacataggagtttgtgaaccaagagacagagaaagatgttacttggagatatggaggttataagtatcagaagattagaagttccggggatgttttaaagtaaaatttttcccattgcaaaataatagatatctgttgtaggaacgtttagaaatacaactaaagcactttttgctttacttggagtgggtgggactggaattgggaggaggggccatggagaggctgctttttttcattgtgcatctttctgtaagacgtgaatttataccatgtacaccaattacttaaataataagagcatgtttggacttccagtttctggtgatggctggttatgaaaacttgaagaaacaaaataatggctttttttaagtgaagaactaaaaaaacattgaacttccaggccagtttttggataaaagtctataaccagggagggaaatggaatactggaacaaaaagctgcctgtgctgagacagcagttgctgataccacaaacacttgtctgctgtttctgtggcctggctgagccaagaaaacatgttaaagcacagagcccacgcaagacagtgtgcttgcaggagaccctaccaacaggaatcggataacaaggagctaaaggtaaggaggagtcagagctgaacccagtgccctcttcccagcagttgtaatgaactgtggctaccttggtgctcttggagcagggaggaaaagaaatagaaaatacaatttccctggaaggcctgtgcatatctgcttccctgaagattagcttctcaaatatacatagctggttgggccaaggattctcaaaatctgaattcggcagaagcacagcaagatctctctggaagagggctcatctcaagcgtcaggagactcccgttaaatacttattcaagaataatgtgtggcaagcgataaagattacaagttacccaagcaacttgaatatgaatgagaatagaaaatcacagaagcccagcttcttccacttagcataatcatttgagtcatccatgttgtcatgtgtattcagcagcttggtccttttaattgctgagtggcaatccatagtttactcatccatttactagtggaagatttgtgttggttccagtttttagtgacagtggttaagctgctgttaacattcacctagaagtttttttgttttgttttgttttgtttttgagacagagtctcgctctgtcgccaggctggagtgcagtggtccgatcttggctcactgcaacctccgcctcccaggttcaagtgattctcctgcctcagcctccctagtagctgggattacaggcacatgccaccacgcccagctaatttttgcatttttagtagagactgggtttcaccatgttggccaggatggtcttgatctcttgacctcatgatccgcctgcctctgtcccgcaaagtgctgggattacaggcgtcagccactgcgcccggcctcacctagaagtttttatgtgaatcaaagattttgtttctcttgagtacattcctaggagtgggatagctagttatatgataaatatgtatatatacttagtaggaaattgccaaacaggtttgcaaactggtggtgccactttgcattcccaccaaaaatgtatgaaaattccaggtgattggcatcctcaccagaatttgatattgtctgcagttttgatgtttttgctatttttttgttttgacttgggtatacggtgcatttcattgtgtttttcattttcattctcccaatgcaaatgatgttgtgtattttttcatattcttctttgctacaggtatattttctttagtgaagtgtctattcaaccctttggcacatttttgaaaaactggattgtatgttttcttactgaatcttgagaattctttgtatattcttgatacaaatcctttatcaaaaatatggttttggccaggtgtggtggctcacgcttgtaatcccagcactttgggaggccaaggtgggcaaattgcttgagcccaggagttcgagacaagcctgggcagcatggtgaaaccccatctctacaaaaaaaaaaaaaagtacaaaaaatacaaaaaattagccaggcatggtggtggcacacgccttttgtcccagctacctgggcggttgtggctgtagtgagccatgattgtgctgctgcacttcagcctgggagacagagtgacaccctgtctttaaaaaaaaaaaaattatatatatatatatatatatgtcaaatatatatatatatatgtcaaatatatatatatgtcaaatatatatatgtcaaatatatatgtcaaatatatacatatatatgtcatatatacatatatatgtatatatatacatatatatgtcatatatataacatatatatgtcatatatatacatatatatgtcatatatatatacatatatatgtcatatacacatatatatgtcatatatatacatatatatgtcacatatatatacatatatatgacatatatacatatatggcatatatacatatatatggcatatatatacatatatatggcatatatatacatatatgtcatatatacatatatatgtcatatatatacacatatatgtcatatatatacatatatatgtcatatatatacatatatatgtcatatatacatatatatgtcatatatacatatatatgtcatatatatacacatatatatatgtcatatatatatacgacatatatatatgtcatatatatatacatatatatatatgtcatatatatatacatatatatatgacttgcaagtattttctattttctcccagtctggcttgttcttttgttttcttagcaatgtcttttgaagagcagacatttaaaattttgatggaagttcaatttattgggtctttttcttttttgaattgtgcttttggtgtcaaatctaagaaatgtttggtaaacccaaagttacaaagatctgtcttctagaaaagaggtgagcaaactttttctgtaaagggccaaatagaaaatattttaggctttacaggtcatagtcactgttgtagcatgtaagcagccataaaatacataaatgaataggtgttcagtaaaactgtttttacaaaaacagataaagatttggcccatgagccttagtttgctaactcttgttgtagaagatttaggtattgcagttaaggtatgatccatttctagttactttttttatgtggtgcaagcaatgggtggagagttttgtttttgtttttgtgtatgtatatacacttattctagcattatttgttaaaaagactgttctttatccactgtattatctttacaccttgtttaaaatcagttattcatgggtgtggatttatttctgaattttcttttctgcatctgtgtctgccccttctttatgactatactgtcttaattactgtggctttgtagtaagttttgaaattgggtagtgtgagtcctccagctttattttttttttcctcaaatgttttagtttttatagctcctttgcccctccatataaattttggaatcagcctattaattactaccagacttcctactgggattttgattgggattgcattgaacctatagatcaattttgacatcttaacaatattgagtcctgttaccagtgaatgtaatatatctcaccatttatttcagttttttgtttgtttgtttttgttttttttgagacagagtctctctctgtcacccaggctggagtgcagtggtgcaatcttggctcactgcaacctccacttcctgggttcaagtgattctcctgcctcagcctcctgagtagctgggattacaggcacgtgctaccacgcccggataatttttgtatttttagtcgagatggggtttcaccatgttggttgggctggtctcaaactcctgacctcgtgatctgccagcctcagcttcccaaagtgctgggattacagtcgtgagccaccacacccagcctatttcagtattttaaagtgtctttcatcagcgttttatggttttcaatatacaaatcttttacataatttgataggtttatacttgagtattcatgatttttagtgctattgtaaattgtaaataatactgttttaaagtttttaatattcagttcattgctgttatatgaaactgactgatttttctatatttccttttgtatcctgtgatcttcctaaactcacttacttgttctagtagcttttttttttttttttgtagattctctggaattttctgcatagacaattatgttgtccacagatcgaaaccatttattcctttctaatctgtacgccttttcttttctttttttctctttttattgtttgctcctccttcccttcatgtgtcactttccacctctgtcccttctctccccttccctctctcactcctcccttattacattggctaatatgaatagatctcactactaacagccgagagccttgcgtgttcttgaacttaaagggaaagcattcagtcttttgctaagaggatttattatgactggatattgaattttggcaatttttttccccatcaaatgaaatcatatgatttttctaatcagctgataggatgacttccttgattaatttttgaatgttgacccaactttttatgtatttatggattcaatttgctaaaatgtggttgaagactttttatatgtatgtctgtgaaggacatggtctgttgttttcttgcagtatctttggttttggtgtcagggtaatggagggctcattaagaaagttgagaagtgttctctcccctttcattttctgaaagagtttgtatagaatgctataatttcctctgcaatctgttgtactgctttagctgcatctctcaaattttgatttgttggttgaaaagaagccatgtaggaatgtggggtcatctgtattgggagtgcaaatgatatggtttgggtctgtgttcccacccaaatctcaccttgaattgtaataatccccacgtgtcgtgagagggacccagtaggaggtaattcattcatgggggcgagtctttcccgtgctattcttgtgatagtgaagaagtctcatgagatctgatggttttataaagtggagttctgtacacgctgtcttgcctgccaccatgtaagaggtgactttgctccttcttgccttctgccattgattgtgaggcctccccagccatttggaactgtgagtccattaagcctctttcatttgtaaattacccacgcttgggtatgtctttattagcagtacgagaacagattaatacagtaacatacatcacagtaggtatgagccaactacttgtgccttgaggtgggcagatttcctgagtgaatgtaaacagttctcgcaaatcagtctggagaatctgaagagaaccaactgaccatggccacaaggctagccccagtatttgtttccactcctagtaagctgtgctaaaatcatttcagcagaagtattttgttgacttaaggttagattccaagttttaaaagttctgaaggttggggctaacaaatgtgaggaagcttcgaatatgggaagggacctgtagaagaaaatcagccatctaagttatttaaagtaacagctgttaatttttatcagtgaggtcagtttagagataaaaacaatccaacacctgtaatcccagcactttgggaggcagaggcaggcggatcaccttaggtcagagtttgagaccagcctgggcaacatggcgaaaccccatctctactaaaaatacaaaaattagctgggcgtggtggcgggcacctgtaataccagctactcgggaggctgaggcaggagaatcacttgaacccaggaggcaaaggttgcagcgagccgagattgtgctaatgcactccagcctgggtgacagagcaagactctgtcttaaaacaaacaaacaaacaatcaaaaaatccagcaacaattgatttagatttgtagagcaggagctatagactgagaagaacaaacgagaatttgccttttttacatggcgtagaaggatcatgggagaatcaaattataacagtgagaacatgtaaacacaatctgcgaggcctttcggtgcttgctagggggcaccacagtacaataacataaagatcagcaataaggtgggaaaaataaagaaaagcagagctatcagaaaagtaatcttgttcttccgtgttgggtagacgccaactactttgtgtcaccatctcttcaatctgagggtcttgggttatagcggccttcatctatctgatcatctcctcccagagatttttaagactcaagagcttcagatctcctgaaaaggtccatttcatcaagactcaaaggggtttttattttctgatattaaatgattgtttaaaatttctcatgtgatttctcagattttattattctatgtttagacaactaaaaagacttttttcataagttttaaggtaattaaaatttggaaatgccaaaaaaaaacctaattatttcgtagcttcctccccaccccacacccccccaccctctgtataaaaaatggcttctggtatgtttgtttattttcacagaaaagtctgagaagtgtgttatgattatttaaaaaaaaaaagtcttagaaaagttgaggttagctaaatagagtagtaataattgaagactgttttcatatatgccatcacacccaaggctatcaagaggcaaatctcagggttgccttgtggccattctaggatacctgaagaacatttagttgtaaaccacacattggcaattttattattctcaagttggaaaaggcaaaataagagttaacaagcagacaaaatatgaacataagtaagaagtactaactgcagaaaatttttacaatatcatgaacacatagcaacagtaagaatgatggtgataagaaatttaattttttcaaaaaaactttatgttaaaatgattgaagagtgtgctagagatttcacacttttatttttgaaaagaaactctttggggatagaatgagattggtcattttacaggaagagtatactaatttgaatttgactgttttgttctatcatccaaataatgatttgctctgaaggatttttcttccatgaatgtaccttcctaaatatttgtttaatgtaatatttaaatatgtaataaatttgaagtcttaattcaggtttaaaatacaattacctttatgaacatattaagtaaaacaatatatattactgaatttaccatcttaatcaaattgtttctctaataaacagataagccacgtttgttatctcatgagatacatatctcatgtattattttataactatgttattatcttaactgttttgtcagttactacctcaatgaaaataactcatttcctctaaacacagagaactggaatacatacaccaaaaactagtttttttctccattagtatacattttacatcttataaatcttaagatagtaaaaataaattaatattatccaaaggcaattgcacttggtttctctttactcatcctttaaaataaaggggacaagtataaaaccaggtttcctgaccagttttgtatatttttcttacttagaaattgtcaaagtatctaaacattgtttattaactctatttaatattagtctaaacttgtaaagttaactgtggatctggaaactaaagctcatattaagtctttatggtttgcagcattaataagaatttcacatgattaaatctatttgacccataaaccagtgcaagaattttagagattttaaatcctaaaaacattaaatccaattctcatacaaactagcactttgtactaacatttttacatgatgataaagtcaataagacaatatagagctgttttttaaactaaaatcatttcaatttgtccaaagggatatgatcataaatgttacatgaactcttttaatagaattcattctaattttgatacatgatgaaactatgtattcataaaccaaaattttaaatcatgtcaaaaggttttaagtttcctgctttttgtatcttccaacctagtaaccaagaccattattcaaactaacagttaacttttttctcctatattagttttaggaagaaaaatttaaagaaccagtgccagtaaaacttgacaatttttaaaaactcgtttgaattttatctggactgtatgagtatgtaaataacatcataatacttttttattaattcacatgtttttctaagagcaggagacagagaagtagaaggcagaaaatgagagccagctatgggaaggtcagacagggcaaaagctaacctaccacctgaaaatgttacagttctgaaaaattgaatcttttaaatacaattgtgggatttatagtattacccatgtcaatttgaagagtttgtttctgtcctactattaaagatacttatcttcttgggctaagtggatcgttggaatttggatgctacagtacccgtttggtgaccccttttataatcactcagaggcttctggttctttctcttcttgactagaatggaaccattttccagtgactttttaaattcacaatatgaagtttttgtatcctagacccacaaaaagatgagctgcttgacagaggtcaagaaggttgtggagtgggtgggctgggacaggattcgggaatccttagcagtggcacaaagttctgaatacagcctgcgttagacatggacatattgggaagtatttgggtcttcttcgtatggagactgccattttaagggactttgagttagactgtatatttcagaggctggaggaggaggagggaagacgagagatacaaacagaaaagtgtggaacagtatttgggtggatcagcaagtgaggcagattttaagtatttcttattttattggcctaaatgcagtcttctagggaagggtaaaagtctggattatatttagaagcctcttcataccaattttttaagctgtattttaaaataaattttatactcatgaagaattgcaaaagccaataccaccaattcctgtgtgtcttcacccaggattccccagatgccccaatgacaagatcttaggtaaccttgatacattatcagtactaagtaatgggcattgattaatgctgttaactacactgcagactttattcgaattttgttacatgtctttttccagtgtatatttatatgaaatttagttatgttatagatgcatggtaactactgccataatcagtatacagaatatcctatcaccacaaagtctcttatgctacagttttataatcataccttcacagatgtgacccccagcaaccactgatgtgttctccgtcactatcatttggccatttcaatagtgttaggtaaatggcatcatgcagtacatagccttttaaaattggcttttttttttttgtactcagcaagtatccttgagagtccttcagcttgtcacatgcatcagtagtttcttcttttttattgctgagtagtattccattgtgtggacgaaccactgcttatccattcacttattgtaggacattgtggttatttctagctttcagctattataaataaagttgctataaccatttgtgtagatttttgtatggaaatgtgtttttatttctgtaggataaatacccagcagacttatggcaagtctgtgtttaactttataagaaactgccaaaatctttttcagagtagcgttttatattcccaccatcaggatatgagagttctagttgttctacatcctcactaacagcattgtggagttttgggcattatttatacatattattctaataggtgtatagtggtatatcaccatgactttaatttgcattttttagtagctacaatgttaaacatctgtgtttaacattgaataccacatgccatttgaatatcctctttagtaaagtgtctggttttgtacattttctaattggattttttgttttcttactgttgtatttagagttatttatatattctggatataaggtccttgtccgaactgcgaattggaaatattttctcccagtctataccttgtattttcatcctcttaacgtggcccttctcagagcaaaaggatttggttttgataaagtctactttattgatgtttttcttttatggtcatgattttagcatcatgtctaagaactccacctaatcctagcctattaaaagatttatggtttcactcttcacatttggatctgtgatccatcttgagttaatgttcgtattaggtgtgaggtttaggtttaggtttgtttttatgaccaagaatgtgcagttactctaacaccatttgttgaaaaaatcttttctcctttgaattggatttgcccttttgttaaaatcagttggccacagttgcatgggtctatttctggacacactattctgttcctttgatctgcatatctgtccctccaccaggacttcattgccttaactgctatcactgtatagtattagaagactgaatattgtgattccaccagctttactttcttttgtgttttttatgctttcatgtaaatttgaaaattttctcatatctacaaaaactccttctgaggttttgatgaaagtggtattaagcctctggatcaacttgaggggaattgacatcttcaatatgttgaatcatctttcaatccataagcacagtttgtttctgtattgacttaggtctttttaagattttcttcatgagagttgtgtaattttctccgtacaaatcctatgcatagtttgttcaatttagattgaagttatttttttggagcagctgtataaagggttttgtgtttctaatttcagtttcaatacatttgttgctagtgtatagaaataagttccatttgtgtgtatgtgtgtgtgtgaactttgtatcctgcaaccctgctaactcacttattagttgttcccttccctgtttcccagaagagatggtataaagttactgttacttattcttttaatgtttgttacagttcagcagtgataccatctgggcctggagatttctttatcaaaaggttttaaactgttaattcaatttttttagtagttatgagactattcaagttatctgttatcttgggtgtttttatagtttggaattggtccatatcatctaaattgtcaaatttatgtgcatagagttgtttgttgtattcctttattatcctgtaatgtctgctgggtctgtaatagtattccttctttcattaccaatattgatcatttatgtctttcttgcactttcccccttcttaacttcctgtctcccctcctctcctctcctctctccttccctcttctcttcttctccccaccttttgtcagccttgcttgaggttgatcaatgttattgatcttttcaaagaataaacttttgctttcatttttctctattgtttttctgttttcaatttcattgactttttactcttacgtttattccttcctcctacttgctttgggtttattttgctcttccgcttgtagtttcttgaggtggaagctttgttttggatctgagaccatcttccaatataagcatttagtgttaaatttataaatttctctaaaagtgaaactgctttaactgcatcccaaagatttttatatgttgtattttcattcccttcagtgtatttttacatttcctttgagactttctctttgaccattggagtatttaaaagtgggttgtttaatttctaattgttttgtgattttcctggtatctttctgttattattgatttctagttagattcattatgaacagataatatattttttatgatttcagtccttttaaatttgtgaaggtttgttttacatgacccaggataggatgtggtccatcttggtatctatttattagttgccaaggccattttttccttgctgaggggcttggcttagagataacttacatctcttttgcccacattccaatagttagagctaaatcatatggccacacctaactgcacagaaggctgggaaatagattttagctgtgtgacttagaaggaaatgggaatcaagtctggaaattacataacactctcttgtttttgacttttttaaaattttctccctttattcccattcccaatcctatagttatttaactacataccctagtgtatttaatatatgtccttctgatctaccttctgtacattgaattagatttttatgaatctttcaaaaatacctaagatttcttttagtttttttttacatgtttataaatggttattgtctgatatagtttggatgttgtatcctctaaacctcatgttgaattgtaattccctttgttggaggtagagcctaatgggaggtgactggatcatgggagcagagttctcttggatggtttagcattatccccttggcgctgtctttgcggtacttctagcaagatctggctgtttaaaggtgtgaggcatctttcccctcgccctctgttgctcctgttttcaccatgtgacatgcctgctcctgctttgcctgctgccatgagtaaaagttccctgacgcctctccagaagctaagcagatgccagcaccatgcttcctgtacaacctgcagaactgtgagccaattaaacctcttctcaggttttttttttttttctcagtctcaggtatttttttatagcagtgcaaaatcggcctaatatattgtctgtatagcattctgatgattagttcttcactgtgtattaagattgacttatgttgctgcatgtatggctaattcatccctctgactgcttcagaaatattccactttattcatatttattcaacattttacttatctattcccccgtgtactaaaagtcaaaagcattactaatttaataatgtcatatttgttcttctaattattttttgctgcataacaaagcactcctaacttagtggcttacaatgataacagtcatttattttattcaaaaatctgcaatttgggctacaattggcagaacagctcatctttgctccttacagtattggctggagttggcgatgactcagcagatcaaggctggaagactcattctcgtcaggtggttgacgttggctgtcagttggaacctcacctggggctgtttgccagaacacctacacatggcatgtccatggagctatttgacttcctcactgcaatggctgggttccaagagcagttatcccaagagaacaaggcagaagtgcatgacttttagaatcttgtctcacaagtcacacagagtcacttccaccatcccctgttggtcaaggaggacatgaaggtcttcctaggagcacggggagaagacagacttctaccacctgttggaaggagtgtcagtgttacatagcaagagaagcacatggaatgggatgtattgtgatgcccatctttggaacagacaatttggcatatctgtgaaaattaaaagtctgatttggatataacaaatgagactgattagcaaagatgtctaattataaaagttaaagttaaattccaaatatactttttttttttgccaaagaccaaagactaggttatagccaaggaattctggctccctatatccctaggctttttttatttttatttttatttcttttctgagatggagtctcgctgtgtcacccaggtgcagtggcactgtctcggctcactgcaaccttcacctcctaggttcatgcgattctcctgtctcagcctcctgagtagctgggattacaagcgcacaccaccacacctggctaattttttgtatttttagtagagatggggtttcactatgttgcccagactggtcttgaacttctgacctcatgatctgcctgccttggcctcccaaagtgctgggattacaggcgtgagacaccgtacccagcccctaggctttatttctaaggttatcaataccttcttccccttgatccttattttttatgtgatcaacataatacatgagatcaaccagttcaagcaacacgattacaaaacctatgcacaccttgagtgtaataaagagaaacagagcttttttctctttagaaaatatatcacatgagaaaacaatagtaactagttctcaacctctttatccttttcttttgcgccgctggccagaagatctctgatgtattgggcattctgactagataccttcagtatacacatgactattctgaaagcttattatctacatcactagaaaatagatccataacttttgcagtccttgtgcctttttctctgaacttgtctctcctgagactctattaaatacaatggtgcttgaggaaaacactgccagaatagctctgcctctcataaggtgatgtaagaaaaatgggaaaagtgttctagcaaagtggtcatgaaaagattgtagtgagttcaaccctgtcaagtcctgatttatgtatatcattttacccatttctcttaaagtccagaaagttcatacccaaatggatgaaaatgctgatcactcttccttttaactacataaagctttcaccaaggcagaaaatcacagctaaaaaattaacataagatggaccaacccgatttgtttcttttcttgaattgctaagccaagacgagatgagtgttgggcctttgtttttattttttaatgggtaatttcaaatatatatgaaagtagagaggatggttcagtcattcctcagtatctgtgagggattggtctgaggaccccctgtgaataccaaagcctgccgacacttaagtccctgctataaaatggcacagtatttgcatataacccatgcacatcctcccacatactttaaatcatctctagattacttgtagtacctaatacaatgtaaatgctatgtaaatagttgtcatactgtgttgtttaaggaataatgacaagaaaaaaaaatctgtacatgctcagtacagacacaaccatcatagacctaactacacttttgatctgatttgaatccgcagatgcagaaccctacagagggctatgtagtgaaatgccccacccatcccctaacttcagcaatgaccaacccatggccaatcttgtcatctgcactcatccccaaatttaatatatttggggatgtcatctgcatccccaaatatattaaaatgtatttaaagcaaaaccttgatatcatttcatccataactgcttcaatgtgtatttcaagaaagggactctttaaaaacataactacaataccattatcgcacattttaaaattagcagttgtggaggcaaaaggtgtgtctataactggggagaatgaagcaaagaatataagtggacatagaacacgaaaaacaaaacaaaagactgagtctctgaagtcctgaaaggggttccagtccacaggtctccctttgatgttgagagtcacctcatctctttgtatcattctaatcccttctttttgtattacactgagttggacttcagtaattttttttgagacagtcttgctctgtcgcccaggctggagtgcagtggcacgatctcggctcactgcaagctctgcctcctgggttcatgccattctcctgcctcagcctcccaagtagctgggactataggtgcccgccaccaagcctggctaattggacttcagtaattttaatttggaagcatcatggctaatactactgctatctggacagcttagtgttactcttccctccatattttttgatttgtttatcataccattcaatttagttaatgccagtagctaccgtttatcgaacaccgatcatgtacccggcactctaccacgtgtagagattaaactacaccaatttcacaacaacccaatgaaaaaagtatttccctatcttgtagataaagaaatgaaggtctaactggattaggtaatattcccaggttaaacacttaacaagtgtggaaccaaaatctcacgcagatctttctggccctcagcctgttcccactatgccatgctgcatctgacaaacgtgatttttattttctaaaaaagccagtacaattctgtggtgcctgctgttcatcatattgtttcctcatcttcctaggcgcgcaactactccttcatggcacttagccttggtcccgtgattgggttctagcctatggaatatgagtaggagacatgtcatgtttcttggctgggcccaataaaacttcccatttgctgtcctttttgctctctctgcttttgccgtctggctgatgatgcccaaggtgaccttgggaaccacacattggcgatgacagacggacatcctgagtctctgagtgactgtatgggacagagccgcctgccaccaatctgaaacctctttggactgttcacatgagcaagagataaacttctattgtatttaagccgttgtatatttagcattctatttattattacagcagttagccttccttaacttttatgtactgtttcaattatcttttactgggtaacaaactacccccaacatttagcgacttaaaaccacagtcttaaattaactcatgattctgtgagtcagaaatttgggctcttgcccctacttgttcatgcggctacagtgagcaggtgggaagctgggggctagttggtccagcattgcctcactcacatgtctggaaataggtgctggctggactgtgtgtctccagcaggcctgcctgagatttttcacatgttggtggaagtgttcccagcagcaagagagggcaagccccaatctgcaagtatgtctcaagctcctgcttgcatcgtgtttgccgatgtgccattatttagtgagtcacctgcgtagaggggagtatgatttattggggactatttactgcaaaaatctgccatatgtacaagagttaggcaaatcatttacgtatatacaacatattagtcacatatttaataccagagtcccactctgagactgttttcataatccctgaatcaagttctatttaataatgtaatcgtttattttttacttttcaggtctatatatgagaataaaataattaccaatacatcatacatttaggttagtagaattgggaattaaagaaatgaacccagtttcatctttgccattaacattgagctccctttgttttaagtcagatgcagttgcagtaaccctccctagactccataaccctcttgagctactgtcatatcctgtcccttccatatcatatttctttctttcctttttttttttttttttttttgagatggagtcttgctctgtcacccaggctggagtgcaatagcgtgatcttggtcactgcaacctctgcctcccaggttcaagcgattctcctgcctcagcctcctgagtagctgggattacaggcatccacaaccacacctggccaatttttatatttttagtagagacggggtttcactgtgttggtcttgctggtgtcaagctcctgacctcaggtgatccacgggcctcagccccgcaaagtgccaggattacagacataagccactgtgccccagcccacattgtatttcttgaaggatatcctcatcttccagttatttctcaatccttttcagtctggcttccttgcattaatttatgaaaacagctcttatgagggtgacccatgaccttcctgtcactaaatccatgagcatgaaactttcttactggtggttgacttcttctcaacaccacgccaccatccgtctgcttttccttcaacctttctggccttgtcttagtttcctttgaagactcatcctcctttaccttgtctttaagtgacagaattcctcaagacttcgtgcttggtcttttctcactctactcttgccctaggttggcctcatctttgaccatgtcttcaatactatctatatcctgttgagtcccaaacttgtttctctacctcaggcctctgttatgccccagccctgtgtatctccagttgtttatctcacatgcacctcaaactcaatttatgtccacaccggccctgtgcttcctcctcattcacccttccctccaaacaaaatccagttcttttctagtagtttcaaaatgaccccacaatccaaaagcccgtgaatcattcttgactccctgtaccatcagtgccccctgtgccatccttcacccattccggtggtgcccctttctattctgggaaaatctcccactgtgagtcatctgggtgggtgactgccccagctcccactatgaaagacaaaggggctaacatcctccctctctctgtctcctgtcagctaaggcattgcctgtgacctgaccttggttgattggctgtgtctgccaagaactttgaatattaagagtaaaaccaagacggtactattagaagtcattcatgaatgtatcagtggagtactggtggtgttgatcctgcttcctgtatttcttagccttccagaactatcttggttcctgccccccccaccacattttccatgctggtgtgctagcccccagtggatttatcctataaatgcccccatttgcttaagatacccagagaaggatgttgttatttgccaccagagaagctgtgaccaatataggcactgatgttccaggtagattcagatgccatcacaaaattttctccactaatcggcgacctagccacagaatgcacaaaacagctctcaagatctctgcatgcaatatcagtgcctatgggtccctgtggctatttcttagtgctgcccatgagccaccatcatctctgcctcaacttctgcgagagcctcccagctggttgtctcataaggtcctcaacttcatactgcagccaaagccatcttttcggaatgtaaacctgttggtgccactcctctctgatgaaatctttcaggggcttcagatttctgtgaaagaccaaaattcttaaacttttttgacctttactcgcaataaagacagtttatattgaaactcagtacacttgatgtatattttataaaagtgtcacaagacaatacttaccctttatttgattaacaaatacttgcatggcagttactgattgccagacactgttcttgctgtgctgtgaatagtaactcatctagtccctgtaagaacccatgcgatgcacatgttcttatgaccactttaaagctgaagccacttgggcacagagaggttcagttgcttgtctgaggttgtacagttaatgagtggcagtctgggtccagacccgtgcttttcactgcttcttggtgctactctgtggtggactctgatattctattctgtttcattttttaaaaacagaggttgggcgtacaccccattgctttcacaacctcctagcaggtcacagtatacagtttaagaaacactggcctgtcttggggtgggcccttaccttgtatttcccagctccacctgtgctgtgctcctccttgagttctatgatccagttttcatggccaggctcctcggagccatggggccttggcccatgctgttcatttactgaacctcactgtcctccctgcagattgtgtccttgatttcaagggagaacggtacgcctatgataggccctgaaagcccttgcacctttccttgggcactcatcatggttgcagtttggcagttacccacttggtggtttgaataaatatatccatcttccccactagaccgtatgtgctgcgagtgtaggctctctgcgtgtttttagttcactgttgagagaggtgatctgccctgagtcctgagcatccagcacattcttgctgggtaagcacacaaggcgaggccctgaccactctactcagactgcttctcagcagacattatgttcgcagcagacaaccttgaggataaagtagtagctccctccagttcggagagcagacttgctactgcttgctataaaatggcaggctcctcaagctcagtgttcttcagctgccatgcaaagccactgtatgtgtagcatccacctgtgccccagcacactgtcttcgtgggctacggggcacaggggctgatgcagacatgctcgtgctcatgttgtctactgttctgtcgatcataaagtcctgtgcttctgacccaggaggcttgcgtcctgtgccagcatccacaaaacagtaacagtctaacttactaacttgcaagtagggtaaaaagtccagacatgacactaaccatttcctatcagccagcacagtacctggcctaaagcagatgttcagtatttatttgtgaaacaaatggatggttggatagatagaaagtagatggactttgagctctgcttaaggtgttgcagggatcaaaccctttcagaaatgccaaccataccctggaatgattctggaactgtggggaagacaattgaggagaaatgaagattatcttgacctgaagtttctcatcctttgtgtttctaagtagagtttttggatcagccagctcctcttctgatattaaactatctgacatgggtagtcctgtgagacagttgcgttttattctgaattgcctgaatgagtctgatgtccccaacacctagcgaagctcagcaatgatgcatggggtgggctgatgtgggaagtaggcagcccggagtggcagaggcctgggttctattttaaaagggaattctgggtatcactgggtctcttagagggggagttagggagggggaactggaaagtgagaaagggaagtttgcacagcagcaaagccaaagttagcctcaagttctccccaaaggccaggtggcagtaattcgggacatgttgcagaaattcatctctgaagcaactgggaccagaaggcttcaggcaacaactgtccctgctaggagggcctgttcccctttcacaaacagcactgcttttcctaatataagcacagttcttgcacatagataaaactgtctttaggatagagtgaaaagagaataagaatgttaaatttataactattaagatgtattgaaagttgaataatgtgcagaaagcaaatcgctctacacagaaagagagggtgatagaagggttcttggagacaacaagcatgagaagacttatctagggtttgatgagattttcagtaagagctttatcatccccacaggggcaaaattaattcttgggagacaaaaaggaacttagttattcgatggtttgtggccttcagagggttgcagttgtacataaacagaaacacagtatatcagtggtattaaaatttcaagatgggagagagattggggaaaatatctaaaatgtctccttacaggggtgataatgagaagcaatggactagaggattcaagtggaagagtcttcatcacccgtgagttctcctgggaaagtgaattggaaacaagctaagtgcattttctcagcagaaggaagatcttacacatcacctagtgaatatccagttgggaatctgaaagcagcagtgggcaggtgagctgtccccacagctgttcagcaggtgatgagcttactggctccaggaaacgtcagtgtgcttaggatgcagctgatggccttaggcaccaggccagtcctccctggacacatcaataggatggtggcaatagagggtggaagttaagattccaggaattagcccacccacgtccttccttgctgttctattctgttctcatctccttccttcctactctcctctccggctcactcattcatttttgctgcaggcaggggaatcaggagcaatgttcatcagtgaatgaaaggagtgctggaaagagaaccaaatgctgacatcaaagaaccttcaatcatatgattttagtgtggaaagccagatgatacactctaacatctgtgaaatcaaatcaaaatgcaaattggtttttggctttcctgatctatgtttctggatatgtgttcactgggacatgtggcttcttgacagaataagactgaagacaatttcaagtatagacaatagttagttccaatcccacaggtattcagccttggaaatgggttccagtctctgggcttaaaagactggactttgctatcagtttgaaaatggcagtgtcgtagaggggctcttaatgttaggaaggatggcctggcagagtctcttttcttctcttgccctatccctgatgctcattaaggcagtgctagaggaaaaagttctcattaggtaacaggagttgctactgggtaccgtttaaagtgctttacacctattaattcatttactcacaaccagacaattctgcaaggggagcactgttttataaaactcttatggatggggagctcaggtacagaaatgctaagtcatttgtccaagctcacacagcttctggtagtaccgggatttgaacccagatcctcagtctccagagaacttgccacaatagtgttcttttaaggtgccctcagccaactcttgcacttattgatatctttgaactcatggatgaatgggcttaaaattgtttttaacacaatgtactttggtttgagccttatttaaatagaacaatgtgccagtcaccagaccccatgcacggttgaatcctttggccacttacataatggattaattactggacacatgctgccctcttgtgatgaaagtcactcaattgcaaccttctccacttctctggaatgtacccagttacttcctttgttccagtctcatggttgggggatggaggatctgtagccagtgacgaagctgttggctctgtagagccaagatgtcacctgccattctgtgtggtgctcccgggctgttggctctgtagagtcaagatgtcacttgccattctgcagggtgctcccagcctgtgactccagccatgagacatgaccccagagtcctgcaacttgtagcaactgaatataggagaaaagaccaggcttcctgaagaaatcctaggaacattaaagagaccccatctgtgtccttggggaaagagctctattttgcataacttgccctagcagattggaaccagtgatagtgacagaaggcagccaaatgcctaggtaaatgggacaggtcccaggtgaaaccccaccttaaagccaaaaaacagcctgaggggtgaaaggccggattgctggtccctgatgaaacccgcgacctagagtgagaacttctgtttctgtttgcccgccctttcctgattgattctttctgaataatgccttttaaccaatcgaatgttgccttttccaatactacctatgacctgcccctttcccattctcagcccataaaagccccagactcagccacagtggggggactttcccaccttcaggtagggggaccacccctgtatcccctctccgctgaaagctgtttcatcactcagactccttgccttgctcactcttcgattgtcaacatatcctcattcttcttgggtgcgggacaagcacccgggaaccagtgcacaagccagacttgtcccgggcaggctgtctcctgcagcagggtagcatggctgagcaaggcccaggtggggcgtcgctggccagaggtccctggcttacaaagtggccaagaaaaaaatcctgtgtcaccaggtgatagacttggcagaaagatggtcagttgcagagtgaactgaaggtcacaaaccctatcagaacagagggaaaataaaaccagacagaagccctgttcttgttctaccagtagttaaagtgaccttgagtgagtatcttttgctctctgacaccagtttcctcactgggagaatgaggagattgaaatcttagatactgccaggaaaaacattctgcatttgcctagatcccttccctgaatagatttgatagttttgttttgctttattttattttattttattttttatactttaagttttagggtacatgtgcacattgtgcaggttagttacatatgtatacatgtgccatgctggtgcgctgcacccactaactcgtcatctagcattaggtatatctcccaatgctatccctcccccctgcccccaccccaccacagtccccagagtgtgatattccccttcctgtgtccatgtgatctcattgttcagttcccacctatgagtgagaatatgcggtgtttggttttttgttcttgcgatagtttactgagaatgatgatttccagtttcatccatgtccctacaaaggagatgaactcatcattttttatggctgcatagtattccatggtgtatatgtgccacattttcttaatccagtctatcattgttggacatttgggttggtgccaagtctttgctattgtgaataatgccgcagtaaacatacgtgtgcatgtgtcttcatagcagcatgatttatagtcctttgggtatatagccagtaatgggatggctgggtcaaatggtatttctagttctagatccctgaggaatcgccacactgacttccacaatggttgaactagtttacagtcccaccaacagtgtaaaagtgttcctatttctccacatcctctccagcacctgttgtttcctgactttttcatgattgccattctaactggtgtgagatggtatctcattgtggttttgatttgcatttctctgatggccagtgatgatgagcattttttcacgtgttttttggctgcataaatgtcttcttttgagaagtgtctgttcatgtccttcacccactttttgatggggttgtttgtttttttcttgtaaatttgttggagttcattgtagattctggatattagccctttgtcagatgagtaggttgcgaaaattttctaccattttgtaggttgcctgttcactcagatggtagtttcttttgctgtgcagaagctctttagtttaattagatcccatttgtcaattttgtcttttgttgccattgcttttggtgttttggacatgaagtccttgcccatgcctatgtcctgaatggtaatgcctaggttttcttctagggtttttatggttttaggtctaacgtttaagtctttaatccatcttgaattgatttttgtataaggtgtaaggaagggatccagtttcagctttctacgtatggctagccagttttcccagcaccatttattaaatagggaatcctttccccattgcttgtttttctcaggtttgtcaaagatcagatagttgtagatatgtggcgttatttctgagggctctgttctgttccattgatctatatctctgttttggtaccagtaccatgctgttttggttactgtagccttgtagtatagtttgaagtcaggtagtgtgatgcctccagctttgttcttttggcttaggatcgccttggcgatgcaggctcttttttggttccatatgaactttaaagtagttttttccaattctgtgaagaaagtcattggtagcttgatggggatggcattgaatctgtaaattaccttgggcagtatggccattttcacgatattgattcttcctacccgtgagcatggaatgttcttccatttgtttgtatccttttatttccttgagcagtggtttgtagttctccttgaagaggtccttcacatcccttgtaagttagattcctaggtattttattctctttgaagcaattgtgaatgggagttcactcatgatttggctctctgtttgtctgttgttggtgtataagaatgcttgtgatttttgtacattgattttgtatcctgagactttgctgaagttgcttatcagcttaaggagattttgggctgagacaacggggttttctagatatacaatcatgtcgtctgcaaacagggacaatttgacttcctcttttcctaattgaataccctttatttccttctcctgcctaattgccctggccaaaacttccaacactatgttgaataggagtggtgagagagggcatccctgtcttgtgccagttttcaaagggaatgcttccagtttttgcccattcagtatgatattggctgtgggtttgtcatagatagctcttattattttgaaatacgtcccatcaatacctaatttattgagagtttttagcatgaagggttgttgaattttgtcaaaggctttttctgcatctattgagataatcatgtggtttttgtctttggctctgtttatatgctggattacatttattgatttgtgtatattgaaccagccttgcatcccagggatgaagcccacttgatcatggtggataagctttttgatgtgctgctggattcgttttgccagtattttattgaggatttttgcatcaatgttcatcaaggacattggtctaaaattctcttttttggttgtgtctctgcccggctttggtatcagaatgatgctggcctcataaaatgagttagggaggattccctctttttctattgattggaatagtttcagaaggaatggtaccagttcctccttgtacctctggtagaattcggctgtgaatccatctggtcctggactctttttggttggtaaactattgattattgccacaatttcagctcctgttattggtctcttcagagattcaacttcttcctggtttagtcttgggagagtgtatgtgtccaggaatttatccatttcttctagattttctagtttatttgcgtagaggtgtttgtactattctctgatggtagtttgtatttctgtgggatcggtggtaatatcccctttatcattttttattgtgtctatttgattcttctctctttttttctttattagtcttgctagtgatctatcagttttgttgatcctttcaaaaaaccagctcctggattcattaattttttgaagggttttttgtgtctctatttccttcagttctgctctgattttagttatttcttgccttctgctagcttttgaatgtgtttgctcttgcttttctagttcttttaattgtgatgttagggtgtcaattttgcatctttcctgctttctcttgtgggcatttagtgctataaatttccctctacacactgctttgaatgcatcccagagattctggtatgttgtgtctttgttctcgttggtttcaaagaacatctttatttctgccttcatttcgttgtgtatccagtagtcattcaggagcaggttgttcagtttccatgtagttgagcggttttgagtgagattcttaatcctgagttctagtttgattgcactgtggtctgagagatagtttgttataatctctgttcttttacatttgctgaggagagctttacttccaagtatgtggtcaattttggaataggtgtggtgtggtgtgctgaaaaaaatgtatattctgttgatttggggtggagagttctgtagatgtctattaggtctgcttggtgcagagctgagttcaattcctgggtatcctttttgactttctgtctcgttgatctgtctaatgttgatagtggggtgttaaagtctcccattattaatgtgtgggagtctgagtctctgtgtaggtcactcaggacttgctttatgaatctgggtgctcctgtattgggtgcatatatatttaggatagttagctcttcttgttgaattgatccctttaccattatgtaatggccttctttgtctcttttgatctttgttggtttaaagtctgttttatcagagactaggattgcaaaccctgcctttttttgttttccatttgcttggtagatcttcctccatccttttattttgagcctatgtgtgtctctgtatgtgagatgggtttcctgactacagcacactgatgggtcttgactctttatccaatttgccagtctgtgtcttttaattggagcatttagtccatttacatttaaagttaatattgttatgtgtgaatttgatcctgtcattatgatgttagctggtgattttgctcattagttgatgcagtttcttcctagtctcgatggtctttacattttggcatgattttgcagcagctggtaccggttgttcctttccatgtttagcgcttccttcaggagctcttttagggcaggcctggtggtgatgaaatctctcagcatttgcttgtctgtaaagtattttatttatccttcacttatgaagcttagcttggctggatatgaaattctgggttgaaaattcttttctttaagaatgttgaatattggcccccactctcttctggcttgtggggtttctgctgagagatccgctgttagtccgatgggcttccctttgtgggtagcctgacctttctctctggctgcccttaacattttttccttcatttcaactttggtgaatctgacaattatgtgtcttggagttgctcttctcgaggagtatctttgtggcattctctgtatttcctgaatctgaacgttggcctgccttgctagattggggaagttctcctggataatatcctgcagagtgttttccagcttggttccattctccccatcactttcaggtacaccaatcagacgtagatttggtcttttcacatagtcccatatttcttggaggctttgctcgtttctttttattcttttttctctaaacttcccttctcgcttcatttcattcatttcatcttccattgctgataccctttcttccagttgatcgcatcggctcctgaggcttctgcattcttcacgtagttctcgagccttggttttcagctctatcagctcctttaagcacttctctgtattggttattctagttatacattcttctaagttgttttcaaagttttcaacttctttgcctttggtttgaatgtcctcccgtagctcagagtaatttgatcatctaaagccttcttctctcagcttgtcaaagtcattctccatccagctttgttctgttgctggtgaggaactgcgttcctttggaggaggagaggcgctctgcgttttagagtttccagtttttctgttctgttttttcccacctttgtggttttatctactttgggtctttgatgatgatgatgtacagatgggtttttggtgtggatgtcctttctgtttgttagttttccttctaacagacaggaccctcacctgcaggtctgttggagtaccctgccgtgtgaggtgtcagtgtgcccctgctggggggtgcctcccagttaggctgctcgggggtcaggggtcagggacccacttgaggaggcagtctgcccgttctcagatctccagctgcgtgctgggagaaccactgctctcttcaaagctgtcagacagggacatttaagtctgcagaggttactgctgtctttttgtttgtctgtgccctgcccccagaggtggagcctacagaggcaggcaggcctccttgacctgtggtgggctccacccagttcgagcttcccagctgctttgtttacctaatcaagcctgggcaatggcgggcgcccctcccccagcctcgctgccgccttgcagtttgatctcagactgctgtgctagcaatcagcgagactccgtgggcgtaggaccctccgagccaggtgcaggatataatctcgtggtgcgccgttttttaagccggtccaaaaagcgcaatatttgggtgggagtgacccgattttccaggtgcgtccgtcacccctttctttgactcggaaagggaactccctgaccccttgcgcttcccaagtgaggcaatgccttgccctgctttggctggcgcacggtgcgcgcacccactgacctgcgcccactgtctggccctccctagtgagatgaacccggtacctcagatggaaatgcagaaatcacccgtcttctgcatcgctcacgctgggagctgtagaccggagctgttcctattcggccatcttggctcctccgcttttttttaatttttatacaaatcctgattagtaagatacatattcatattattagtcctcagtttcctcattcaggaaataattctacttcacagggtcattgggaggaagtttttatttggcctgatgttcatgggtttagccaggctgggtttgtttaggcaaaggaaggacacatacacatatttataaataaaaaggttttacatgaaggaaaactgattttaaagattgtgacacttgcccactagtaagtggttatcttcaacagtgccactttaaaacagatgctgatgtgttcatttattcttccctgcagccagtcattggcatagcacacgtatgtgtatgggcggtgcctgaacactggtctggtgtgaaggttagggcagggagttttattctattcaaaagagacactgctggcttcacattacattggtctctaattttcacttgcttccaaacttactacacccttgacataggttaaaaaataatatttcatagcaacggtctgggtttttttgtttgtttttggttttgtttttgttttgagatagggtctcactgtgtcacccaggctcacttacaggctagacctcctgggctcaagcggtcctcccacctcagcctctggagtagctgggaccacagttatacatcactatacctggctaattaatagcaagaatctttgccttgataatctcattccaaagaaataactataaagaattttttaattccatagaaatattgtctatagtgaccaccaccacagtcaatctggaaacttgaaggtctaacctgaacgtctggaatattattaagcaaattctcaaactggtagaatatattaataaataatagagactaacagtgtgggaaaatgacaaaaagaactgctaatgtactctgtgattataattatgtaaaaagatgtctcaaagtaggcagagcatggaagattacatacaaaaattaaaatagctgtgttagtgttgtgttactataaacattaaatttttccctttatgacattactgacaaggctaatcccccctaaaatggagtattttcctatttggtgccacaaagccaatacacaaaattgaaagtgtgcatctagcagtgcaggctttatttgatgcccatagaactgagatgtgggagcctggctcacaaatcaacttctcagcccatgagggatagggcatccccctatgacagccaagaaatcacaaatataggacattccccatgagagccaggaagtcacagatatggagcatctttaatgaaggggttggggaattaaaagcaaggggaaaagtattcatgtcttttctgggaattaggtagataacttctcagaaccagagtttttgtcccccttttttgtccttttatggtttcttctggtcgttgtcatggtgattgtcagctgtcttggcactggggggagggtcatttagcatggaaattagattataatgaagttagaggttcttcagaggtcaagtgagctgccatcttggttcccaccactcttagctggtttggtgtcatgatggggagcttttgaccacagacatcccgtttcctaaagataaccagagttaaggtggggaagaaatttacttaggtcacacagacgttacactgggtaacaacattactttgacaacaaaaataaatgcactttataacagaaaaaagtccaggaaaaaattgagacactttaatatttgttattggtctgaaaaaacacccaagtagtcatgggaaagattagaaacttgtttaacatctttgcacttactttataatttagtatttaaatattttcattatagatccagaggggcctataaccttttcagtacttaatgtctctaagagtcttaagctggtcttgggtgcagttcattccattttaaggttgagagagtagtgttgggatgtcctctggtaaaatgagtctcagcctcctcataatgccattctactttcattaaaattcttcttcatgatcctgggttaatggtcatggttttgcctttcagctctagagcaaggtttttttcttgtaagtaagcagttggttactggaaacagacctgtggtccacctggaagtgaaccaaaatatggatgagcagaaagcgctttcccgcttggcaaaggctcatgggcctggctccatagggtggtgccagctcctgccctgttcttttgtgctgtgaagacacaggtgccccctatgttcagtgacttgacacttaaatggcagtatggtctttggtaataataatgaaaactaacagctatgcttttcactgacaaaggtaggatattgcaggaagcttgatgttgaatactttggggttttactttgagaactcctagtcctgtattggaccttgttggaaagttccctgagcctatgagtgagaaggggacaacttgggagtcccacggtgagcggaaatgagacccgctggcttgatagtaactaactgccatacgttgagcaggactctgtgccaggctttgtgccagctactttccatgactcatttaatcctctcaacaaccttttgagtcagattctctcagtttttccaaggaggaaatcaagattctggccgtcttaagagtgctccaaagccacacagctagggcgctccagagagctaataccacatggccacacagtggctccacatggctttttaaagaagttccatgtcagcatggacatgacacaagtgcaggtgtgaaatatgggagctgtagcttctttcccaaaatcaatcaagatgtcctgtggaaagaaagagatatagccctcgtttacagaattttttttttcaattgtctttttcttgattaggaaaagtctccatgtttctgctcagaagctcacctgtttctaataagagaacctttccatcaaattaggagcctggtctcagttttacaggaggaatagaaaatctaccacttgccatctggaagatcaaagtatttccaaatagaatttaagtcaataccacacaaattctaattccctagaagcagcagttttaattttgatagtctctaaaagcaataacatttggtagtttctgagtaaattttagactctcaagggtttagttttagcagtttaataaaatgtgaattttgtctgctgagaagtgaactttgtggtaagacttggcgagtctgaggtctgtggtactccccctttggccttcagtgggccatgctcattcccttctttcttctttctgggcatatttgctgcccagattctgcagtaaggagagagtggctgccttggggtgattattaggtgaatactcaggtctggggttgggagggacctcgtaggaaccaccaaaatcgagcctttaaggaactgccgccttgtagttgcttaactgtacaggatttcccccgcaacggtccccttcctaacaggctgctctacaaagccaggcttggactcataagaactcagaggtggaccgttggaggccctgcatgccttcctctgtcacgtgctgcccctggcagctcggtgcttcttgccatgggcctacctggatccgccactcagcagtgaacagccgaggaaggtagtggacgtcgaacctggcccgtgggctctaccatggcatggtgatgaaggcatgaactttggcccacttggcctgtcctggcaccagccaccccctactcacaagttatttcctctgagaagccacctgaagagattcagaggccagatggagagacctcagaggctgccacagccgcttcctgtcccttggtaggcagagaggcctgtgaaggatcaaactggtgacacccctggccttcatcaggctctcctcatagcacatcaccccagagccccttccccactgtggagtgaatcaggatggggctcaggagtcaatgagtgcttaccaagccaagtcccttcaaaaactccagactcaacccgtaagagtgttttcagtattttattaacaaatgagctggcaagaggacaagtgatctagtagtatcacccccaccctcatggagcagccaccacaagcccaccatggtggggggtgtccaacatgctctgctggcccagttcccagccgatcccctgagtcttggcgcccgtttagtcacccttcagctgcttgggaggcaggaagagacttcccctcttcacgaggtaagggagacaaaagcagccatttggatgccagggccacaggggcaagccatgccctatttctttggagggacagaatcacttcttcccaaggccagacactgtagcccatggtactcagccttctagaggagggtagcctagcagaggagaagccctgagtggaagcagcattttgaaggcatcgtcattcttagaccagctaagagctgagggcattctctatctttgccagcagacagtgagactccaggattaaaattaaaagcccgtggtgcatcctttccttgacattaactttccacaaaaccttggaggagtcaaatcccacactgcacatactccggtgtctccagctgtgaagtgggaccagtatcaatacatgggaaagtgctgagaaggaagaaaatatttcaggtataatactaatccatcaaacacgttttattcaagaagcttggcaccaactttgtgccacatcctcaactcttcaatgaaaagagcggtgaccaactcgctcccgttggtccctatggcaggtgttggaatgaggtgaggctggcctcctctgtttgctcacagctttattcctctagcttcagataaaaataattataataggcaataaagtctcttcacagtgtatttactgtaactacaacagctcgcaagacacattctttttctgcagaattgtagcgggtacttagggagtttgccgaccagaccttctgcacaggaagctggcttgcatcatggaggcagtggactcctgatagtcccgcctgcgcaggcccctttaagacactgacacaaaagactgcccagagtggcatttgctgctgaggctccagggcaaaaaccccatgaacaggaaagcatcctctcagaaacagataatccgcttctcatcttcatagactttggcctcctctggttttatcaggtttccatttaggctgttgaaaaggaggaagacaaaagtacacaggtaaaatgttaaggatcctcgcacactccttcccaaggtgtgttcatagcacacactcgtgtatcacacaatacacatgtatccctgaaattaaggaacataccctttgcagatagttccttcatattagcatggatcctctccagctgccatccctctgttcactgggtctcactaaggtcacctgtagcctccataagcctgaatttaatgacattttccaggtcttgtctttcttggccctcaggattttggctgtcctgacctctcctctccctccttcttgaggcactccctccccttggcttggttatatgcgctgatttttctcctgcatctgagcactctacttcaatttcctttcctatattctcctccatgtgacctctacatctgagatttcctccaggtttggggctaagcccttttccccacccgatctcatcaatgccacggccttgtttgtatgtccgtggaggcagttacctgcttggcatcctcacttggacctctcccccacacctcaaagtagccacacctcctagggtcccctggcttagtgaatggaccactctcatcctgctgagcaggttagtaacctgggggtcaacctggacaccttccttctcatccccatgttccttgtatcacaaagttctcccctaattcctaagaatctagaatgcctccctggaacctctatgtctttggccggccatcttggtctgagcacccatcatctcttccctgcatcagtacaatgtcctactgacaggtcatttccacgtactctggccttctccaattcagtttcctagaagagccaagaattggtctttttgcattgcagatctgatcacatgcctctcctgcttcaaactgttcactgactttccatcacttttaggataaagaccaaaatgcacatcatgacccacagatcttttatggtccagtccctgcctatctcctcagcctcctgtagtcccctcttccacactgtctgaaccctggggcacagcagcttctggcagttcttccaggacaccacactccctcctgccccagaccctttgcacatgctgttccctacgcagaatactcacctcagctgactagctcctattcatccttcatggcttagtgtaagtgctgcttcctcagagaggccttccctgcaacctcatctaaatcaggttccctcttggccttcccctttgtgatagtacccatcacagttgtaattaaataattacgcaaatgattttttctttaatgtgcttctccctttctagacagtgaacccatgaagccatgttgttcaccactggatttttggtgctagcacagtccctgatgctcaagcgtttcgttattcaaggaataaatgaagacacagcactagtccctaggaccttgactcaggccccaccctcccagagccacagtctggaggagatagcctcacagaaagctgttatactggagtgggatacatttctgtaatttacagtgttcattgttcacctctaatttctgttcttaaagacagaactcatcaagggacttcagacttccaagttattttcagaaaggaatgaggtagaaataagtgtcagtacagttaacaagtccaaggagaaggcttactctgtaagtgttggctccaagcagagctgctccctgaacgtggctccccggccagctcaaccacaaccctgtggcctggtgtggcagtccctggcagggccaacaaagccacagccttctcagagcccagtcccatgaagtcacacacagaggagcatgctctggaggctcccagaacaagccagggacctaagtgaagacactcccaagaccagcacccatgccgcctgtcctgccctctctgaattggtggccagttcccctctccaccctgacacaagtgcaggccttgaagctgaagtctcctggccctactaaaatggttccttgtgtaggaatggtgaccctcaagagggaagcaatgcagattttgaaacttgacagaattaaagcaactgtaggtctttactacccgcctgaccagctacagagaaaagtggaagccaactgtgctctgcaaactgttctgtagtctggcagtctccttcctctccccctctgcctgctgtcctcaaaacttaagaatcagcagaaacggagtggcctgagggcaaacacccatgcaaacagttccagtgccctccagtgcttcccaggctgctgggcaggctgggcttcgcaggcacacaggccacaggtgtctgtttagatctgggctctattctctatgatggtgtttttctttttttcttttttgttttttgagacagggtctctggcacccaggctggattgcagtggcatgatcctggctcactgcagccttgatctcccatgcatccatcctcccacatcagcttcctgagtagctgggactacaggcacatagcaccatgtcgggctaatttttgtagtgacggggtttcgcatgttgcccaggctggtctcaaactcctgtgctcaagagatctgcccatcttggcctcccaaagtgctggtattacaggcatgagccaccatgcctagccaacagtgttttttttaaaaaaaaaccttttgtctacaaccctaaataagaattacattttatgttgtgaaccaacacttatgcaaacacacacccactcacatgcaagcatgaactaagatgaatgaaatgatactttcataccatgcacattgcatcctgatattttgttctgttctgttttctttttaaatattgttggttgcaattcaccaaactgatcgattttatgacaagttccaaacaagttctaaagcaaagtttaaaaaacactgtatgaggagcctgtgttggccaaggagctgttttttgtttttttttaattggctatgaaattctccccagtcatgaaaggaggacatagttaataaggcccctctcccagcatccacccagtcccacgtcacccaggcaattaagttgttaagggagctgctgagtggggagtgtctgtgccttatgggaggcaagaaagaagtggcatggcgctcaatgtcacagtaaagcaaactttttttcttctattagcataaggaagttctcaaagcagaaaaaagaagtagacaaagagcacaggtcagttcaagtcagcctggaaatgtcttacggaacccagaaaaggggaagaatgagctccaaactcctggccaacacagtcatctctcaattgctgagtcccagggggatttacttccactaggaaatcatttcactggccacttcccccatcccacccaatgaaggtagatgagggcgggggcccagggcaggagagcacagaatatggaatcaggcattctgggctgaattctagcttctctcgtagtgaccttgggcaaccttatttcactggtctgggcctctgtctacctctgtaaaacagaggtaataccattactgtgcagggttgctggaggaataaacagaaaccaccctgcgttgtgccttgcacagtcagtggtggtgagtaaacagtcactggtgttattactgtgacaaacgctgggatcttaccaaatctctgttatgccagtgttgctctgtaacgcatctgccagctgggcagtccccttagctgtgatctgattctggataagcctgaaagaagaacataacttgtataaaatccataatactggatcaaatttgaccccttcgtaagggagttgcaagggtgttttgggataagagacaagttggtcaggggaagaactgagtccatgtgactttgtatctggctaaaagtgtccaggtaagatccactagagtaaaggatagcttgatattttttaaattttctgtttgtttttatagtgaacataatgtaatacatgaactgatgtgtgtgtttgcatttttcactgcagaggtgcataatcaaaatatttggggccgggtgcagtggctcacacctgtaatcccagcactttgggaggccgaggtgggtggatcacgagctcaggagatcgataccatcctggccaacctgtctctattaaaaatacaaaaaaattagctgggcatggtggtgtgcacctgtactcccagctactcgggagactgaggtaggagaattgcttgaactgggaggtggaggttgcagtgagccaagatcgtgccactgcactccagcctgggtgacagagtgagactccatttatatttttatatatatttgggagaccaccaatctgaacaacaaattgtaagaaacacaccatgggacaggaagtctgacgtctccttccaggtagctagggaaaggcaaggcaggttttaaagttaagaggctaccaacatgcaagagggagggaggagaaacagttatgctgccatgaaatagttctcatgaatagttctcatggggctcagtggaaaagctttaataatggggactctgagaatgcaccccattgttctgggtagcagtgattcaaatgaaacaaagttttcagctatcgctaggaagccaggcctctcctgaaatgctcccttgggcagctacgcttctaacccaaccaggaaataaatgcatcaagaggctgtagtgtctccgcttcatcacagagtcattccctgctcctggctgtttgtagaataggctcctgctaaacatgacccttagaaatcctggcactgacccaagaaggaagcccttaccaggaagacgagggccctatgctgtcatgaatgaagaggactccctcgtgagaggcagtattgcagagcgggtgcagctcctcttaaaccctccctccccactgttcagctacagaaccttgaagaaataaacgcatctgtctgtgcctcagttgtgtcatctgtaaactgagaataactataagagtgccccccatccactgggtgtgtgagatttcaagaggagggtattaggcacagtgtccagggggaagggagctcttgtcactgttactgcccaagcacacaggaccactggtggctgggctgcagccaggatctggtgctcccagccataccccacaagggctagggcccctgggttcaatggcagaggtaatgaccttccctttcaaaagggacaaaaatagaaccaggaagtgggcaccaagttctgtattcagttacactctgctgttttctagtcaccacttaggcttttttgaaggcagggaaggcagcctccaaccaagatgtggcaggaggccaggaaggagagcacctaatgcccctaaaaagcctgcagtgtccaggcacatccacccactcgggctgccttcactagggccagtgcaggttgggaatgcacaaagcagccaagactctctttctgaccctgtcctagctctgctctccctggagaggctggcctgcacttagggaatgggatcaaataattctgcaggctaaaaaggaaagaaaaaacaatatgtagcagagtctagtctttttgcagaaattgacaaggtgagtctgaattcataagaaatgcaaagaacccagaatggtctaaacaatcttgagaaagaagaacaaagtagaactcacacctcctgatttcaaaatgtactacacaaagctacagcaatggtcctggcataggagagacatataaatcaatggaccagaacagagaattcagaaataaacccatacatttatggtcaattgatttttgacaagggtgtcaaggccattcaataggaaaagataatctgttcaacacgtggtgctgccgggacagctggatatccacgtgcaagagaatgaggttaaccctcactttacactatacaaaaattaactcaaaatggattgcagacctatatgtgaggggaaacaactataacgcttaacagaaaacttaggtggagttctttgtgatcttggattaggcaatcatttcttagataagacactgaaagcacaagcaaccaaagaaaaaaatagataaattggacttcatcaaaattaaaaactattggccaggtgcagtggcttttcgcctgtaatcccagcactttgggaggctgaggcaggtggatcacctgaggtcaggagttcaagaccagcctgggcctaaatggtgaaaccccatctctattaaaaatacaaaatgagacaggcatggtggctgatgcctgtaatcccaactactcaggaggctgaggcagaagaatcgcttgaacccaggaggtggaggttgcagagagctgagatcatgccactgcactcctgtctgggtgacaagagcaagactccatctcaaaaaaaacaaaaattaaaaactgttgtcctttaaaggtcactatcaagaaagaggaaaaactccacagaaagaaagaaaatacttgcaaaacctgtatctagtaaaggtctagcacccagaatatatagagaacccctataactcagcattagaagacaaacccaattttaaaatgggcaaaggatatggagacatttctccaaagaagctatacagactcccagtaggcacataaaaagacattcaacattgttgtcatcagggaaatgcaaatcaaaaccacaatattacttcacatccactagtataactagattttaaaagatggacaataataagtgttggtgaggatgtggagaaattggaatccacatacattgctggcgagaatgtaaaatagtacatccactttagaaaacagtttggtggctcctcaaaaagttacagaattgccatatgacccagaaattctattcccaggtatattcccaaaagagatgaaaacatatgttcacccaaaagcatgaacaggagtgttcacagtagcattacgcataatagtcaaaaagtagaaacaacccaaatgtcccatcaactgattaataaactgcaaaatttccttagagtgaattatcatttagccataaaaaggaatgaaacatgctagaacacagagacaaaaggtcacatattgcttgattccatttatatgaaatgcccagagtaggcaaatccatagagacagaaagcggattagtggttgccagggtctggggaggaggacagggagtgactgcttaatggataccagggctttttggagtggggaggaggagtgaaaatgttctagaattaggtggtgatggttgcactaaaaacctctgaattgtatactttaagagagtgaattttacagtatgtaaattttatctcaaaaaatacttttgaaaagtagcacagtctgaaattgtaaggctctctgagttaccataaatgctttaacgtctggttgactttcaacatttctgccaaactctctgccacttcatcgttgagttcattttgggtcagcctaaggaaaaaaaaggaagtatttactcaagagagcctacttggcagacattgttttagagttcacaggagcgggagctcagcccaaggctctccagctatgcagatgatcacttgctgagaacccagaaaacagccaggctccaagcccagcctgtcagccccacagaaggtcaatgggactcatttttaatgcaaatcctgtacctgggctcctatttcttagcccagctacctgtagaggagcccaccctccctccccacctttgctgagccctcagatttcatggaagatgggggcccacggaaaacaaagggccaatgcctgcttctcagttctcctctagtcccagggtcccttgaacaccactgacagtgtgttatccaagttagtttgaagacctgaaacagtcactgactgcacattaaaatgtgttcagtactttgctctcagttaacttgtgagggacccaagttaagtgactgaaagttcaggaagtctctcaatggtccttagaaccagaagtggtctggataaagcaccaaagctccttggtgtaaacagggtaggacccacttgtgccagtggacgaaagaatcattaggaagaagaatagaacagaaagaacatttaaaaaaaaaaattccttgaagggcagtttggatgtgcctgcccaaatttttaatgtgcttcattttaagacaatcagacttctagaaatttaccctacagtgtgcaaggatatacacacaaagctttatacattgcagcatgcattgtggtattatcctaatgtaaaatctgaacaaatttagatcagaaacaactgatgaaatatattgtgatatctattcgagatgttgtgcagcttgtagggagaatgaggtagaatgaggagggagaagaacacagcattgacatctactccggactcagcctgggcaggtttgtatcacccaccagcagggtgacctcgggcaacaggcttaagttctttgggtctgagttttctcactggtgaacgttaacagtagagatcgtatagagtggttgtgaggctgagtgagttaacacagggaaaaagtgtagaataacacatggcatgcagtcagcacttgagggcttctaggtgttgacattactactatccatgtgatttcactctccatggggcacctggccatgtccggaggcatttttgatcctcaccactgggaggtacagggcagagatgcggctaaaccgtctatgatgcacaggacagccctaccataataaagaatcacttggaccaaaatgtcaacagcatcaagatggagaaaccctaaaggagacatgtaaagtatgtgattacaatatttttagagccactgcttgaatgctattctgttttgcatatctttgaacatacatagaacaaaaggctagaaaggaaaggttattttggaaaacacaaagggaggtactttcacttcctccttttagcaggaactttaggaggtactttcatgtcctcctttagtgtcttacattttttccaaaaaacataaactacttttcaaaatgttaaatgatttgttgaggggaaagaatttaaaatgcacttactggacagcaggcctaatgtcaaaactctctcatgaagaggatattgtcattattctcagtaactaatttagcaagatcaaatgagcactgggctctgagtagatgatagggagggttgtaatacaactccctcacttaacctgtaaggaatgttttttttttttgtagatagagggtctcgctatattgcccaggctagtctcgaactcttggcctcaagcgatcctcccacctcagccttccaaagtactgggattacagatgtgagtcactgctcccagccacctgtatggaatttatgatacagagatgtaaatacaaataaagcaaaataaaacattaagtggccataaaaaagggaacaccaacaaaaaacactctcaggaggccacatccacttagtaagtgccctagagtgcagagagggagcgaggactgtgggcttcaggtgacaagtgacagatgagctggatggagcataaaggccaaaccctgatgtgaagacctggggaaggaggaggctgcctgggggaaggcagatgaagaggaggaagggagaaaattttgagccaagtgtgtggaagttggcctctgctaagcacatctgaaggtatccaaggatacctggatagcttgcatggccagcgttctagttctcctggggcctagaaggccaaggcctcagatccaggctcccggagcccacttcctcctcctgtacctgtttatgcttgtagaagtggctattctcttctctaggaggaaattatcaacttaactgaaaggcctagagcctttccaattgggagacaccttttctgttaagattccaggccatgtgcagcccctgggataattcctgggacctaaaccctccaaaatgactctgtgccaggtgcagtggctcacacctgcaatcccagggctttgagaagccagggcaggagaactgcttgagcccaggagttcaagaccagcctgggtaacatagcaaaaccccatctctacaaaaaattttaaaattaactgggtgtggtggtgcatgcctatagtctcaggactcggaaggccaaggcgggaggttcgaatgagcctgggaggtggaggctgcagtgagctgagatcacaccaatgcactcaagcctggacgacaaagcaagaccctatcacagaaaaacaaacaaacaaatgaaatgactcgagctattaccacagtatttctagagacgtgttctgctgcagggccctcgcaaggctctttcctccttctgtggagatgccgttggacgcaagactaggaaggaacacacttggggtgaattattaaagacacatctacattcaatcttagcttcaacatcagaacagctgggaagcctctgcctggctgtggccccagcccaggctcactcggggcaggatgcagaagtgttcagctgcagcagtagtgcccaggcgctgctgccccagagcccacacacagagcacaagggcaggctggacgggaaatgggtcatcagcacagcctgctcacacaacagtgggctggggtcggcacctcaggtttctgcacatgggcaggaggctggcgaggcctggcaagctgtccttctaaaggctggccccatcaaggtaccccatctgcaagacctggcaaagtctcttctgctactgtccccaccttcttggactctttttcagtggtaggaagaacagcagcagagccgtggacataccaaggtcccaagatttggccagcagcctaagcgggggctgggtaggtgcatccccatttatagagaggaagagtccctcttttccagcatcatcctgaggacaaggaaggggcaggaatctgctggccagcatcttattaagtttcatctgcacttaggaaaccctccctctaccagcaagggacatgcagggatggctgaggggctggaaatgtcctctgtcttgatgggggtggtggttggctggtgtgggacacatgcagaattcactgagctgaataaacacttaaggtgattcctcagggatctagaaccagaaataccatttgacccagcaatcccattactgggtatttacccaaaggattataaatcattctgctataaagacacaagcacatgtatgtttattgtagcactatttacaatagcaaagacttggaaccaacccaaatgcccatcagtgatagactggataaagaaaatgtggcacatacacactatggaatactatgcaaccataaaaaaaaatgagatcatgtcctttgctggcacatggatgaagctggaaaccatcattctcagcaaactaacacaggaacaaaaaaccaaacactgcatgttctcactcgtaagtgggagttgaacaatgagaacacatggacacatggaggggaacaacacacaccagggcctgttgggggtggggggcaaggggagggcaagcattaggacaaatacctaatgcatgcagggcttaaaacctagatgacaggttgataggtgcagcaaatcaccatggcatgcatttgcctatgtaacaaacctgcacatcttctacatgtatcccagaacttaaagttaaaaaaagagaagaatgtgctttactagatggcctcttatgcatcaaagaaaggtttgacaagagtaaggaaaatcacacatgcaaattcctcttctattaggagcacgaatcacccttccccagcaaacctagagcagctgggagggaccaggttggcccctggagacagcagggccacagttacctcagggtggtcaagctggggtggttccgcagagcctctgcgaaggcttttgctccttcatccccaacttgattgccccacatcctgagggaggagacaagatagtgaatgttagctccccaagaaacagccatgctcacccctccttttttgccaatggccacagctcagttcctactcaacaggcagatgtttgtgcccagtccctggatctgtggcctggacactaattcctcacactggtctctgagtgggctgctctggatttcaaattcaaggggtgatgcaagatcgaagagcaggggaagccttgtgaacagaggccatcggcaagtggcacagttagagaccgagcctcaccacgagcttcggagagctgtcagcccaggagcctgagctcatgagagaaagcacctgcatgtctcctgtggagcacagaggtgcgcagagtcctggagggaaggccgtgtgcaggtgctgggttcccagcatccgttcaactctgggcagggtcagagacattcctgaagacacagtcttccaggagacaaagagcaggaagtaccttcagaaaccccatcagcaacgttcctgtcacataaagaggacgttacccaaccaagctctgtggctacggccatcttggtggacaggtgcggcgataaagctgtattctgtcccctacagcagatccggagagtctctggctaaaacagtctgcgtggtaagatggtggactcctgacccggctctgccatctgctacctgtgtgaacttcgggaaattatgtagtctcttggacctcagttgccccatctataaaatggggataattagagcattccgtcatggagttgtgaggattcactgagttaacagaagcaaagcacttagaaccgtgtgtggctcacacaaagcgttggctgttcctaataggatcagcacacactgtctacacacaacagaacaagttcgtgtggcccactcacaacacaatggcagctgcctcaacaacacgttcacaccaagcaaactacctgcttacaccattccaggtatggcttgctggaacccagaataagagttttcagtgttttgcttacatatatgtatttactatacaattgaatggcatcacatttttaataaagtggaaagctactcaaggcatgagattaagtgaaaaaggaaggatacatatagtctcacccctactatagtatagatacatatagtctcacccctactatattttttaaaagttgtacaaagagagagaagggagagaaagtgagagagcttaactgagcttaaatgttaacaagaatcattttctgagaagataatttttattatttttcatagtttccaaataaaattacctgtgttccttttgtaatccaataaaaattataatgaaacaaaaagctttaaaaagcctcaagaatcaaaaccacaatgagacaggacctcacactcattaggatgggtactgtcaaaaagagaaaatagcaagtgttggcaaggatgtggagaaactagaatgaactgttggtgggaacataaaatggtgctgctgccgtggaaaacagtatgatgattcttcaaaaaattaaaaattaccatgtgatccagcaatcccactgctggatatatacccaaaagaattggagggaggagccaagatggccgaataggaacagctccggtctacagctcccagcgtgagcgacgcagaagacgggtgatttctgcatttccatctgaggtaccgggttcatctcactagggagcgccagacagtgggcgcaggccagtgtgtgcgcgcaccgtgcgcgagccgaagcagggcgaggcattgcctcacctgggaagcgcaagggatcagggagttccctttccgagtcaaagaaaggggtgacggacgcacctggaaaatcgggtcactcccacccgaatattgcgcttttcagaccggcttaagaaacggcgcaccacgagactatatcccacacctggctcagagggtcctacgcccacggaatctcgctgattgctagcacagcagtctgagatcaaactgcaaggcggcaacgaggctgggggaggggcgcccgccatagcccaggcttgcttaggtaaacaaagcagccgggaagctcgaactgggtggagcccaccacagctcaaggaggcctgcctgcctctgtaggctccacctctgggggcagggcacagacaaacaaaaagacagcagtaacctctgcagacttaagtgtccctgtctgacagctttgaagagagcagtggttctcccagcacgcagctggagatctgagaacgggcagactgcctcctcaagtgggtccctgacccctgacccccgagcagcctaactgggaggcaccccccagcagaggcacactgacacctcacacggcagggtattccaacagacctgcagctgagggtcctgtctgttagaaggaaaactaacaaccagaaaggacatctacactgaaaacccatctgtacatcaccatcatcaaagaccaaaagtagataaaaccacaaagatggggaaaaaacagaacagaaaaactggaaactctaaaacgcagagcgcctctcctcctccaaaggaacgcagttcctcaccagcaacagaacaaagctggatggagaatgattttgacgagctgagagaagaaggcttcagacgatcaaattactctgagctacgggaggacattcaaaccaaaggcaaagaagttgaaaactttgaaaaaaatttagaagaatgtataactagaataaccaatacagagaagtgcttaaaggagctgatggagctgaaaaccaaggctcgagaactacgtgaagaatgcagaagcctcaggagccgatgcgatcaactggaagaaagggtatcagcaatggaagatgaaatgaatgaaatgaagcgagaagggaagtttagagaaaaaagaataaaaagaaatgagcaaagcctccaagaaatatgggactatgtgaaaagaccaaatctacgtctgattggtgtacctgaaagtgatgtggagaatggaaccaagttggaaaacactctgcaggatattatccaggagaacttccccaatctagcaaggcaggccaacgttcagattcaggaaatacagagaacgccacaaagatactcctcgagaagagcaactccaagacacataattgtcagattcaccaaagttgaaatgaaggaaaaaatgttaagggcagccagagagaaagttcgggttaccctcaaaggaaagcccatcagactaacagcggatctctcggcagaaaccctacaagccagaagagagtgggggccaatattcaacattcttaaagaaaagaattttcaacccagaatttcatatccagccaaactaagcttcataagtgaaggagaaataaaatactttatagacaagcaaatgctgagagattttgtcaccaccaggcctgccctaaaagagctcctgaaggaagcggtaaacatggaaaggaacaaccggtaccagccgctgcaaaatcatgccaaaatgtaaagaccatcgagactaggaagaaactgcatcaactaatgagcaaaatcgccagctaacatcataatgacaggatcaaattcacacataacaatattaactttaaatataaatggactaaattctgcaattaaaagacacagactggcaagttggataaagagtcaagacccatcagtgtgctgtattcaggaaacccatctcacgtgcagagacacacataggctcaaaataaaaggatggaggaagatctaccaagccaatggaaaacaaaaaaaggcaggggttgcaatcctagtctctgataaaacagactttaaaccaacaaagatcaaaagagacaaagaaggccattacataatggtaaagggatcaattcaacaagaggagctaactatcctaaatatttatgcacccaatacaggagcacccagattcataaagcaagtcctcagtgacctacaaagagacttagactcccacacattaataatgggagactttaacaccccactgtcaacattagacagatcaatgagacagaaagtcaacaaggatacccaggaattgaactcagctctgcaccaagcagacctaatagacatctacagaactctccaccccaaatcaacagaatatacatttttttcagcaccacaccacacctattccaaaattgaccacatagttggaagtaaagctctcctcagcaaatgtaaaagaacagaaattataacaaactatctctcagaccacagtgcaatcaaactagaactcaggattaagaatctcactcaaagccgctcaactacatggaaactgaacaacctgctcctgaatgactactgggtacataacgaaatgaaggcagaaataaagatgttctttgaaaccaacgagaacaaagacaccacataccagaatctctgggacgcattcaaagcagtgtgtagagggaaatttatagcactaaatgcctacaagagaaagcaggaaagatccaaaattgacaccctaacatcacaattaaaagaactagaaaagcaagagcaaacacattcaaaagctagcagaaggcaagaaataactaaaatcagagcagaactgaaggaaatagagacacaaaaaacccttcaaaaaatcaatgaatccaggagctggtttttcgaaaggatcaacaaaattgatagaccgctagcaagactaataaagaaaaaaagagagaagaatcaaatagacacaataaaaaatgataaaggggatatcaccaccgatcccacagaaatacaaactaccatcagagaatactacaaacacctctacgcaaataaactagaaaatctagaagaaatggatacattcctcgacacatacactctcccaagactaaaccaggaagaagttgaatctctgaatagaccaataacaggctctgaaattgtggcaataatcaatagtttaccaaccaaaaagagtccaggaccagatggattcacagccgaattctaccagaggtacaaggaggaactggtaccattccttctgaaactattccaatcaatagaaaaagagggaatcctccctaactcattttatgaggccagcatcattctgataccaaagccgggcagagacacaaccaaaaaagagaattttagaccaatatccttgatgaacattgatgcaaaaatcctcaataaaatactggcaaaccgaatccagcagcacatcaaaaagcttatccaccatgatcaagtgggcttcatccctgggatgcaaggctggttcaatatacgcaaatcaataaatgtaatccagcatataaacagagccaaagacaaaaaccacatgattatctcaatagatgcagaaaaagcctttgacaaaattcaacaacccttcatgctaaaaactctcaataaattaggtattgatgggacgtatttcaaaataataagagctatctatgacaaacccacagccaatatcatactgaatgggcaaaaactggaagcattccctttgaaaactggcacaagacagggatgccctctctcaccgctcctattcaacatagtgttggaagttctggccagggcaatcaggcaggagaaggaaataaagggtattcaattaggaaaagaggaagtcaaattgtccctgtttgcagacgacatgattgtttatctagaaaaccccatcgtctcagcccaaaatctccttaagctgataagcaacttcagcaaagtctcaggatacaaaatcaatgtacaaaaatcacaagcattcttatacaccaacaacagacaaacagagagccaaatcatgggtgaactcccattcacaattgcttcaaagagaataaaatacctaggaatccaacttacaagggatgtgaaggacctcttcaaggagaactacaaaccactgctcaaggaaataaaagaggacacaaacaaatggaagaacattccatgctcatgggtaggaagaatcaatatcgtgaaaatggccatactgcccaaggtaatttacagattcaatgccatccccatcaagctaccaatgactttcttcacagaattggaaaaaactactttaaagttcatatggaaccaaaaaagagcccgcatcgccaagtcaatcctaagccaaaagaacaaagctggaggcatcacactacctgacttcaaactatactacaaggctacagtaaccaaaacagcatggtactggtaccaaaacagagatatagatcaatggaacagaacagagccctcagaaataatgccgcatatctacaactatctgatctttgacaaacctgagaaaaacaagcaatggggaaaggattccctatttaataaatggtgctgggaaaactggctagccatatgtagaaagctgaaactggatcccttccttacaccttatacaaaaatcaattcaagatggattaaagatttaaacgttaaacctaaaaccataaaaaccctagaagaaaacctaggcattaccattcaggacataggcgtgggcaaggacttcatgtccaaaacaccaaaagcaatggcaacaaaagacaaaattgacaaatgggatctaattaaactaaagagcttctgcacagcaaaagaaactaccatcagagtgaacaggcaacctacaacatgggagaaaattttcgcaacctactcatctgacaaagggctaatatccagaatctacaatgaactcaaacaaatttacaagaaaaaaacaaacaaccccatcaaaaagtgggcgaaggacatgaacagacacttctcaaaagaagacatttatgcagccaaaaaacacatgaagaaatgctcatcatcactggccatcagagaaatgcaaatcaaaaccactatgagatatcatctcacaccagttagaatggcaatcattaaaaagtcaggaaacagcaggtgctggagaggatgcggagaaataggaacacttttacactgttggtgggactgtaaactagttcaaccattgtggaagtcagtgtggcgattcctcagggatctagaactagaaataccatttgacccagccatcccattactgggtatatacccaaatgagtataaatcatgctgctataaagacacatgcacacgtatgtttattgtggcactattcacaatagcaaagacttggaaccaacccaaatgtccaacaatgatagactggattaagaaaatgtggcacatatacaccatggaatactatgcagccataaaaaatgatgagttcatatcctttgtagggacatggatgaaattggaaaccatcattctcagtaaactatcgcaagaacaaaaaaccaaacaccgcatattctcactcataggtgggaattgaacaatgagatcacatggacacaggaaggggaatatcacactctggggactgtggtggggtcgggggaggggggagggatagcattgggagatatacctaatgctagatgacacattagtgggtgcagcgcaccagcatggcacatgtatacatatgtaactaacctgcacaatgtgcacatgtaccctaaaacttagagtataataaaaaaaaaaagaatctaaaaaaaaaaaaaaaaaaaaaaaagaattgaaggcagtccttgaagagacatttgtatgcccatgtgcacagcagcattattcacaacagccaaagcatagaagcaagccagtgtctgttcttggatgaatgagtaaacaaaatgtggcatagccatgcaatggagtattactcagccttaaaaaagaaggaaatcctggcggggcacggtggctcacgcctataatcctgacactttgggagaccgaggtgggtggatcatttgaggtcaggagtttgagaccagcctggccaacatggtgaaaccccgcctctactaaaaatacaaaaattagctgggattggtggcgggtgcctataatcccagctactcgtgtggctgaggcgagagaatcgcttgaacccagaaggcagaggttgcagtgagccgagatcatgccactgcactccagcctgggcaacagagcgagactctgtcaaaaaaaaaaaaaaaaaaaaaaaaaaaggaaatcctgtcccatgcttccatgtggacgaatcttgaggacatgatgctagtgaaatcagccatccccaaaaggcaaatactgtatgattccacttacatgaggtagagtagtcaaaaccatagagagagtaagtagcagggtggctgcaagaggcaggggggagggggaccggggagttattgcttaatgggcacggtttcagtttcgcagcatggtgaaagctctccactatctctccccgacagcgagccccagtggtccttctggtgtactgatgtatgaaaagaacagcaaggcccgccccccacacacacagcaggttgtaccacatacatccatccccttctactcacccaacctcagagattgatttgctgttcttcacagccagggcgagatacttccctccttcacttgttattttgttttttcccagtctgcagagagagtcacacacagtcagcctccagctatgcaggggctccaatgtgggtcaccctcctccccagcacagagccccttccagccccatcttgggaaccgtacactttcttctgggatccacaagagccatggcctgggcctcctgtcagggagggaactgaagccctgaggacagtcagtcagggttcctctccaggagatggtccacagtctctgcagaagctcagggacctgagccctccctggggtcctctaattgacctcagcagcatcactgacttccctcgggagacctgggaggtcccactttgactgccaagggtcaaggtatgtggccactgtcagggcaggaccagaggctccaagcctggcgcactgtcaccccctgcgcagggcacacctacagcagggcaagggcccaagctcttccttgcaacccgacaggaaactcccttggggcaaaaagaccaccagaaagggtgagagttagtcctttcttcggcctagtagttaatgaaatcaaaatgccaacctttccccctaccaataacgggaggcccttccagaatccaagcctcacagatcacacagaggtatctttacttgaccaacatggccctgggacaagtggaaggctttaggatgaaagctctttactgtgacatttaatctttgaacaacagaagggggtgatcaagagaatatactaaggaacctggtgcctaccccacttacttaagatgcgtgaggcctttgcattcatccaggattttggtgacgtacctggctccgacatcggtgatctggttgttgtataaacttcaagagaaagcacagccatgagactttctggctcctgatgtcattttaatagcccctgttttttgaagcatatggtgtctactgatttccaaagtctgggttgaaaggggaacacacaggcaccagagtttagggctctgaggtctcacagctcaggttcaaaaccagctccactactttccccagctttatggcctgagacaagtcacgttacctctctgtgcctcagtttctccatctgtaatacagaggtaatgatgatagtccccttgcagggtggttatgaggatcttaggacataacgcaaagccctggtgcatgagtgctcggtgactggcaggtgatgggtatttacaactaagattacattcagcgtcttgagaagaaaagattctgaatattaaaacttctcgatgtctacctttcagccatctcaccgctcaatgccaattcaatagtacagcagatggaagttgaaaggcagctaagctcaggcttccagggttccacagccacacagagccctggagccgagtgtggcccggggtcgtttgcgtccatgtcctcattaacatcgaaggccactgaggccccaagaaactgaacaactgctccagcttgcagagccaccagttgtgtggggctggcccgcaatcccttctctcagactccaaaacctcaaaggctctgaatgcccaaagtttctcacaattcttttggcaactgatgtggagctacttatagtctttcttaccacacttggtttgactgtttcatacattctgcgggagaaatatgaaagtgtttgaaaatggggcactgccttagacccctgcggccacgtcatgccatagcatgcactcagaatccccattcgggtcttgccccaaaggctctggatgagggattatggacccaaaatagtccagactaatatgtaaacaggattctagcatttattaaatatatccccttatattctcgttgcatcccccaataaccagttgtaagcctacttgcctatactgggatcccagttttacaggtgaggaggctgaggctcagcaagacttcatgactggccgagccacaaagagaacccaggactctcaactcctgggccagcgctcttcccagccctgggccatccgtgccgatcatccagggacctatgggaatgtgaattccctgcagctctgtattattactaggtagttggcccagtgttctggagaaagacataccccaaataggtcacaattttgtatttggtcagctcttcgcttagcacctttaccccaccgtcagtgatctggtttacgctgagtctgaaataaaacagcaaaaaaattacgagtcagggcaggcactcattatgaaggaccattaatcttacaaagatccagaagccttcaggccctggagttttctcaggtatagacgcccctggagaatggccacagcaatcagccaaatagccccttacaaagcctggccaacgtggggccgccagacccatcctgcaccagcccatgagcgaggctgcagcacagcccctggctcctcacacgaccgtgaaggacacagggaggacgtgtgaggccagccacctctgacatccaccctttgcgtgtgacctatgtgtcagccagaccctgtacctggttctcgtatggtggagggcatgggtttggaaatgtcaggggctgcttttagtggttgtgactggaggagggagtgccctgtcatcttgcaggcagggccaatgatgttaggtgtcctgcaaaatgctggacaggtctcacaagaactgttatacccaaaatgccaacagcttccccccatccagaaacacatgggctaaccctgatttgtcactggaaaaggctgtgagtcccactgtcaaaggagactgctcatgtcattctgtaaagtgtcagacagcaaatattttaggctctgtgggccatgtggtctctgtcacagctactcaactgtgtagtgtgaagcagccagtaagtaaataaatgggtatggctgcgttccaataaaactttacttagaaacacaagccttgggcccaattagacccacaagctgaccttgataacaagtaaaaggctgaacaattaaaagcttgatttttaggccacatcaaaatgatcaagtcagacactctggaccctttccctcagagtccccagcttggtgctgggaaatagtgtgtatgtacatggcatggccagcccaagacagcacaggctgctagacattcccaatatctatactttcttagcaatagattcccccatcttttagctgggcccatctcattaaattctgcccataccattaagttctgaaatctaagcagcaatataattagtaatagccaagaagtgttcttaaaagattgaggcatgctctcaatctttgcccttccttcttactgatagctagagtgtcgactagatggctggagtttgagcagccctcttgggccaagatgggaaagccgggtagtgaggatggcagaacaggaaggcagaagagagcctgggctccggagggtcatggagactccataccagtcctgaactacctacctctagacttttacatgagagaaataagcttgtaactcaccaacatttggggttttcgctctcagctaaaactaatcctaactgatgcagccagagaactgagtgtgcttcccgtggttacccatatcctctgctttcctacaagctgttttttcttgcactggtgtgaaggaaagcttcacctggggacagcaggaagggcagggccaccacactggtgtggagtttcccattgaaaagatggacagaggggtcgggtgcggcggctcatgcctgtaaccccaacattttggaaggccgaggcgggcggatcacctgaggtcaggatttcaagaccagcctggccaacatggggaaaccccgtcctactaaaaatacaaaaaacttagccggacatggtggcgtgtgcctgtaatcccagctactcaggaggctggggcaagagaatcgcttgaacccaggaggcggaggttgcagtgagctgagatcacgccactgcactccagcctgggcaacagagcgggactccatttgggggagaaaaaaaaaagatggacagagggtggcactgagtgtgagactgcctggggctacagagggagagtcactggctgagagtacagagaacagactccaggttttccaattcccaggctagggcagccttcgaccccctgcaccccatgtccccatgcaattatttacactctgacactggtgtgacaggcactgggagtcagttccaaatccctactgctttttagaaatgcatgagtcaagtaatggcaaatttaaccccctgtagagatttacaaggtttattttacataggaaatgctaatagaagaaaacagactgggctctgtttcccgtaaactatggaacaggaaaacaaccacgagtcagcattttaactgaggtctctagcagtgtgaagggctgctagcgttgatcacagaaagtcaccagtgccaactggcacaaggtctgctttctctgcagtaataaaagctcccatattccagaaactttttgagggctatctcggagccttacattaatcttaaaaggaaggttctttatgcattctggcactgctatgtctgagctgtgtgacctaaagcaagctattaacttctctgtgcctctacttccttatctgccaaaatagagacaataacagtacctaagttacagggttgtaaagagcattccattaggtaatatttgtaatgcacttcacccattctcaactaacattggtccctgtggccataattgataggattatgagcatcttctttttccagatgagggcgtggaggctctgagagttaagcactttgttcaagggcacccagctgtggctgatccaggtccacctgcctcgaagctttgcaccttgacctctgccctgctaagaaagaaaaggtctggacattccaagggccatggtcatgagtcctgggggatcctggtccatgatgccattcccgatgccctccgagcctggcccgcccggcccacctgttgctccccttgcctggcagcctcacctgagaacagtgaggcggctgaagcagggctgcagctcccgcacgccgtagtcgttgagattgttgttgtctaggtctagggccagccgcttggggaagtgatgcaggacgaaggagagggcgctgcagtcggccgagcaggcgttgcagtaggtcagcttgaggtagttggcgcagatgcccctggccgccagctgccccaccttctggctctgtgtctcgtagatgcagcgcagcatccagatgaacgtgggcatggcctgcacctggttgaagctttcgacctgaacgcggggcaggctcttcaggtagccccgcaggctggaaaacaggtgtgcccacagggccttgcgctttctcctcagggctgccgcgggcaccagatgccgcaggagtttctgtttggctttggacaacagcccgcacaggaagaggttggtgaactggaagtgatccttgttcttgaagaggtcttcccgcgccggaccactgccctgcaggcactggaacgggaggaagggaggatagcaggacgtggtcgctgcccccgcagggggcatccactcctggaagaacctgagcagctcctgagtgcccaccctgtcgtccagcacgaggaagaaggctgtaaagaaggcctggagggtgaggtggaaaaactcataggactgctggtcacccccggggcccagctccgcaaagcccgcaggaagcccagctgcatgtctctctcctgcagcccggagcctgcacctcctcctgggtgaagacaaagaggctcttctccatgccccggtgggccacctgccccagcgagcacagagtgtcccggccggcgtggagggtctccactgggctgcgtgtgttccgctgcaccaggctgctgggctgcatcctgttcagatggacctcagtgaccaggaggaagacatctgtcagggtcatcgtgcagtcgggcagctgtggtgagccttcaaaggcagcacggaagtgctggaagcaccggaagatgatccagcagaagaggggcacagagcacaggctgcagaggttggggttggcctccagctggctcagcaggcggtcctgcagggcccgctcggggaacatcctcctggcataggcgcgcaggtggctgggggagaagccccggagaagcaccttcttccgcaggaactggcgcgggacctcgatgcctgtgcgggctgtgagcagcttgctagcccccttgagcagcttcccactgagcaggttggccagcaagaccagggggtgggcaggctcccaggggcaggagctgtcaggcacgcggctcaggtccaagtccgagtgcagctcgtccaggccatcgaaggtgaagagggccacgtgggggaagcgcagcaggaaggcaaacacctcctcggggtcccgctctgggtagcagtagtgcttgaagagcaggtcctgcagacacagcctgtcactttccttgaagcagctgaacatgcggcagcgaaagtggaagaagaatttgacccctgcgtctagccggcccgtggcccagaggctctgcagccgctgtagcagcatggacttgcccaccccagcatcacccaggatgaagatggtctcaccctgctcattgaggatgccggtggtgtggtccaggaggcaggccaggctgttcaggctgcccaggctctcattgctgaagccaaccagctccatgatggtgtccatgtagatctcctccagcagcagctcctccttctgggcatagcacagcacgaacttggagtcacggcccagatggtgtcgcagctgctgggtatacctgctcactggagggagggggtggcagggcacagcagcagggtgaggagagaggcagaaacagcatcaaacagggaggtctcaaagaggagaggcgagtgcataaacaaaattcacaacctgggccctgaggccagcgcagccactcctcagatgcaggacctggggagcgtcacagagccagcattcctgaccctgagggccccaggctttgatctctctgcttgacctcagatttgccagaaagaaggtgacaaacttcagctgggatcagtgtctgagacaggccacccttattcctccgcacagtgtcatcactgaactgcagctaaacactgaagcccctcgcactcgagggccaggccagatggccacgctggccctcctccaccaacactgcttttttgttgttagagatagggtcctgctctgtcactcaggctagggtgcagtgacccaatcatgcagtctcaaactcccaggctcaagcaatcctcccacctcagcctcccaagtagctgggacacaggcatatgccaccaagcccaactaatttttgtgtttttttgtagagacggggtctcgccatgttgcccaggctggtctcaaattcctgggttcaagcgatctgcccacctcggcctcccaaagtgttagggagtgttatatatttgtcagtaatttttagtaagtttacaaagttgtgcaaacctcaccacagtccaggtctagaacatctctaccatcttcccagagttttctggagcctatttgtagccaatccctgggccgtaggcaactactgatttgctttgtctatagatttaccttttctggacatttcatataaacagaatcatacaatatgtagtcttttgtgtgagactgcttgtagagtgaattacactgtaaaagctagtaaagtagctggctaacggaaccaacagagaagtctttcgaaagaatgattcctcatgggaatactttctttttttttcatgggggaaggaaacaaggggagacaggaaggagcagacagagtcagaagctagcagtggctggcaggcacctgcaggggctgtggaagctggagcctagcactgacactctgccggctggccttgtttcccctaagcacacacctgtcttgtgcttctgtttccaacaacatattggattctttaaaaggcaggacttaggtccttggtgctggtatgttaccaacagctgtctcaaagtggagagttcaaaagcagggaaagaagactcctgccccagagcagatcctggccctaatgatatgttctcacatgccttggactgccgctgtcctgacacatttttgagattgctgactggtggtctcttccagctgacttgaagctccctgagggcaggaaccctgtctgtgtctgctcaccactgtctccctgacatccagcacagtgtagggtcttggcatgtggtggatacagcaaaaaatacttattgaactaatcagttaactgatggggtcttccattagtttggaaataaggactggcactcaacggtgacagaatactttgcctagaatcaaacctggcaaggctttttaatttttttctttccttttttagagacagggtctcactctgtcacccaggctggagtgtaggggcacaatcatagctcgctgcagccttagactctggggctcaagcagtcctcttgcctcagcctcctgagtagctggaactacaggtgtgtgctaccatgtccagctaaagatttttttaaaagaaaaaaaaatacaaccttgaatttgtgcaaccattaaaatgtctacatatgtcagaataggaagaaagcaaggggtcagaacacaggccattcccatacttccaaaggcccctctataaaccagaccatcgaggtgtgggcagttcaatcgcagccacctgggctgcacttgggagacaggattcagctgttcctttctaaaatcaaaatagcacctgggcctgcttcctgaggtggcactcagggctggcccagccattaccaaaccccccagggccctgcttgcactggtgcctgcggtcttgctggggctgactcctacctgggtcagtgttgaccacgactttgctctgagtgagcagggaaggggagaagccgatctccagcagccaaggcctgaggtccacgtaggcatctgcgagttgctggagcaagtagaggaagaactcggacacctcctcgcccttgctctgtaccaggtccagaattttgcggacctggaggtgacacaagcatgagggcacgggctggcatgaggggcatcctcctaccataaggaccctcaggcaggctcagggcagggctctgagttcttagtacagccggtagctatgccatccccccagctgctgtgacagtcaacatattggagcccgaaaatctgtgaaatgacctcctgagcaagaaataaaaccactcagctctaagaaaccaggaggcttccatgagcctaaaaataaaaaggacagacaaatagccatggacttctctacctcttttttttttttttcttttttgagacagagtcttgctctgtcacccaggctggagggctggagtgcaatggcacaatcttggctcactgcaacctccacctcccaggttcaaggaattctctttcctcagcctcccaagtggctgggattacaggcacccaccaccatgcccgactaatttttgtgtttttattagagacgggttttccccatgttggccaggctggtctcaaactcctgaccttaggtgatctgcctgcctcggcctcccaaagtgctgggattacaggcgtgagccacggtgcccagccttctccttgtccctcttaacctgacacaactgctgtgcaggaatgagagtggagaggtgagaaccaccccttgggcataatgaggtaggcacctgggccacacagtaggagggcgataggccatatattaaaaacggctttttttcttgttaaaaatgtgggaaaacaaatcaagagaagagaacaaagcacacgagtgtgctactgcttcccccagaaaccgtcctggtctcttctgagtcccagcggggtcatgtcataaatgagttgtgtcattcctctaaccaagaaggaatttgtggtctccattttatttttcaggagataacgcaaagcgtaagtggaagtaagggctgttaaggcgtcccacagtggagcggggaagattcttcactgtggacccaccgtgggatgtcatcattcctgaccctgagtcggttcccctccgagtctcacccagcaacaagcagccctccagccttcagaggcactgaatgaacatcgctgctggggatgctcttatgcttatgttctttttccctcctaatttaaaggggatctaaaaatgaagtgtctgggaaccaatacacacagcaatgagaactgagatgtcagggtttgctccagacacaaacacagaagtaaaatacctgactgtgcaacctgctaacccaggcatttgtgtgctattagtaccccaaaatgcagccctgcccgctggactaaactcccacgctcttcactcagatcagcagggagaggcctcttctagctcccggggtccacacaatgccatgcccgtccctgtccccggggcaccttgtcaggctgggtggggcaggcacacacaatctccgcatcttcggccgagaagtagtcattcttcagcaagttgtccaccagacactgagtattgcggatgtgagtgaccagaagttcccgattgcttttcagtaattgaatgtgggggtgagactctgatgggattatttccatctcactgtggccctgctcttccatagttaaagtagcaagcggctacttttcccaaattcatcttcagctgcgtgtgtcctctcagcagaagggcaatcaggattcaggccgcgccctccagggcccctgctactctgcgcagcccctgaagagatcaatgacatcatcagcaaagcaaaagctacagaaagagctcaccccacctcccactggttgtggggtttctctacctagagcaggaaggaaccatgcttaagacctttatctggccaggcaagttggctcatgactgtaatcccagcgctttgggaggctgagatgggtagatagcttgaagtaaggccagtagtttgagaccaacctgtgcaacatagtgagacacccatctctacaaaaataaaaaaaattagccaggcatggtggcgcacctgtagtcccagctattggggaggctaaggcaggaggatcacatgagcacaagagttcaaggctgcagtgaacgatgatc

SEQ ID NO: 2 - DNA, 4093 bp, Homo sapiens
  CDS (130)...(1650)
ctcttcaggggctgcgcagagtagcaggggccctggagggcgcggcctgaatcctgattgcccttctgctgagaggacacacgcagctgaagatgaatttgggaaaagtagccgcttgctactttaactatggaagagcagggccacagtgagatggaaataatcccatcagagtctcacccccacattcaattactgaaaagcaatcgggaacttctggtcactcacatccgcaatactcagtgtctggtggacaacttgctgaagaatgactacttctcggccgaagatgcggagattgtgtgtgcctgccccacccagcctgacaaggtccgcaaaattctggacctggtacagagcaagggcgaggaggtgtccgagttcttcctctacttgctccagcaactcgcagatgcctacgtggacctcaggccttggctgctggagatcggcttctccccttccctgctcactcagagcaaagtcgtggtcaacactgacccagtgagcaggtatacccagcagctgcgacaccatctgggccgtgactccaagttcgtgctgtgctatgcccagaaggaggagctgctgctggaggagatctacatggacaccatcatggagctggttggcttcagcaatgagagcctgggcagcctgaacagcctggcctgcctcctggaccacaccaccggcatcctcaatgagcagggtgagaccatcttcatcctgggtgatgctggggtgggcaagtccatgctgctacagcggctgcagagcctctgggccacgggccggctagacgcaggggtcaaattcttcttccactttcgctgccgcatgttcagctgcttcaaggaaagtgacaggctgtgtctgcaggacctgctcttcaagcactactgctacccagagcgggaccccgaggaggtgtttgccttcctgctgcgcttcccccacgtggccctcttcaccttcgatggcctggacgagctgcactcggacttggacctgagccgcgtgcctgacagctcctgcccctgggagcctgcccaccccctggtcttgctggccaacctgctcagtgggaagctgctcaagggggctagcaagctgctcacagcccgcacaggcatcgaggtcccgcgccagttcctgcggaagaaggtgcttctccggggcttctcccccagccacctgcgcgcctatgccaggaggatgttccccgagcgggccctgcaggaccgcctgctgagccagctggaggccaaccccaacctctgcagcctgtgctctgtgcccctcttctgctggatcatcttccggtgcttccagcacttccgtgctgcctttgaaggctcaccacagctgcccgactgcacgatgaccctgacagatgtcttcctcctggtcactgaggtccatctgaacaggatgcagcccagcagcctggtgcagcggaacacacgcagcccagtggagaccctccacgccggccgggacactctgtgctcgctggggcaggtggcccaccggggcatggagaagagcctctttgtcttcacccaggaggaggtgcaggctccgggctgcaggagagagacatgcagctgggcttcctgcgggctttgcggagctgggccccgggggtgaccagcagtcctatgagtttttccacctcaccctccaggccttctttacagccttcttcctcgtgctggacgacagggtgggcactcaggagctgctcaggttcttccaggagtggatgccccctgcgggggcagcgaccacgtcctgctatcctcccttcctcccgttccagtgcctgcagggcagtggtccggcgcgggaagacctcttcaagaacaaggatcacttccagttcaccaacctcttcctgtgcgggctgttgtccaaagccaaacagaaactcctgcggcatctggtgcccgcggcagccctgaggagaaagcgcaaggccctgtgggcacacctgttttccagcctgcggggctacctgaagagcctgccccgcgttcaggtcgaaagcttcaaccaggtgcaggccatgcccacgttcatctggatgctgcgctgcatctacgagacacagagccagaaggtggggcagctggcggccaggggcatctgcgccaactacctcaagctgacctactgcaacgcctgctcggccgactgcagcgccctctccttcgtcctgcatcacttccccaagcggctggccctagacctagacaacaacaatctcaacgactacggcgtgcgggagctgcagccctgcttcagccgcctcactgttctcagactcagcgtaaaccagatcactgacggtggggtaaaggtgctaagcgaagagctgaccaaatacaaaattgtgacctatttgggtttatacaacaaccagatcaccgatgtcggagccaggtacgtcaccaaaatcctggatgaatgcaaaggcctcacgcatcttaaactgggaaaaaacaaaataacaagtgaaggagggaagtatctcgccctggctgtgaagaacagcaaatcaatctctgaggttgggatgtggggcaatcaagttggggatgaaggagcaaaagccttcgcagaggctctgcggaaccaccccagcttgaccaccctgagtcttgcgtccaacggcatctccacagaaggaggaaagagccttgcgagggccctgcagcagaacacgtctctagaaatactgtggctgacccaaaatgaactcaacgatgaagtggcagagagtttggcagaaatgttgaaagtcaaccagacgttaaagcatttatggcttatccagaatcagatcacagctaaggggactgcccagctggcagatgcgttacagagcaacactggcataacagagatttgcctaaatggaaacctgataaaaccagaggaggccaaagtctatgaagatgagaagcggattatctgtttctgagaggatgctttcctgttcatggggtttttgccctggagcctcagcagcaaatgccactctgggcagtcttttgtgtcagtgtcttaaaggggcctgcgcaggcgggactatcaggagtccactgcctccatgatgcaagccagcttcctgtgcagaaggtctggtcggcaaactccctaagtacccgctacaattctgcagaaaaagaatgtgtcttgcgagctgttgtagttacagtaaatacactgtgaagagactttattgcctattataattatttttatctgaagctagaggaataaagctgtgagcaaacagaggaggccagcctcacctcattccaacacctgccatagggaccaacgggagcgagttggtcaccgctcttttcattgaagagttgaggatgtggcacaaagttggtgccaagcttcttgaataaaacgtgtttgatggattagtattatacctgaaatattttcttccttctcagcactttcccatgtattgatactggtcccacttcacagctggagacaccggagtatgtgcagtgtgggatttgactcctccaaggttttgtggaaagttaatgtcaaggaaaggatgcaccacgggcttttaattttaatcctggagtctcactgtctgctggcaaagatagagaatgccctcagctcttagctggtctaagaatgacgatgccttcaaaatgctgcttccactcagggcttctcctctgctaggctaccctcctctagaaggctgagtaccatgggctacagtgtctggccttgggaagaagtgattctgtccctccaaagaaatagggcatggcttgcccctgtggccctggcatccaaatggctgcttttgtctcccttacctcgtgaagaggggaagtctcttcctgcctcccaagcagctgaagggtgactaaacgggcgccaagactcaggggatcggctgggaactgggccagcagagcatgttggacaccccccaccatggtgggcttgtggtggctgctccatgagggtgggggtgatactactagatcacttgtcctcttgccagctcatttgttaataaaatactgaaaac

SEQ ID NO: 3 - protein, 507 aa, Homo sapiens
MEEQGHSEMEIIPSESHPHIQLLKSNRELLVTHIRNTQCLVDNLLKNDYFSAEDAEIVCACPTQPDKVRKILDLVQSKGEEVSEFFLYLLQQLADAYVDLRPWLLEIGFSPSLLTQSKVVVNTDPVSRYTQQLRHHLGRDSKFVLCYAQKEELLLEEIYMDTIMELVGFSNESLGSLNSLACLLDHTTGILNEQGETIFILGDAGVGKSMLLQRLQSLWATGRLDAGVKFFFHFRCRMFSCFKESDRLCLQDLLFKHYCYPERDPEEVFAFLLRFPHVALFTFDGLDELHSDLDLSRVPDSSCPWEPAHPLVLLANLLSGKLLKGASKLLTARTGIEVPRQFLRKKVLLRGFSPSHLRAYARRMFPERALQDRLLSQLEANPNLCSLCSVPLFCWIIFRCFQHFRAAFEGSPQLPDCTMTLTDVFLLVTEVHLNRMQPSSLVQRNTRSPVETLHAGRDTLCSLGQVAHRGMEKSLFVFTQEEVQAPGCRRETCSWASCGLCGAGPRG

SEQ ID NO: 4 - DNA, 20 bp, Homo sapiens
tagagatgggggtctcacta

SEQ ID NO: 5 - DNA, 20 bp, Homo sapiens
ctcccaaagcactgggatta

SEQ ID NO: 6 - DNA, 20 bp, Homo sapiens
aggtggggtgggctctttct

SEQ ID NO: 7 - DNA, 20 bp, Homo sapiens
acttctcggcggaagatgcg

SEQ ID NO: 8 - DNA, 20 bp, Homo sapiens
tctacatggataccatcatg

SEQ ID NO: 9 - DNA, 20 bp, Homo sapiens
ctgctacagcagctgcagag

SEQ ID NO: 10 - DNA, 20 bp, Homo sapiens
gggtcaaattgttcttccac

SEQ ID NO: 11 - DNA, 20 bp, Homo sapiens
gcgggaccccaaggaggtgt

SEQ ID NO: 12 - DNA, 20 bp, Homo sapiens
acctgagccgtgtgcctgac

SEQ ID NO: 13 - DNA, 20 bp, Homo sapiens
ggccctgcagaaccgcctgc

SEQ ID NO: 14 - DNA, 20 bp, Homo sapiens
cggaacacacacagcccagt

SEQ ID NO: 15 - DNA, 20 bp, Homo sapiens
gtggtccggcacgggaagac

SEQ ID NO: 16 - DNA, 20 bp, Homo sapiens
agcctgccccacgttcaggt

SEQ ID NO: 17 - DNA, 20 bp, Homo sapiens
tagaaggggacggatgtatg

SEQ ID NO: 18 - DNA, 20 bp, Homo sapiens
gctgtgtgtggggggggcgg

SEQ ID NO: 19 - DNA, 21 bp, Homo sapiens
tgtgtgggggggcgggccttg

SEQ ID NO: 20 - DNA, 20 bp, Homo sapiens
gcaactccctcacgaagggg

SEQ ID NO: 21 - DNA, 20 bp, Homo sapiens
atacaagttacgttcttctt

SEQ ID NO: 22 - DNA, 20 bp, Homo sapiens
gtgtgcgagggtccttaaca

SEQ ID NO: 23 - DNA, 20 bp, Homo sapiens
atgtgtcttgtgagctgttg

SEQ ID NO: 24 - DNA, 20 bp, Homo sapiens
atctgaagctggaggaataa

SEQ ID NO: 25 - DNA, 24 bp, Homo sapiens
tattataattaattatttttatct

SEQ ID NO: 26 - DNA, 21 bp, Homo sapiens
catgttggacacccccccacc

SEQ ID NO: 27 - DNA, 20 bp, Homo sapiens
ccaggggtgtaaccagtttg

SEQ ID NO: 28 - DNA, 20 bp, Homo sapiens
tgatccttcataggcctctc

SEQ ID NO: 29 - DNA, 20 bp, Homo sapiens
gacaggaagcagctgtggca

SEQ ID NO: 30 - DNA, 23 bp, Homo sapiens
gatcatcgttcactgcagccttg

SEQ ID NO: 31 - DNA, 22 bp, Homo sapiens
cttgcctggccagataaaggtc

SEQ ID NO: 32 - DNA, 22 bp, Homo sapiens
attgcccttctgctgagaggac

SEQ ID NO: 33 - DNA, 20 bp, Homo sapiens
aggatgcccctcatgccagc

SEQ ID NO: 34 - DNA, 20 bp, Homo sapiens
ctcctcaccctgctgctgtg

SEQ ID NO: 35 - DNA, 22 bp, Homo sapiens
tgagcagggtgagaccatcttc

SEQ ID NO: 36 - DNA, 21 bp, Homo sapiens
tggccctcttcaccttcgatg

SEQ ID NO: 37 - DNA, 19 bp, Homo sapiens
tgcaggaccgcctgctgag

SEQ ID NO: 38 - DNA, 19 bp, Homo sapiens
agtggagaccctccacgcc

SEQ ID NO: 39 - DNA, 21 bp, Homo sapiens
ctgctcaggttcttccaggag

SEQ ID NO: 40 - DNA, 21 bp, Homo sapiens
acacctgttttccagcctgcg

SEQ ID NO: 41 - DNA, 23 bp, Homo sapiens
ataatgagtgcctgccctgactc

SEQ ID NO: 42 - DNA, 24 bp, Homo sapiens
tgacatcaggagccagaaagtctc

SEQ ID NO: 43 - DNA, 21 bp, Homo sapiens
cacattggagcccctgcatag

SEQ ID NO: 44 - DNA, 22 bp, Homo sapiens
actgggcacaaacatctgcctg

SEQ ID NO: 45 - DNA, 24 bp, Homo sapiens
tcccagctgttctgatgttgaagc

SEQ ID NO: 46 - DNA, 21 bp, Homo sapiens
agctcccgctcctgtgaactc

SEQ ID NO: 47 - DNA, 26 bp, Homo sapiens
agccagatacaaagtcacatggactc

SEQ ID NO: 48 - DNA, 24 bp, Homo sapiens
acgagtgtgtgctatgaacacacc

SEQ ID NO: 49 - DNA, 20 bp, Homo sapiens
tgcgcaggcgggactatcag

SEQ ID NO: 50 - DNA, 22 bp, Homo sapiens
agcctcacctcattccaacacc

SEQ ID NO: 51 - DNA, 24 bp, Homo sapiens
atgtcaaggaaaggatgcaccacg

SEQ ID NO: 52 - DNA, 23 bp, Homo sapiens
tgtctcccttacctcgtgaagag

SEQ ID NO: 53 - DNA, 25 bp, Homo sapiens
actgaaaacactcttacgggttgag

SEQ ID NO: 54 - DNA, 23 bp, Homo sapiens
cactggttgtggggtttctctac

SEQ ID NO: 55 - DNA, 22 bp, Homo sapiens
gtagcaagcggctacttttccc

SEQ ID NO: 56 - DNA, 20 bp, Homo sapiens
tccacacaatgccatgcccg

SEQ ID NO: 57 - DNA, 20 bp, Homo sapiens
tgcttgcactggtgcctgcg

SEQ ID NO: 58 - DNA, 21 bp, Homo sapiens
tagcagcatggacttgcccac

SEQ ID NO: 59 - DNA, 20 bp, Homo sapiens
agtccgagtgcagctcgtcc

SEQ ID NO: 60 - DNA, 20 bp, Homo sapiens
gcacaggctgcagaggttgg

SEQ ID NO: 61 - DNA, 23 bp, Homo sapiens
acaaagaggctcttctccatgcc

SEQ ID NO: 62 - DNA, 19 bp, Homo sapiens
agcaggacgtggtcgctgc

SEQ ID NO: 63 - DNA, 20 bp, Homo sapiens
aacgtgggcatggcctgcac

SEQ ID NO: 64 - DNA, 20 bp, Homo sapiens
attcccgatgccctccgagc

SEQ ID NO: 65 - DNA, 24 bp, Homo sapiens
gaatgtgaattccctgcagctctg

SEQ ID NO: 66 - DNA, 24 bp, Homo sapiens
ggacaagtggaaggctttaggatg

SEQ ID NO: 67 - DNA, 24 bp, Homo sapiens
agtggtccttctggtgtactgatg

SEQ ID NO: 68 - DNA, 25 bp, Homo sapiens
tattaggagcacgaatcacccttcc

SEQ ID NO: 69 - DNA, 24 bp, Homo sapiens
tggacgacaaagcaagaccctatc

SEQ ID NO: 70 - DNA, 25 bp, Homo sapiens
tagaattaggtggtgatggttgcac

SEQ ID NO: 71 - DNA, 25 bp, Homo sapiens
acagtcagtggtggtgagtaaacag

SEQ ID NO: 72 - DNA, 23 bp, Homo sapiens
acaggaagctggcttgcatcatg

SEQ ID NO: 73 - DNA, 21 bp, Homo sapiens
agagcggtgaccaactcgctc

SEQ ID NO: 74 - DNA, 21 bp, Homo sapiens
tgccagcagacagtgagactc

SEQ ID NO: 75 - DNA, 20 bp, Homo sapiens
tcagctgcttgggaggcagg

SEQ ID NO: 76 - DNA, 23 bp, Homo sapiens
tgagtgcttaccaagccaagtcc

SEQ ID NO: 77 - DNA, 26 bp, Homo sapiens
acaagttatttcctctgagaagccac

SEQ ID NO: 78 - DNA, 10 bp, Homo sapiens
tgtcattgat

SEQ ID NO: 79 - DNA, 10 bp, Homo sapiens
tcacctccag

SEQ ID NO: 80 - DNA, 10 bp, Homo sapiens
ctccctccag

SEQ ID NO: 81 - DNA, 10 bp, Homo sapiens
tttatttcag

SEQ ID NO: 82 - DNA, 10 bp, Homo sapiens
tctcttgaag

SEQ ID NO: 83 - DNA, 10 bp, Homo sapiens
ctctctgcag

SEQ ID NO: 84 - DNA, 10 bp, Homo sapiens
cctccctcag

SEQ ID NO: 85 - DNA, 10 bp, Homo sapiens
tccttcctag

SEQ ID NO: 86 - DNA, 10 bp, Homo sapiens
ttttccttag

SEQ ID NO: 87 - DNA, 10 bp, Homo sapiens
cttctttcag

SEQ ID NO: 88 - DNA, 10 bp, Homo sapiens
ttttcaacag

SEQ ID NO: 89 - DNA, 10 bp, Homo sapiens
ctcttcaggg

SEQ ID NO: 90 - DNA, 10 bp, Homo sapiens
gtccgcaaaa

SEQ ID NO: 91 - DNA, 10 bp, Homo sapiens
tgagcaggta

SEQ ID NO: 92 - DNA, 10 bp, Homo sapiens
actcagcgta

SEQ ID NO: 93 - DNA, 10 bp, Homo sapiens
tttatacaac

SEQ ID NO: 94 - DNA, 10 bp, Homo sapiens
actgggaaaa

SEQ ID NO: 95 - DNA, 10 bp, Homo sapiens
gatgtggggc

SEQ ID NO: 96 - DNA, 10 bp, Homo sapiens
tcttgcgtcc

SEQ ID NO: 97 - DNA, 10 bp, Homo sapiens
gctgacccaa

SEQ ID NO: 98 - DNA, 10 bp, Homo sapiens
gcttatccag

SEQ ID NO: 99 - DNA, 10 bp, Homo sapiens
cctaaatgga

SEQ ID NO: 100 - DNA, 10 bp, Homo sapiens
gcctgacaag

SEQ ID NO: 101 - DNA, 10 bp, Homo sapiens
actgacccag

SEQ ID NO: 102 - DNA, 10 bp, Homo sapiens
ctgttctcag

SEQ ID NO: 103 - DNA, 10 bp, Homo sapiens
cctatttggg

SEQ ID NO: 104 - DNA, 10 bp, Homo sapiens
cgcatcttaa

SEQ ID NO: 105 - DNA, 10 bp, Homo sapiens
ctgaggttgg

SEQ ID NO: 106 - DNA, 10 bp, Homo sapiens
ccaccctgag

SEQ ID NO: 107 - DNA, 10 bp, Homo sapiens
aaatactgtg

SEQ ID NO: 108 - DNA, 10 bp, Homo sapiens
agcatttatg

SEQ ID NO: 109 - DNA, 10 bp, Homo sapiens
cagagatttg

SEQ ID NO: 110 - DNA, 10 bp, Homo sapiens
cttgtcctct

SEQ ID NO: 111 - DNA, 10 bp, Homo sapiens
gtgccccggg

SEQ ID NO: 112 - DNA, 10 bp, Homo sapiens
gtaggagtca

SEQ ID NO: 113 - DNA, 10 bp, Homo sapiens
gtgaggctgc

SEQ ID NO: 114 - DNA, 10 bp, Homo sapiens
gtatgtcttt

SEQ ID NO: 115 - DNA, 10 bp, Homo sapiens
gtaagtgggg

SEQ ID NO: 116 - DNA, 10 bp, Homo sapiens
gtgagtagaa

SEQ ID NO: 117 - DNA, 10 bp, Homo sapiens
gtaactgtgg

SEQ ID NO: 118 - DNA, 10 bp, Homo sapiens
gtaatagctc

SEQ ID NO: 119 - DNA, 10 bp, Homo sapiens
gtaactcaga

SEQ ID NO: 120 - DNA, 10 bp, Homo sapiens
gtaagatccc

SEQ ID NO: 121 - DNA, 10 bp, Homo sapiens
tgccagctca

(19) United States
(12) Patent Application Publication
(10) Pub. No.: US 2003/0219810 A1
(43) Pub. Date: Nov. 27, 2003

(54) Polymorphisms in the human CARD4 gene

(76) Inventors: Glenn Barnes, Haverhill, MA (US); John Bertin, Toronto (CA)

Correspondence Address: Jean M. Silveri, Millennium Pharmaceuticals, Inc., 75 Sidney Street, Cambridge, MA, 02139, US

(21) Appl. No.: 10/401,194

(22) Filed: Mar. 27, 2003

Related U.S. Application Data
(60) Provisional application No. 60/368,184, filed on Mar. 27, 2002.

(51) Int. Cl. C12Q 1/68; C07H 21/04

(52) U.S. Cl. 435/6; 536/23.2

(57) ABSTRACT

The present invention is based at least in part on the discovery of polymorphisms within the CARD4 gene. Accordingly, the invention provides nucleic acid molecules having a nucleotide sequence of an allelic variant of a CARD4 gene. The invention also provides methods for identifying specific alleles of polymorphic regions of a CARD4 gene, methods for determining whether a patient has a more or less severe phenotype of an inflammatory or allergic or apoptotic disease or disorder, methods for determining whether a patient will be more or less responsive to a given treatment for such a disorder, forensic methods based on detection of polymorphisms within the CARD4 gene, and kits for performing such methods.

### RELATED PATENTS

This application claims priority from U.S. Provisional Application Serial No. 60/368,184, filed Mar. 27, 2002, hereby incorporated by reference.

### BACKGROUND

CARD4 (NOD1), a member of the CED4/Apaf-1 family, is an important signaling protein involved in apoptosis and inflammation. CARD4 activates caspase-9 induced apoptosis (Bertin et al. (1999) J. Biol. Chem. 19:12955). In addition, CARD4 interacts with the serine-threonine kinase RICK and activates NF-κB signaling using a TRAF-6/NIK signaling pathway Inohara et al. (1999) J. Biol. Chem. 274:14560-67). CARD4 bears some resemblance to plant disease resistance protein (Bertin et al., supra). Through self-association and induced proximity of binding partners, CARD4 is thought to activate diverse signaling pathways that eliminate cells through programmed cell death and host defense mechanisms directed against pathogens. For example, CARD4 plays a role in a cytoplasmic detection system for LPS (Girardin et al. (2001) EMBO Rep. 2:736).

### SUMMARY

The present invention relates to polymorphisms in the CARD4 gene and is based, in part, on the discovery of 26 CARD4 polymorphisms (Table 1). CARD4 (also referred to as NOD 1) is a 953 amino acid human protein having a CARD motif (amino acids 15-114), a nucleotide binding domain (amino acids 198-397), and ten leucine rich repeats (amino acids 674-950). CARD4 is described in detail in U.S. Ser. Nos. 09/099,041 (filed Jun. 17, 1998); 09/207,359 (filed Dec. 8, 1998); 09/245,281 (filed Feb. 5, 1999); 09/748,537 (filed Dec. 26, 2000); 09/728,721 (filed Dec. 1, 2000); and 09/841,879 (filed Apr. 24, 2001), hereby incorporated by reference.

CARD4 is involved in apoptosis and inflammation. It may play a role in chronic obstructive pulmonary disease, rheumatoid arthritis, inflammatory bowel disease, and psoriasis. Recently, a genome-wide scan in a founder population revealed a susceptibility locus for asthma-related traits (i.e., asthma, high levels of IgE, and asthma combined with high levels of IgE) in a 20 cM region of chromosome 7p14-p15 (Laitinen et al. (2001) 28:87-91). The CARD4 gene maps to the same region of chromosome 7. It is possible that CARD4 plays a role in the development of asthma.

The invention provides a method for identifying an individual having a variant CARD4 gene, the method including determining the presence or absence of a CARD4 allelic variant comprising a sequence selected from the group consisting of those set forth in SEQ ID NOs: 4-29 or the complement of the sequence in a nucleic acid sample from the individual or the identity of the nucleotide present at the polymorphic site on either strand. The presence of a CARD4 allelic variant in the sample can indicate that the individual has a particular response to a drug used for treatment of an inflammatory or allergic or apoptotic disorder or a particular susceptibility to developing an inflammatory or allergic or apoptotic disorder. In one embodiment, the CARD4 allelic variant comprises the sequences of those set forth in SEQ ID NOs4-29, or the complement of each of those sequences. In another embodiment, the CARD4 allelic variant comprises the sequences of those set forth in two or more of SEQ ID NOs: 4-29, or the complement of each of those sequences. In a preferred embodiment, the individual is suffering from an inflammatory or allergic or apoptotic disorder, e.g., asthma.

The invention also provides isolated nucleic acids comprising one or more of the novel CARD4 polymorphisms of the invention. The nucleic acid molecules of the invention include specific CARD4 allelic variants which differ from the reference CARD4 sequences set forth in SEQ ID NO: 1 (GenBank® Reference No. GI 415649; CARD4 genomic DNA reference sequence), and SEQ ID NO: 2 (GenBank® Reference No. GI 11419372; CARD4 cDNA reference sequence). The amino acid encoded by SEQ ID NO: 2 is shown in SEQ ID NO: 3. The preferred nucleic acid molecules of the invention comprise newly identified CARD4 allelic variants or portions thereof having any of the novel polymorphisms shown in Table 1 (i.e., those comprising the sequence of any of those set forth in SEQ ID NOs: 4-29 or the complement thereof), polymorphisms in linkage disequilibrium with the polymorphisms shown in Table 1, and combinations thereof.

The nucleic acid molecules of the invention can be double- or single-stranded. Accordingly, in one embodiment of the invention, a complement of the nucleotide sequence is provided wherein the polymorphism has been identified. For example, where there has been a single nucleotide change from a guanine to an adenine in a single strand, the complement of that strand will contain a change from cytosine to thymine at the corresponding nucleotide residue. Nucleic acids of the invention can function as probes or primers, e.g., in methods for determining the allelic identity of a CARD4 polymorphic region in a nucleic acid of interest. The invention further provides vectors comprising the nucleic acid molecules of the present invention; host cells transfected with such vectors whether prokaryotic or eukaryotic; and transgenic non-human animals which contain a heterologous form of a functional or non-functional CARD4 allele described herein. Such a transgenic animal can serve as an animal model for studying the effect of specific CARD4 allelic variations, including mutations, as well as for use in drug screening and/or recombinant protein production.

The invention further provides methods for determining the molecular structure of at least a portion of a CARD4 gene. In a preferred embodiment, the method comprises contacting a sample nucleic acid comprising a CARD4 gene sequence with a probe or primer having a sequence which is complementary to a CARD4 gene sequence, carrying out a reaction that would amplify and/or detect differences in a region of interest within the CARD4 gene sequence, and comparing the result of each reaction with that of a reaction with a control (known) CARD4 gene (e.g., a CARD4 gene from a human not afflicted with an inflammatory condition, e.g., asthma, or another disease associated with an aberrant CARD4 activity) so as to determine the molecular structure of the CARD4 gene sequence in the sample nucleic acid. The method of the invention can be used for example in determining the molecular structure of at least a portion of an exon, an intron, a 5′ upstream regulatory element, or the 3′ untranslated region. In a preferred embodiment, the method comprises determining the identity of at least one nucleotide. In another preferred embodiment, the method comprises determining the nucleotide at one or more of residues 68444, 68365, 68258, 67932, 64100, 63942, 63893, 63787, 63692, 63469, 63243, 62863, 62675, 57293, 57262, 57256, 40687, 40639, 36926, 36587, 36513, 36529, 35808, 35552, 35534, and 35502 of the reference sequence GI 4156149 (SEQ ID NO: 1).

In another preferred embodiment, the method comprises determining the nucleotide content of at least a portion of a CARD4 gene, such as by sequence analysis. In yet another embodiment, determining the molecular structure of at least a portion of a CARD4 gene is carried out by single-stranded conformation polymorphism (SSCP). In yet another embodiment, the method is an oligonucleotide ligation assay (OLA). Other methods within the scope of the invention for determining the molecular structure of at least a portion of a CARD4 gene include hybridization of allele-specific oligonucleotides, sequence specific amplification, primer specific extension, and denaturing high performance liquid chromatography (DHPLC). In at least some of the methods of the invention, the probe or primer is allele specific. Preferred probes or primers are single stranded nucleic acids, which optionally are labeled.

The invention further provides forensic methods based on detection of polymorphisms in the CARD4 gene.

The invention also provides probes and primers comprising oligonucleotides which hybridizes to at least 6, 10, 15, or 20 consecutive nucleotides of any of the sequences set forth in SEQ ID NOs: 4-29, or to the complement of any of such sequences, or naturally occurring mutants or variants thereof. In preferred embodiments, the probe/primer further includes a label attached thereto, which is capable of being detected.

In another embodiment, the invention provides a kit for amplifying and/or for determining the molecular structure of at least a portion of a CARD4 gene, comprising a probe or primer capable of hybridizing to a CARD4 gene and instructions for use. In one embodiment, the probe or primer is capable of hybridizing to a CARD4 intron. In another embodiment, the probe or primer is capable of hybridizing to a CARD4 allelic variant, preferably a variant corresponding to those in Table 1.

In another embodiment, the polymorphic region is located in the 5′ upstream regulatory element, an exon, or an intron. In a preferred embodiment, determining the molecular structure of a region of a CARD4 gene comprises determining the identity of the allelic variant of the polymorphic region. Determining the molecular structure of at least a portion of a CARD4 gene can comprise determining the identity of at least one nucleotide or determining the nucleotide composition, e.g., the nucleotide sequence.

A method or kit of the invention can be used, e.g., for determining whether a patient will or will not be responsive to effective treatment of a disease associated with a specific CARD4 allelic variant with a CARD4 modulator or some other therapeutic agent. In a preferred embodiment, the invention provides a method and a kit for determining whether a patient will or will not be responsive to treatment of a inflammatory or allergic disease or condition. The method and kit of the invention can also be used in selecting the appropriate drug to administer to a patient to treat such a disease or condition. The method and kit of the invention can also be used to determine and individual's susceptibility to an apoptotic, inflammatory, or allergic disease or disorder.

In another aspect, the invention provides a method and a kit for determining whether an inflammatory or allergic or apoptotic disease patient has a more moderate or more severe disease phenotype associated with a specific CARD4 allelic variant of a polymorphic region. In one embodiment, the disease or disorder is characterized by an abnormal CARD4 activity, e.g., aberrant CARD4 expression. In another embodiment, the disease or disorder is characterized by an abnormal CARD4 activity.

Other features and advantages of the invention will be apparent from the following detailed description and claims.

### DETAILED DESCRIPTION

The present invention is based, in part, on the identification of polymorphisms in the CARD4 gene.

Pharmacogenetic studies have shown that the genetic background of an individual plays a role in determining the response of the individual to a specific drug. Thus, determining the allelic variants of CARD4 polymorphic regions of an individual can be useful in predicting how an individual will respond to a specific drug, e.g., a drug for treating a disease or disorder associated with aberrant CARD4 activity and/or an inflammatory or allergic or apoptotic disorder. For example, specific CARD4 polymorphisms comprising one or more polymorphisms of the present invention may result in increased or decreased production or expression of the CARD4 polypeptide. Accordingly, the action of a drug necessitating interaction with a CARD4 protein will be different in individuals carrying a CARD4 allele containing a polymorphism. Furthermore, identification of polymorphisms in the CARD4 gene which indicate responsiveness to a therapy are beneficial in the treatment of diseases because they can serve as markers by which the most appropriate treatment can be identified.

In addition, the genetic background of an individual can be used to predict the individuals susceptibility to a disease or disorder. Thus, determining the allelic variants of CARD4 polymorphic regions of an individual can be useful in predicting the individual's susceptibility to an inflammatory, apoptotic or allergic disease or disorder, e.g., in chronic obstructive pulmonary disease, rheumatoid arthritis, inflammatory bowel disease, psoriasis or asthma.

There are multiple alleles of the CARD4 gene. The reference CARD4 gene sequence designated herein is presumed to be the wild-type CARD4 gene sequence and comprises nucleotide sequences that have been deposited in GenBank® and assigned the Identification Numbers GI 4156149 and GI 11419372 (corresponding to SEQ ID NO: 1 and SEQ ID NO: 2, respectively). The present invention relates to variant alleles of the CARD4 gene that differ from the reference CARD4 gene sequence by at least one of the polymorphisms identified in Table 1, and those in linkage equilibrium therewith. The present invention thus relates to nucleic acids comprising such variant CARD4 alleles.

The invention further relates to nucleic acids comprising portions of such variant CARD4 alleles that contain any of the novel CARD4 polymorphisms identified in Table 1 and are at least 5 nucleotides or basepairs in length. Portions can be, for example, 5-10, 5-15, 10-20, 2-25, 10-30, 10-50 or 10-100 nucleotides or basepairs long. For example, a portion of a variant allele which is 21 nucleotides or basepairs in length includes a CARD4 polymorphism (i.e., a nucleotide which differs from the corresponding nucleotide in the reference CARD4 sequence) and twenty additional nucleotides or basepairs which flank the polymorphism in the variant allele. These additional nucleotides and basepairs can be on one or both sides of the polymorphism. Polymorphisms of the invention are defined in Table 1 with respect to specific reference CARD4 sequences identified in Table I (GenBank® GI 4156149 (SEQ ID NO: 1) or GenBank® GI 11419372(SEQ ID NO: 2)).

The CARD4 polymorphisms of the present invention have been identified in the human CARD4 gene by analyzing the DNA of human populations. In particular, DNA samples from 96 individuals were obtained and used for polymorphism discovery. These 96 DNA samples included samples from a population of 24 North American Caucasian individuals, 24 African American individuals, and 24 Asian Chinese individuals from throughout the Anhui province in East Central China, and 24 Asian Chinese asthmatics.

The allelic variants of the present invention were identified by performing denaturing high performance liquid chromatography (DHPLC) analysis, the polymerase chain reaction (PCR), and/or single stranded conformation polymorphism (SSCP) analysis of genomic DNA from independent individuals as described in Example 1, using PCR primers complementary to intronic sequences surrounding each of the exons, 3′ UTR, and 5′ upstream regulatory element sequences of the CARD4 gene. The nucleotide sequence of these PCR primers (having SEQ ID NOs.: 29-76) is shown in Table 3 (see the Examples).

The presence of 26 novel polymorphisms in the human CARD4 gene were identified in the populations studied. All but three of the polymorphisms were characterized as single nucleotide polymorphisms (SNPs). The three remaining polymorphisms comprise insertions of one or more nucleotides from the reference CARD4 sequence. These variants are referred to herein as a “insertion variants.”

Table 1 contains a “Polymorphism ID No.” in column 1, which is used herein to identify each individual CARD4 polymorphism. The nucleotide sequence flanking each polymorphism is provided in column 3, in which the polymorphic residue(s), having the wild-type or reference nucleotide, is indicated in lower-case letters. There are 10 nucleotides flanking the polymorphic nucleotide residue (i.e., 10 nucleotides 5′ of the polymorphism and 9 nucleotides 3′ of the polymorphism). Column 2 also indicates the sequence listing identifier number (SEQ ID NO.) of the sequence shown in column 3 but with a variant nucleotide at the residue(s) shown in lower-case letter(s) or with a deletion of the sequences contained within the parenthesis. For example, SEQ ID NO: 4 contains an adenine (“g”) at the location indicated by the lower-case letter “t” in the corresponding sequence in column 3. Therefore, SEQ ID NO: 4 is identical to the corresponding sequence in column 2, except that the “t” (thymidine) residue is replaced by an “g” (guanine residue). Column 4 of Table 1 indicates the exon location for each polymorphisms located in an exon. Columns 5-7 of Table 1 indicate the reference codon (column 5), variant codon (column 6), and amino acid (column 7) for each silent polymorphism in the CARD4 coding sequence. Columns 8-10 of Table 1 indicate the reference codon (column 8), variant codon (column 9), and amino acid change (column 10) for each non-silent CARD4 polymorphism in the CARD4 coding sequence. Column 11 of Table 1 provides the nucleotide change (or insertion) and location (promoter, 3′ UTR, intron) for each non-coding region polymorphism. Column 12 of Table 1 provides the nucleotide position within either GenBank® GI 4156149 (SEQ ID NO: 1) or GenBank® GI11419372 (SEQ ID NO: 2) (or both) for each polymorphism. As explained in greater detail below, the sequence shown in column 3 of Table 1 is the reverse complement of the sequence in GenBank® GI 4156149 (SEQ ID NO: 1). Nevertheless, the nucleotide position of the polymorphism listed in column 11 for GenBank® GI 4156149 (SEQ ID NO: 1) is accurate. Columns 13-16 of Table 1 provide the allele frequency for each polymorphism in the African American (AAC; column 13), North American Caucasian (1MR; column 14), Asian Chinese (ANQ; column 15), and Asian Chinese asthmatic (column 16) population studied.

Each polymorphism is identified based on a change in the nucleotide sequence from a “reference sequence.” As used herein, the reference sequence is the nucleotide sequence of SEQ ID NO: 1 which corresponds to GenBank® GI 4156149 or the nucleotide sequence of SEQ ID NO: 2 which corresponds to GenBank® GI 11419372. To identify the location of each polymorphism in Table 1, a specific nucleotide residue in a reference sequence is listed for each polymorphism (column 12), where nucleotide residue number 1 is the first (i.e., most 5′) nucleotide in GenBank® GI 4156149 (corresponding to SEQ ID NO: 1), or the first nucleotide in GenBank® GI 11419372 (corresponding to SEQ ID NO: 2).

The nucleic acid molecules of the invention can be double- or single-stranded. Accordingly, the invention further provides for the nucleic acid strands comprising sequences complementary SEQ ID Nos: 4-29.

The invention further provides allele-specific oligonucleotides that hybridize to a gene comprising a polymorphism of the invention. Such oligonucleotides will hybridize to one polymorphic form of the nucleic acid molecules described herein but not to the other polymorphic form(s) of the sequence. Thus such oligonucleotides can be used to determine the presence or absence of particular alleles of the polymorphic sequences described herein. These oligonucleotides can be probes or primers.

Not only does the present invention provide for polymorphisms in linkage disequilibrium with the polymorphisms of Table 1, it also provides methods for revealing the existence of yet other polymorphisms in the human CARD4 gene. For example, the polymorphism studies described herein can also be applied to populations in which other inflammatory or allergic or apoptotic diseases or disorders are prevalent.

Other aspects of the invention are described below or will be apparent to one of skill in the art in light of the present disclosure.

Definitions

For convenience, the meaning of certain terms and phrases employed in the specification, examples, and appended claims are provided below.

The term “inflammatory disease or allergic disease or disorder” as used herein refers to any disease or disorder characterized by an aberrant inflammatory response. Examples of inflammatory or allergic diseases or disorders include, but are not limited to, asthma, bronchitis, sinusitis, ulcerative colitis, nephritis, amyloidosis, rheumatoid arthritis, sarcoidosis, scleroderma, lupus, non-allergic rhinitis, polymyositis, Reiter's syndrome, psoriasis, pelvic inflammatory disease, orbital inflammatory disease, thrombotic disease, and inappropriate allergic responses to environmental stimuli such as poison ivy, pollen, insect stings and certain foods, including atopic dermatitis and contact dermatitis, multiple sclerosis and Crohn's disease, chronic obstructive pulmonary disease, inflammatory bowel disease, and psoriasis.

The term “allele,” which is used interchangeably herein with “allelic variant” and “variant allele”, refers to alternative forms of a gene or portions thereof. Alleles occupy the same locus or position on homologous chromosomes. When a patient has two identical alleles of a gene, the patient is said to be homozygous for the gene or allele. When a patient has two different alleles of a gene, the patient is said to be heterozygous for the gene. Alleles of a specific gene, including CARD4, can differ from each other in a single nucleotide, or several nucleotides, and can include substitutions, deletions, and insertions of nucleotides. An allele of a gene can also be a form of a gene containing one or more mutations.

The term “allelic variant of a CARD4 gene” or “CARD4 allelic variant” refers to an alternative form of the CARD4 gene having one of several possible nucleotide sequences found in same position within the gene in the population. The predominate alleles in the population are referred to as “wild-type” alleles.

“Biological activity” or “bioactivity” or “activity” or “biological function”, which are used interchangeably, for the purposes herein when applied to CARD4, means an effector or antigenic function that is directly or indirectly performed by a CARD4 polypeptide (whether in its native or denatured conformation), or by a fragment thereof. Biological activities include the ability to interact with a procaspase containing a CARD or death effector domain (DED), the ability to interact with RICK, the ability to interact with CLARP (CFLAR), the ability to stimulate caspase-9 mediated apoptosis, the ability to interact with caspse-9, and other biological activities, whether presently known or inherent. A CARD4 bioactivity can be modulated by directly affecting a CARD4 protein effected by, for example, changing the level of effector or substrate level. Alternatively, a CARD4 bioactivity can be modulated by modulating the level of a CARD4 protein, such as by modulating expression of a CARD4 gene. Antigenic functions include possession of an epitope or antigenic site that is capable of cross-reacting with antibodies that bind a native or denatured CARD4 polypeptide or fragment thereof.

Biologically active CARD4 polypeptides include polypeptides having both an effector and antigenic function, or only one of such functions. CARD4 polypeptides include antagonist polypeptides and native CARD4 polypeptides, provided that such antagonists include an epitope of a native CARD4 polypeptide. An effector function of CARD4 polypeptide can be the ability to bind to caspase-9.

As used herein the term “bioactive fragment of a CARD4 protein” refers to a fragment of a full-length CARD4 protein, wherein the fragment specifically mimics or antagonizes the activity of a wild-type CARD4 protein. The bioactive fragment preferably is a fragment capable of binding to a second molecule, such as a ligand.

The term “an aberrant activity” or “abnormal activity”, as applied to an activity of a protein such as CARD4, refers to an activity which differs from the activity of the wild-type (i.e., normal) protein or which differs from the activity of the protein in a healthy subject, e.g., a subject not afflicted with a disease associated with a CARD4 allelic variant. An activity of a protein can be aberrant because it is stronger than the activity of its wild-type counterpart. Alternatively, an activity of a protein can be aberrant because it is weaker or absent relative to the activity of its wild-type counterpart. An aberrant activity can also be a change in reactivity. For example an aberrant protein can interact with a different protein or ligand relative to its wild-type counterpart. A cell can also have aberrant CARD4 activity due to overexpression or underexpression of the CARD4 gene. Aberrant CARD4 activity can result from a mutation in the gene, which results, e.g., in lower or higher binding affinity of the CARD of a CARD-containing protein to the CARD4 protein encoded by the mutated gene. Aberrant CARD4 activity can also result from a lower or higher level of CARD4 expression in cells, which can result, e.g., from a mutation in the 5′ flanking region of the CARD4 gene or any other regulatory element of the CARD4 gene, such as a regulatory element located in an intron. Accordingly, aberrant CARD4 activity can result from an abnormal CARD4 5′ upstream regulatory element activity.

The terms “abnormal CARD4 5′ upstream regulatory element activity”, “aberrant CARD4 5′ upstream regulatory element activity”, “abnormal CARD4 promoter activity”, “aberrant CARD4 promoter activity”, “abnormal CARD4 transcriptional activity” and “aberrant CARD4 transcriptional activity”, which are used interchangeably herein, refer to the transcriptional activity of a CARD4 5′ upstream regulatory element which differs from the transcriptional activity of the corresponding 5′ upstream regulatory element in the wild-type CARD4 allele. Abnormal CARD4 activity can result from a higher or lower transcriptional activity as compared to transcriptional activity of a wild-type CARD4 allele. Aberrant CARD4 5′ upstream regulatory element activity can result, for example, from the presence of a genetic lesion in a regulatory element, such as in a 5′ upstream regulatory element. An “aberrant CARD4 5′ upstream regulatory element activity” is also intended to refer to the transcriptional activity of a CARD4 5′ upstream regulatory element which is functional (capable of inducing transcription of a gene to which it is operably linked) in tissues or cells in which the normal or wild-type CARD4 5′ upstream regulatory element is not functional or which is non functional in tissues or cells in which the normal or wild-type CARD4 5′ upstream regulatory element is functional. Thus, a tissue distribution of CARD4 in a patient which differs from the tissue distribution of CARD4 in a normal (e.g., healthy) individual, can be the result of abnormal transcriptional activity from the CARD4 5′ upstream regulatory element. Such abnormal transcriptional activity can result from e.g., one or more mutations in a regulatory element, such as in a 5′ upstream regulatory element thereof. Abnormal transcriptional activity can also result from a mutation in a transcription factor involved in the control of CARD4 gene expression.

“Cells,” “host cells” or “recombinant host cells” are terms used interchangeably herein. It is understood that such terms refer not only to the particular patient cell but to the progeny or derivatives of such a cell. Because certain modifications may occur in succeeding generations due to either mutation or environmental influences, such progeny may not, in fact, be identical to the parent cell, but are still included within the scope of the term as used herein.

As used herein, the term “gene” or “recombinant gene” refers to a nucleic acid molecule comprising an open reading frame and including at least one exon and (optionally) an intron sequence. The term “intron” refers to a DNA sequence present in a given gene which is spliced out during mRNA maturation.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present invention.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical positions/total number of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score =100, wordlength =12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The term “a homolog of a nucleic acid” refers to a nucleic acid having a nucleotide sequence having a certain degree of homology with the nucleotide sequence of the nucleic acid or complement thereof. For example, a homolog of a double stranded nucleic acid having SEQ ID NO: N is intended to include nucleic acids having a nucleotide sequence which has a certain degree of homology with SEQ ID NO: N or with the complement thereof. Preferred homologs of nucleic acids are capable of hybridizing to the nucleic acid or complement thereof. The term “hybridization probe” or “primer” as used herein is intended to include oligonucleotides which hybridize bind in a base-specific manner to a complementary strand of a target nucleic acid. Such probes include peptide nucleic acids, and described in Nielsen et al., (1991) Science 254:1497-1500. Probes and primers can be any length suitable for specific hybridization to the target nucleic acid sequence. The most appropriate length of the probe and primer may vary depending on the hybridization method in which it is being used; for example, particular lengths may be more appropriate for use in microfabricated arrays, while other lengths may be more suitable for use in classical hybridization methods. Such optimizations are known to the skilled artisan. Suitable probes and primers can range from about 5 nucleotides to about 30 nucleotides in length. For example, probes and primers can be 5, 6, 8, 10, 12, 14, 16, 18, 20, 22, 24, 25, 26, 28 or 30 nucleotides in length. The probe or primer of the invention comprises a sequence that flanks and/or preferably overlaps, at least one polymorphic site occupied by any of the possible variant nucleotides. The nucleotide sequence of an overlapping probe or primer can correspond to the coding sequence of the allele or to the complement of the coding sequence of the allele.

The term “interact” as used herein is meant to include detectable interactions between molecules, such as can be detected using, for example, a binding or hybridization assay. The term interact is also meant to include “binding” interactions between molecules. Interactions may be, for example, protein-protein, protein-nucleic acid, protein-small molecule or small molecule-nucleic acid in nature.

The term “intronic sequence” or “intronic nucleotide sequence” refers to the nucleotide sequence of an intron or portion thereof.

The term “isolated” as used herein with respect to nucleic acids, such as DNA or RNA, refers to molecules separated from other DNAs or RNAs, respectively, that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

The term “linkage” describes the tendency of genes, alleles, loci or genetic markers to be inherited together as a result of their location on the same chromosome. It can be measured by percent recombination between the two genes, alleles, loci, or genetic markers. The term “linkage disequilibrium” refers to a greater than random association between specific alleles at two marker loci within a particular population. In general, linkage disequilibrium decreases with an increase in physical distance. If linkage disequilibrium exists between two markers within one gene, then the genotypic information at one marker can be used to make probabilistic predictions about the genotype of the second marker.

The term “locus” refers to a specific position in a chromosome. For example, a locus of a CARD4 gene refers to the chromosomal position of the CARD4 gene.

The term “modulation” as used herein refers to both upregulation, (i.e., activation or stimulation), for example by agonizing; and downregulation (i.e. inhibition or suppression), for example by antagonizing of a bioactivity (e.g. expression of a gene).

The term “molecular structure” of a gene or a portion thereof refers to the structure as defined by the nucleotide content (including deletions, substitutions, additions of one or more nucleotides), the nucleotide sequence, the state of methylation, and/or any other modification of the gene or portion thereof.

The term “mutated gene” refers to an allelic form of a gene that differs from the predominant form in a population. A mutated gene is capable of altering the phenotype of a patient having the mutated gene relative to a patient having the predominant form of the gene. If a patient must be homozygous for this mutation to have an altered phenotype, the mutation is said to be recessive. If one copy of the mutated gene is sufficient to alter the phenotype of the patient, the mutation is said to be dominant. If a patient has one copy of the mutated gene and has a phenotype that is intermediate between that of a homozygous and that of a heterozygous (for that gene) patient, the mutation is said to be co-dominant.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenosine”, “cytidine”, “guanosine”, and thymidine” are used. It is understood that if the nucleic acid is RNA, a nucleotide having a uracil base is uridine.

The term “nucleotide sequence complementary to the nucleotide sequence set forth in SEQ ID NO: N” refers to the nucleotide sequence of the complementary strand of a nucleic acid strand having SEQ ID NO: N. The term “complementary strand” is used herein interchangeably with the term “complement”. The complement of a nucleic acid strand can be the complement of a coding strand or the complement of a non-coding strand. When referring to double stranded nucleic acids, the complement of a nucleic acid having SEQ ID NO: N refers to the complementary strand of the strand having SEQ ID NO: N or to any nucleic acid having the nucleotide sequence of the complementary strand of SEQ ID NO: N. When referring to a single stranded nucleic acid having the nucleotide sequence SEQ ID NO: N, the complement of this nucleic acid is a nucleic acid having a nucleotide sequence which is complementary to that of SEQ ID NO: N. The nucleotide sequences and complementary sequences thereof are always given in the 5′ to 3′ direction. The term “complement” and “reverse complement” are used interchangeably herein.

A “non-human animal” of the invention can include mammals such as rodents, non-human primates, sheep, goats, horses, dogs, cows, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding and identifying agents which can affect, for example, embryogenesis and tissue formation. The term “chimeric animal” is used herein to refer to animals in which an exogenous sequence is found, or in which an exogenous sequence is expressed in some but not all cells of the animal. The term “tissue-specific chimeric animal” indicates that an exogenous sequence is present and/or expressed or disrupted in some tissues, but not others.

The term “oligonucleotide” is intended to include and single- or double stranded DNA or RNA. Oligonucleotides can be naturally occurring or synthetic, but are typically prepared by synthetic means. Preferred oligonucleotides of the invention include segments of CARD4 gene sequence or their complements, which include and/or flank any one of the polymorphic sites shown in Table 1. The segments can be between 5 and 250 bases, and, in specific embodiments, are between 5-10, 5-20, 10-20, 10-50, 20-50 or 10-100 bases. For example, the segments can be 21 bases. The polymorphic site can occur within any position of the segment or a region next to the segment. The segments can be from any of the allelic forms of CARD4 gene sequence shown in Table 1.

The term “operably-linked” is intended to mean that the 5′ upstream regulatory element is associated with a nucleic acid in such a manner as to facilitate transcription of the nucleic acid from the 5′ upstream regulatory element.

The term “polymorphism” refers to the coexistence of more than one form of a gene or portion thereof. A portion of a gene of which there are at least two different forms, i.e., two different nucleotide sequences, is referred to as a “polymorphic region of a gene”. A polymorphic locus can be a single nucleotide, the identity of which differs in the other alleles. A polymorphic locus can also be more than one nucleotide long. The allelic form occurring most frequently in a selected population is often referred to as the reference and/or wild-type form. Other allelic forms are typically designated or alternative or variant alleles. Diploid organisms may be homozygous or heterozygous for allelic forms. A diallelic or biallelic polymorphism has two forms. A trialleleic polymorphism has three forms.

A “polymorphic gene” refers to a gene having at least one polymorphic region.

The term “primer” as used herein, refers to a single-stranded oligonucleotide which acts as a point of initiation of template-directed DNA synthesis under appropriate conditions (e.g., in the presence of four different nucleoside triphosphates and as agent for polymerization, such as DNA or RNA polymerase or reverse transcriptase) in an appropriate buffer and at a suitable temperature. The length of a primer may vary but typically ranges from 15 to 30 nucleotides. A primer need not match the exact sequence of a template, but must be sufficiently complementary to hybridize with the template.

The term “primer pair” refers to a set of primers including an upstream primer that hybridizes with the 3′ end of the complement of the DNA sequence to be amplified and a downstream primer that hybridizes with the 3′ end of the sequence to be amplified.

The terms “protein”, “polypeptide” and “peptide” are used interchangeably herein when referring to a gene product.

The term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

A “regulatory element”, also termed herein “regulatory sequence” is intended to include elements which are capable of modulating transcription from a 5′ upstream regulatory sequence, including, but not limited to a basic promoter, and include elements such as enhancers and silencers. The term “enhancer”, also referred to herein as “enhancer element”, is intended to include regulatory elements capable of increasing, stimulating, or enhancing transcription from a 5′ upstream regulatory element, including a basic promoter. The term “silencer”, also referred to herein as “silencer element” is intended to include regulatory elements capable of decreasing, inhibiting, or repressing transcription from a 5′ upstream regulatory element, including a basic promoter. Regulatory elements are typically present in 5′ flanking regions of genes. Regulatory elements also may be present in other regions of a gene, such as introns. Thus, it is possible that CARD4 genes have regulatory elements located in introns, exons, coding regions, and 3′ flanking sequences. Such regulatory elements are also intended to be encompassed by the present invention and can be identified by any of the assays that can be used to identify regulatory elements in 5′ flanking regions of genes.

The term “regulatory element” further encompasses “tissue specific” regulatory elements, i.e., regulatory elements which effect expression of an operably linked DNA sequence preferentially in specific cells (e.g., cells of a specific tissue). Gene expression occurs preferentially in a specific cell if expression in this cell type is significantly higher than expression in other cell types. The term “regulatory element” also encompasses non-tissue specific regulatory elements, i.e., regulatory elements which are active in most cell types. Furthermore, a regulatory element can be a constitutive regulatory element, i.e., a regulatory element which constitutively regulates transcription, as opposed to a regulatory element which is inducible, i.e., a regulatory element which is active primarily in response to a stimulus. A stimulus can be, e.g., a molecule, such as a protein, hormone, cytokine, heavy metal, phorbol ester, cyclic AMP (cAMP), or retinoic acid.

Regulatory elements are typically bound by proteins, e.g., transcription factors. The term “transcription factor” is intended to include proteins or modified forms thereof, which interact preferentially with specific nucleic acid sequences, i.e., regulatory elements, and which in appropriate conditions stimulate or repress transcription. Some transcription factors are active when they are in the form of a monomer. Alternatively, other transcription factors are active in the form of a dimer consisting of two identical proteins or different proteins (heterodimer). Modified forms of transcription factors are intended to refer to transcription factors having a postranslational modification, such as the attachment of a phosphate group. The activity of a transcription factor is frequently modulated by a postranslational modification. For example, certain transcription factors are active only if they are phosphorylated on specific residues. Alternatively, transcription factors can be active in the absence of phosphorylated residues and become inactivated by phosphorylation. A list of known transcription factors and their DNA binding site can be found, e.g., in public databases, e.g., TFMATRIX Transcription Factor Binding Site Profile database.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site.

SNP's may occur in protein-coding nucleic acid sequences, in which case they may give rise to a defective or otherwise variant protein, or genetic disease. Such a SNP may alter the coding sequence of the gene and therefore specify another amino acid (a “missense” SNP) or a SNP may introduce a stop codon (a “nonsense” SNP). When a SNP does not alter the amino acid sequence of a protein, the SNP is called “silent.” SNP's may also occur in noncoding regions of the nucleotide sequence. This may result in defective protein expression, e.g., as a result of alternative spicing, or it may have no effect.

As used herein, the term “specifically hybridizes” or “specifically detects” refers to the ability of a nucleic acid molecule of the invention to hybridize to at least approximately 6, 12, 20, 30, 40, 50, 60, 70, 80, 90, 100, 110, 120, 130 or 140 consecutive nucleotides of either strand of a CARD4 gene.

The term “CARD4 therapeutic” refers to various forms of CARD4 protein or polypeptides, as well as peptidomimetics, nucleic acids, or small molecules, which can modulate at least one activity of a CARD4 protein by mimicking or potentiating (agonizing) or inhibiting (antagonizing) the effects of a naturally-occurring CARD4 protein. A CARD4 therapeutic which mimics or potentiates the activity of a wild-type CARD4 protein is a “CARD4 agonist”. Conversely, a CARD4 therapeutic which inhibits the activity of a wild-type CARD4 protein is a “CARD4 antagonist”. CARD4 therapeutics can be used to treat diseases which are associated with a specific CARD4 allele which encodes a protein having an amino acid sequence that differs from that of the wild-type CARD4 protein.

As used herein, the term “transfection” means the introduction of a nucleic acid, e.g. an expression vector, into a recipient cell by nucleic acid-mediated gene transfer. The term “transduction” is generally used herein when the transfection with a nucleic acid is by viral delivery of the nucleic acid. “Transformation”, as used herein, refers to a process in which a cell's genotype is changed as a result of the cellular uptake of exogenous DNA or RNA, and, for example, the transformed cell expresses a recombinant form of a polypeptide or, in the case of anti-sense expression from the transferred gene, the expression of a naturally-occurring form of the recombinant protein is disrupted.

As used herein, the term “transgene” refers to a nucleic acid sequence which has been genetic-engineered into a cell. Daughter cells deriving from a cell in which a transgene has been introduced are also said to contain the transgene (unless it has been deleted). A transgene can encode, e.g., a polypeptide, or an antisense transcript, partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). Alternatively, a transgene can also be present in an episome. A transgene can include one or more transcriptional regulatory sequence and any other nucleic acid, (e.g. intron), that may be necessary for optimal expression of a selected nucleic acid.

A “transgenic animal” refers to any animal, preferably a non-human animal, e.g. a mammal, bird or an amphibian, in which one or more of the cells of the animal contain heterologous nucleic acid introduced by genetic engineering, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of a protein, e.g. either agonistic or antagonistic forms. However, transgenic animals in which the recombinant gene is silent are also contemplated, as for example, the FLP or CRE recombinase dependent constructs described below. Moreover, “transgenic animal” also includes those recombinant animals in which gene disruption of one or more genes is caused by human intervention, including both recombination and antisense techniques.

The term “treatment,” or “treating” as used herein, is defined as the application or administration of a therapeutic agent to a subject, implementation of lifestyle changes (e.g., changes in diet or environment), administration of medication, or application or administration of a therapeutic agent to a patient who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

As used herein, the term “vector” refers to a nucleic acid molecule capable of transporting or replicating another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively-linked are referred to herein as “expression vectors”. In general, expression vectors of utility in recombinant DNA techniques are often in the form of “plasmids” which refer generally to circular double stranded DNA circles which, in their vector form are not physically linked to the host chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The term “wild-type allele” refers to an allele of a gene which, when present in two copies in a patient results in a wild-type phenotype. There can be several different wild-type alleles of a specific gene, since certain nucleotide changes in a gene may not affect the phenotype of a patient having two copies of the gene with the nucleotide changes. The terms “wild-type” and “reference sequence” are used interchangeably herein.

Polymorphisms of the Invention

The nucleic acid molecules of the invention include specific CARD4 allelic variants. The preferred nucleic acid molecules of the present invention comprise CARD4 sequences having one or more of the polymorphisms shown in Table 1, and those in linkage disequilibrium therewith. Nucleic acid molecules of the invention can function as probes or primers, e.g., in methods for determining the allelic identity of a CARD4 polymorphic region. The nucleic acids of the invention can also be used to determine whether a patient is or is not at risk of developing a disease associated with a specific allelic variant of a CARD4 polymorphic region, e.g., a disease or disorder associated with an aberrant CARD4 activity or expression. The nucleic acids of the invention can further be used to prepare or express CARD4 polypeptides encoded by specific alleles, such as mutant alleles. Such nucleic acids can be used in gene therapy. Polypeptides encoded by specific CARD4 alleles, such as mutant CARD4 polypeptides, can also be used in therapy or for preparing reagents, e.g., antibodies, for detecting CARD4 proteins encoded by these alleles. Accordingly, such reagents can be used to detect mutant CARD4 proteins.

As described herein, several allelic variants of human CARD4 genes have been identified. The invention is intended to encompass all of these allelic variants as well as, those in linkage disequilibrium which can be identified, e.g., according to the methods described herein. “Linkage disequilibrium” refers to an association between specific alleles at two marker loci within a particular population. In general, linkage disequilbrium decreases with an increase in physical distance. If linkage disequilbrium exists between two markers within one gene, then the genotypic information at one marker can be used to make predictions about the genotype of the second marker.

The invention also provides isolated nucleic acids comprising at least one polymorphic region of a CARD4 gene having a nucleotide sequence which differs from the reference nucleotide sequence set forth in SEQ ID NO: 1, or SEQ ID NO: 2.

The nucleic acid molecules of the invention can be single stranded DNA (e.g., an oligonucleotide), double stranded DNA (e.g., double stranded oligonucleotide) or RNA. Preferred nucleic acid molecules of the invention can be used as probes or primers. Primers of the invention refer to nucleic acids which hybridize to a nucleic acid sequence which is adjacent to the region of interest or which covers the region of interest and is extended. As used herein, the term “hybridizes” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions vary according to the length of the involved nucleotide sequence but are known to those skilled in the art and can be found or determined based on teachings in Current Protocols in Molecular Biology, Ausubel et al., eds., John Wiley & Sons, Inc. (1995), sections 2, 4 and 6. Additional stringent conditions and formulas for determining such conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions for hybrids that are at least basepairs in length includes hybridization in 4× sodium chloride/sodium citrate (SSC), at about 65-70° C. (or hybridization in 4X SSC plus 50% formamide at about 42-50° C.) followed by one or more washes in 1×SSC, at about 65-70° C. A preferred, non-limiting example of highly stringent hybridization conditions for such hybrids includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions for such hybrids includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1× SSPE is 0.15 M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete.

The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, T_{m(° C.)=}2(# of A+T bases)+4(# of G+C bases). For hybrids between 18 and 49 base pairs in length, Tₘ(° C.)=81.5+16.6(log₁₀[Na⁺])+0.41(%G+C)−(600/N), where N is the bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5 M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

A primer or probe can be used alone in a detection method, or a primer can be used together with at least one other primer or probe in a detection method. Primers can also be used to amplify at least a portion of a nucleic acid. Probes of the invention refer to nucleic acids which hybridize to the region of interest and which are not further extended. For example, a probe is a nucleic acid which specifically hybridizes to a polymorphic region of a CARD4 gene, and which by hybridization or absence of hybridization to the DNA of a patient or the type of hybrid formed will be indicative of the identity of the allelic variant of the polymorphic region of the CARD4 gene.

Numerous procedures for determining the nucleotide sequence of a nucleic acid molecule, or for determining the presence of mutations in nucleic acid molecules include a nucleic acid amplification step, which can be carried out by, e.g., polymerase chain reaction (PCR). Accordingly, in one embodiment, the invention provides primers for amplifying portions of a CARD4 gene, such as portions of exons and/or portions of introns. In a preferred embodiment, the exons and/or sequences adjacent to the exons of the human CARD4 gene will be amplified to, e.g., detect which allelic variant, if any, of a polymorphic region is present in the CARD4 gene of a patient. Preferred primers comprise a nucleotide sequence complementary a specific allelic variant of a CARD4 polymorphic region and of sufficient length to selectively hybridize with a CARD4 gene. In a preferred embodiment, the primer, e.g., a substantially purified oligonucleotide, comprises a region having a nucleotide sequence which hybridizes under stringent conditions to about 6, 8, 10, or 12, preferably 25, 30, 40, 50, or 75 consecutive nucleotides of a CARD4 gene. In an even more preferred embodiment, the primer is capable of hybridizing to a CARD4 nucleotide sequence and comprises a nucleotide sequence of any sequence set forth in any of SEQ ID NOs: 4-29, or complements thereof. For example, primers comprising a nucleotide sequence of at least about 15 consecutive nucleotides, at least about 25 nucleotides or having from about 15 to about 20 nucleotides set forth in any of SEQ ID NOs: 4-29 or complement thereof are provided by the invention. Primers having a sequence of more than about 25 nucleotides are also within the scope of the invention. Preferred primers of the invention are primers that can be used in PCR for amplifying each of the exons of a CARD4 gene.

Primers can be complementary to nucleotide sequences located close to each other or further apart, depending on the use of the amplified DNA. For example, primers can be chosen such that they amplify DNA fragments of at least about 10 nucleotides or as much as several kilobases. Preferably, the primers of the invention will hybridize selectively to CARD4 nucleotide sequences located about 150 to about 350 nucleotides apart.

For amplifying at least a portion of a nucleic acid, a forward primer (i.e., 5′ primer) and a reverse primer (i.e., 3′ primer) will preferably be used. Forward and reverse primers hybridize to complementary strands of a double stranded nucleic acid, such that upon extension from each primer, a double stranded nucleic acid is amplified. A forward primer can be a primer having a nucleotide sequence or a portion of the nucleotide sequence shown in Table I (SEQ ID NOs: 4-29) or in SEQ ID NOs.: 30-77. A reverse primer can be a primer having a nucleotide sequence or a portion of the nucleotide sequence that is complementary to a nucleotide sequence shown in Table 1 (SEQ ID NOs: 4-29) or in SEQ ID NOs.: 30-77. Preferred pairs of primers for amplifying each of the exons of human CARD4 are set forth in Table 3 (see Example 2).

Yet other preferred primers of the invention are nucleic acids which are capable of selectively hybridizing to an allelic variant of a polymorphic region of a CARD4 gene. Thus, such primers can be specific for a CARD4 gene sequence, so long as they have a nucleotide sequence which is capable of hybridizing to a CARD4 gene. Preferred primers are capable of specifically hybridizing to any of the allelic variants listed in Table 1 (i.e., sequences comprising any of SEQ ID NOs: 4-29 or a complement thereof). Such primers can be used, e.g., in sequence specific oligonucleotide priming as described further herein.

The CARD4 nucleic acids of the invention can also be used as probes, e.g., in therapeutic and diagnostic assays. For instance, the present invention provides a probe comprising a substantially purified oligonucleotide, which oligonucleotide comprises a region having a nucleotide sequence that is capable of hybridizing specifically to a region of a CARD4 gene which is polymorphic (i.e., sequences comprising any of SEQ ID NOs: 4-29 or a complement thereof). In an even more preferred embodiment of the invention, the probes are capable of hybridizing specifically to one allelic variant of a CARD4 gene having a nucleotide sequence which differs from the nucleotide sequence set forth in SEQ ID NO: 1, 2, or 3. Such probes can then be used to specifically detect which allelic variant of a polymorphic region of a CARD4 gene is present in a patient. The polymorphic region can be located in a 5′ untranslated region, a 5′ upstream regulatory element, an exon, an intron, or a 3′ untranslated region of a CARD4 gene.

Particularly, preferred probes of the invention have a number of nucleotides sufficient to allow specific hybridization to the target nucleotide sequence. Where the target nucleotide sequence is present in a large fragment of DNA, such as a genomic DNA fragment of several tens or hundreds of kilobases, the size of the probe may have to be longer to provide sufficiently specific hybridization, as compared to a probe which is used to detect a target sequence which is present in a shorter fragment of DNA. For example, in some diagnostic methods, a portion of a CARD4 gene may first be amplified and thus isolated from the rest of the chromosomal DNA and then hybridized to a probe. In such a situation, a shorter probe will likely provide sufficient specificity of hybridization. For example, a probe having a nucleotide sequence of about 10 nucleotides may be sufficient.

In preferred embodiments, the probe or primer further comprises a label attached thereto, which, e.g., is capable of being detected, e.g. the label group is selected from amongst radioisotopes, fluorescent compounds, enzymes, and enzyme co-factors.

In a preferred embodiment of the invention, the isolated nucleic acid, which is used, e.g., as a probe or a primer, is modified, so as to be more stable than naturally occurring nucleotides. Exemplary nucleic acid molecules which are modified include phosphoramidate, phosphothioate and methylphosphonate analogs of DNA (see also U.S. Pat. Nos. 5,176,996; 5,264,564; and 5,256,775).

The nucleic acids of the invention can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the nucleic acid of the invention may be conjugated to another molecule, e.g., a peptide, hybridization triggered cross-linking agent, transport agent, hybridization-triggered cleavage agent, etc.

The isolated nucleic acid comprising a CARD4 intronic sequence may comprise at least one modified base moiety which is selected from the group including but not limited to 5-fluorouracil, 5-bromouracil, 5-chlorouracil, 5-iodouracil, hypoxanthine, xantine, 4-acetylcytidine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytidine, 5-methylcytidine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytidine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The isolated nucleic acid may also comprise at least one modified sugar moiety selected from the group including but not limited to arabinose, 2-fluoroarabinose, xylulose, and hexose. In yet another embodiment, the nucleic acid comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the nucleic acid is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Any nucleic acid fragment of the invention can be prepared according to methods well known in the art and described, e.g., in Sambrook, Fritsch, and Maniatis (1989) Molecular Cloning: A Laboratory Manual, Cold Spring Harbor Laboratory Press, Cold Spring Harbor, N.Y. For example, discrete fragments of the DNA can be prepared and cloned using restriction enzymes. Alternatively, discrete fragments can be prepared using the Polymerase Chain Reaction (PCR) using primers having an appropriate sequence. Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. ((1988) Nucl. Acids Res. 16:3209), methylphosphonate oligonucleotides can be prepared by use of controlled pore glass polymer supports (Sarin et al. (1988) Proc. Natl. Acad. Sci. U.S.A. 85:7448-7451), etc.

The invention also provides vectors and plasmids comprising the nucleic acids of the invention. For example, in one embodiment, the invention provides a vector comprising at least a portion of a CARD4 gene comprising a polymorphic region. Thus, the invention provides vectors for expressing at least a portion of the newly identified allelic variants of the human CARD4 gene reference, as well as other allelic variants, comprising a nucleotide sequence which is different from the nucleotide sequence disclosed in GI 4156149, or GI 11419372. The allelic variants can be expressed in eukaryotic cells, e.g., cells of a patient, or in prokaryotic cells.

In one embodiment, the vector comprising at least a portion of a CARD4 allele is introduced into a host cell, such that a protein encoded by the allele is synthesized. The CARD4 protein produced can be used, e.g., for the production of antibodies, which can be used, e.g., in methods for detecting mutant forms of CARD4. Alternatively, the vector can be used for gene therapy, and be, e.g., introduced into a patient to produce CARD4 protein. Host cells comprising a vector having at least a portion of a CARD4 gene are also within the scope of the invention.

Methods

The invention further provides predictive medicine methods, which are based, at least in part, on the discovery of CARD4 polymorphisms which are associated with specific physiological states and/or diseases or disorders.

For example, information obtained using the diagnostic assays described herein is useful for determining that a patient suffering from or at risk for an inflammatory or allergy disease or disorder, e.g., asthma, has a more or less severe disease phenotype, e.g., a more or less severe asthma phenotype. Alternatively, the information can be used prognostically for predicting whether a patient will be responsive to treatment of an inflammatory or allergy or apoptotic disease or disorder, including, but not limited to, asthma, with a CARD4 inhibitor or other related agent. Based on the prognostic information, a health care provider can recommend a regimen (e.g., diet or exercise) or therapeutic protocol, (e.g., administration of a CARD4 inhibitor) useful for preventing the particular disease or disorder, or symptoms of the disease or disorder in the patient e.g., asthma.

In addition, knowledge of the identity of a particular CARD4 allele in an individual (the CARD4 genetic profile, including the presence or absence of the polymorphisms described herein), alone or in conjunction with information on other genetic defects contributing to the same disease (the genetic profile of the particular disease, e.g., asthma) allows customization of therapy for a particular disease to the individual's genetic profile. For example, an individual's CARD4 genetic profile or the genetic profile of a disease or condition associated with a specific allele of a CARD4 polymorphic region, can enable a health care provider: 1) to more effectively prescribe a drug that will address the molecular basis of the disease or condition, and 2) to better determine the appropriate dosage of a particular drug. For example, the expression level of CARD4 proteins, alone or in conjunction with the expression level of other genes, known to contribute to the same disease, can be measured in many patients at various stages of the disease to generate a transcriptional or expression profile of the disease. Expression patterns of individual patients can then be compared to the expression profile of the disease to determine the appropriate drug and dose to administer to the patient.

The ability to target populations expected to show the highest clinical benefit, e.g., subgroups of asthmatic populations which respond or do not respond to specific therapies, or subgroups of asthmatic populations with more or less severe asthma phenotypes, based on the CARD4 or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling (e.g., since the use of CARD4 as a marker is useful for optimizing effective dose).

These and other methods are described in further detail in the following sections.

A. Prognostic and Diagnostic Assays

The present methods provide means for determining if a patient has a disease, condition or disorder that is associated a specific CARD4 allele, or the severity of an inflammatory, apoptotic or allergic disease or disorder phenotype. The present methods also provide means for predicting or determining if a patient will be more or less responsive to treatment with a therapy that is impacted by CARD-4, based on determination of a specific CARD4 allele.

The present invention provides methods for determining the molecular structure of a CARD4 gene, such as a human CARD4 gene, or a portion thereof. For example, the present invention provides methods for determining the presence or absence of the polymorphism described herein. In one embodiment, determining the molecular structure of at least a portion of a CARD4 gene comprises determining the identity of the allelic variant of at least one polymorphic region of a CARD4 gene (determining the presence or absence of one or more of the allelic variants, or their complements, of SEQ ID NOs.: 4-29). A polymorphic region of a CARD4 gene can be located in an exon, an intron, at an intron/exon border, or in a 5′ or 3′ untranslated region, including a regulatory region.

The invention provides methods for determining whether a patient has a specific disease or disorder phenotype, e.g., asthma phenotype, associated with a specific allelic variant of a polymorphic region of a CARD4 gene. Such disease phenotypes are associated with aberrant CARD4 activity, e.g., aberrant CARD4 expression or activity. Aberrant CARD4 protein level can result from aberrant transcription or post transcriptional regulation. Thus, allelic differences in specific regions of a CARD4 gene can result in differences of CARD4 protein due to differences in regulation of expression. In particular, some of the identified polymorphisms in the human CARD4 gene may be associated with differences in the level of transcription, RNA maturation, splicing, or translation of the CARD4 gene or transcription product.

In preferred embodiments, the methods of the invention can be characterized as comprising detecting, in a sample of cells or nucleic acid from the patient, the presence or absence of a specific allelic variant of one or more polymorphic regions of a CARD4 gene. The allelic differences can be: (i) a difference in the identity of at least one nucleotide or (ii) a difference in the number of nucleotides, which difference can be a single nucleotide or several nucleotides. The invention also provides methods for detecting differences in CARD4 genes such as chromosomal rearrangements, e.g., chromosomal dislocation. The invention can also be used in prenatal diagnostics.

A preferred detection method is allele specific hybridization using probes overlapping the polymorphic site and having about 5, 10, 20, 25, or 30 nucleotides around the polymorphic region. In a preferred embodiment of the invention, several probes capable of hybridizing specifically to allelic variants are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment. For example, the identity of the allelic variant of the nucleotide polymorphism in the 5′ upstream regulatory element can be determined in a single hybridization experiment.

In other detection methods, it is necessary to first amplify at least a portion of a CARD4 gene prior to identifying the allelic variant. Amplification can be performed, e.g., by PCR and/or LCR (see Wu and Wallace (1989) Genomics 4:560), according to methods known in the art. In one embodiment, genomic DNA of a cell is exposed to two PCR primers and amplification for a number of cycles sufficient to produce the required amount of amplified DNA. In preferred embodiments, the primers are located between 150 and 350 base pairs apart. Preferred primers, such as primers for amplifying each of the exons of the human CARD4 gene, are listed in Table 3.

Alternative amplification methods include: self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), and self-sustained sequence replication (Guatelli et al., (1989) Proc. Nat. Acad. Sci. 87:1874), and nucleic acid based sequence amplification (NABSA), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment, any of a variety of sequencing reactions known in the art can be used to directly sequence at least a portion of a CARD4 gene and detect allelic variants, e.g., mutations, by comparing the sequence of the sample sequence with the corresponding wild-type (control) sequence. Exemplary sequencing reactions include those based on techniques developed by Maxam and Gilbert (Proc. Natl Acad Sci USA (1977) 74:560) or Sanger (Sanger et al (1977) Proc. Nat. Acad. Sci 74:5463). It is also contemplated that any of a variety of automated sequencing procedures may be utilized when performing the subject assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by Köster; U.S. Pat. No. 5,547,835 and international patent application Publication Number WO 94/21822 entitled DNA Sequencing by Mass Spectrometry Via Exonuclease Degradation by Köster), and U.S Pat. No. 5,605,798 and International Patent Application No. PCT/US96/03651 entitled DNA Diagnostics Based on Mass Spectrometry by Köster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction.

Yet other sequencing methods are disclosed, e.g., in U.S. Pat. No. 5,580,732 and U.S. Pat. No. 5,571,676.

In some cases, the presence of a specific allele of a CARD4 gene in DNA from a patient can be shown by restriction enzyme analysis. For example, a specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (Myers et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of a CARD4 allelic variant with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, Cotton et al. (1988) Proc. Natl Acad Sci USA 85:4397; Saleeba et al. (1992) Methods Enzymol. 217:286-295. In a preferred embodiment, the control or sample nucleic acid is labeled for detection.

In another embodiment, an allelic variant can be identified by denaturing high-performance liquid chromatography (DHPLC) (Oefner and Underhill (1995) Am. J. Human Gen. 57:Suppl. A266). DHPLC uses reverse-phase ion-pairing chromatography to detect the heteroduplexes that are generated during amplification of PCR fragments from individuals who are heterozygous at a particular nucleotide locus within that fragment (Oefner and Underhill (1995) Am. J Human Gen. 57:Suppl. A266). In general, PCR products are produced using PCR primers flanking the DNA of interest. DHPLC analysis is carried out and the resulting chromatograms are analyzed to identify base pair alterations or deletions based on specific chromatographic profiles (see O'Donovan et al. (1998) Genomics 52:44-49).

In other embodiments, alterations in electrophoretic mobility is used to identify the type of CARD4 allelic variant. For example, single strand conformation polymorphism (SSCP) may be used to detect differences in electrophoretic mobility between mutant and wild type nucleic acids (Orita et al. (1989) Proc Natl. Acad. Sci USA 86:2766, see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the identity of an allelic variant of a polymorphic region is obtained by analyzing the movement of a nucleic acid comprising the polymorphic region in polyacrylamide gels containing a gradient of denaturant is assayed using denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between two nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163; Saiki et al (1989) Proc. Natl Acad. Sci USA 86:6230; and Wallace et al. (1979) Nucl. Acids Res. 6:3543). Such allele specific oligonucleotide hybridization techniques may be used for the simultaneous detection of several nucleotide changes in different polylmorphic regions of CARD4. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al (1992) Mol. Cell Probes 6:1).

In another embodiment, identification of the allelic variant is carried out using an oligonucleotide ligation assay (OLA), as described, e.g., in U.S. Pat. No. 4,998,617 and in Landegren et al. (1988) Science 241:1077-1080. The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson et al. (1990) Proc. Natl. Acad. Sci. (U.S.A.) 87:8923. In this method, PCR is used to achieve the exponential amplification of target DNA, which is then detected using OLA.

Several techniques based on this OLA method have been developed and can be used to detect specific allelic variants of a polymorphic region of a CARD4 gene. For example, U.S. Pat. No. 5,593,826 discloses an OLA using an oligonucleotide having 3'-amino group and a 5'-phosphorylated oligonucleotide to form a conjugate having a phosphoramidate linkage. In another variation of OLA described in Tobe et al. ((1996) Nucleic Acids Res 24:3728), OLA combined with PCR permits typing of two alleles in a single microtiter well. By marking each of the allele-specific primers with a unique hapten, i.e., digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

The invention further provides methods for detecting single nucleotide polymorphisms in a CARD4 gene. Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient. Several methods have been developed to facilitate the analysis of such single nucleotide polymorphisms.

In one embodiment, the single base polymorphism can be detected by using a specialized exonuclease-resistant nucleotide, as disclosed, e.g., in U.S. Pat. No. 4,656,127. According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen et al. (French Patent 2,650,840; PCT Appln. No. WO91/02087). As in the method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet et al. (PCT Appln. No. 92/15712). The method of Goelet et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet et al. is preferably a heterogeneous phase assay, in which the primer or the target molecule is immobilized to a solid phase.

Recently, several primer-guided nucleotide incorporation procedures for assaying polymorphic sites in DNA have been described (Komher et al. (1989) Nucl. Acids. Res. 17:7779-7784; Sokolov (1990) Nucl. Acids Res. 18:3671; Syvanen et al. (1990) Genomics 8:684-692; Kuppuswamy et al. (1991) Proc. Natl. Acad. Sci. (U.S.A.) 88:1143-1147; Prezant et al. (1992) Hum. Mutat. 1:159-164; Ugozzoli et al. (1992) GATA 9:107-112; Nyren et al.,(1993) Anal. Biochem. 208:171-175). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen et al. (1993) Amer. J. Hum. Genet. 52:46-59).

If a polymorphic region is located in an exon, either in a coding or non-coding portion of the gene, the identity of the allelic variant can be determined by determining the molecular structure of the mRNA, pre-mRNA, or cDNA. The molecular structure can be determined using any of the above described methods for determining the molecular structure of the genomic DNA, e.g., see Example 1.

The methods described herein may be performed, for example, by utilizing pre-packaged diagnostic kits, such as those described above, comprising at least one probe or primer nucleic acid described herein, which may be conveniently used, e.g., to determine whether a patient has a specific disease phenotype associated with a specific CARD4 allelic variant.

Sample nucleic acid to be analyzed by any of the above-described diagnostic and prognostic methods can be obtained from any cell type or tissue of a patient. For example, a patient's bodily fluid (e.g., blood) can be obtained by known techniques (e.g. venipuncture). Alternatively, nucleic acid tests can be performed on dry samples (e.g. hair or skin). Fetal nucleic acid samples can be obtained from maternal blood as described in International Patent Application No. WO91/07660 to Bianchi. Alternatively, amniocytes or chorionic villi may be obtained for performing prenatal testing.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo (1992), PCR in situ Hybridization: Protocols and Applications, Raven Press, NY).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

B. Pharmacogenomics

Knowledge of the identity of the allele of one or more CARD4 gene polymorphic regions in a patient (the CARD4 genetic profile), alone or in conjunction with information on other genetic defects contributing to the same disease (the genetic profile of the particular disease.

For example, patients having a specific allele of a CARD4 gene may or may not exhibit symptoms of a particular disease or be predisposed to developing symptoms of a particular disease. Further, if those patients are symptomatic, they may or may not respond well to a certain drug, e.g., a specific CARD4 therapeutic, such as a CARD4 inhibitor, but may respond to another. Thus, generation of a CARD4 genetic profile (e.g., categorization of alterations in CARD4 genes which are associated with the development of a particular disease, including determination of the presence of the haplotype described herein), from a population of patients, who are symptomatic for a disease or condition that is caused by or contributed to by a defective and/or aberrantly expressed CARD4 gene and/or protein (a CARD4 genetic population profile) and comparison of an individual's CARD4 profile to the population profile, permits the selection or design of drugs that are expected to be safe and efficacious for a particular patient or patient population (i.e., a group of patients having the same genetic alteration).

For example, a CARD4 population profile can be performed by determining the CARD4 profile, e.g., the identity of CARD4 alleles, in particular the identity of CARD4 alleles included in the haplotype as described herein, in a patient population having a disease, which is associated with one or more specific alleles of CARD4 polymorphic regions. Optionally, the CARD4 population profile can further include information relating to the response of the population to a CARD4 therapeutic, using any of a variety of methods, including, monitoring: 1) the severity of symptoms associated with the CARD4 related disease; 2) CARD4 gene expression level; 3) CARD4 mRNA level; 4) CARD4 protein level; and/or 5) CARD4 activity level, and dividing or categorizing the population based on particular CARD4 alleles. The CARD4 genetic population profile can also, optionally, indicate those particular CARD4 alleles which are present in patients that are either responsive or non-responsive to a particular therapeutic, e.g., a CARD4 inhibitor. This information or population profile, is then useful for predicting which individuals should respond to particular drugs, based on their individual CARD4 profile.

In a preferred embodiment, the CARD4 profile is a transcriptional or expression level profile and is comprised of determining the expression level of CARD4 proteins, alone or in conjunction with the expression level of other genes known to contribute to the same disease at various stages of the disease.

Pharmacogenomic studies can also be performed using transgenic animals. For example, one can produce transgenic mice, e.g., as described herein, which contain a specific allelic variant of a CARD4 gene. These mice can be created, e.g., by replacing their wild-type CARD4 gene with an allele of the human CARD4 gene. The response of these mice to specific CARD4 therapeutics can then be determined.

C. Monitoring Effects of CARD4 Therapeutics During Clinical Trials

The present invention provides a method for monitoring the effectiveness of treatment of a patient with an agent (e.g., an agonist, antagonist, peptidomimetic, protein, peptide, nucleic acid, small molecule, or other drug candidate identified, e.g., by the screening assays described herein) comprising the steps of (i) obtaining a preadministration sample from a patient prior to administration of the agent; (ii) detecting the level of expression or activity of a CARD4 protein, mRNA or gene in the preadministration sample; (iii) obtaining one or more post-administration samples from the patient; (iv) detecting the level of expression or activity of the CARD4 protein, mRNA or gene in the post-administration samples; (v) comparing the level of expression or activity of the CARD4 protein, mRNA, or gene in the preadministration sample with those of the CARD4 protein, mRNA, or gene in the post administration sample or samples; and (vi) altering the administration of the agent to the patient accordingly. For example, increased administration of the agent may be desirable to increase (or decrease) the expression or activity of CARD4 to higher levels than detected, i.e., to increase the effectiveness of the agent. Alternatively, decreased administration of the agent may be desirable to increase (or decrease) expression or activity of CARD4 to lower levels than detected, i.e., to decrease the effectiveness of the agent.

Cells of a patient may also be obtained before and after administration of a CARD4 therapeutic to detect the level of expression of genes other than CARD4, to verify that the CARD4 therapeutic does not increase or decrease the expression of genes which could be deleterious. This can be done, e.g., by using the method of transcriptional profiling. Thus, mRNA from cells exposed in vivo to a CARD4 therapeutic and mRNA from the same type of cells that were not exposed to the CARD4 therapeutic could be reverse transcribed and hybridized to a chip containing DNA from numerous genes, to thereby compare the expression of genes in cells treated and not treated with a CARD4 therapeutic. If, for example a CARD4 therapeutic turns on the expression of a proto-oncogene in an individual, use of this particular CARD4 therapeutic may be undesirable.

D. Methods of Treatment

The present invention provides for both prophylactic and therapeutic methods of treating a patient having a disorder associated with specific CARD4 alleles and/or aberrant CARD4 expression or activity, e.g., inflammatory or allergy diseases or disorders, such as asthma.

i) Prophylactic Methods

In one aspect, the invention provides a method for preventing in a patient, a disease or condition associated with a specific CARD4 allele such as an inflammatory or allergy disease or disorder, e.g., asthma, and medical conditions resulting therefrom, by administering to the patient an agent which counteracts the unfavorable biological effect of the specific CARD4 allele. Subjects at risk for such a disease can be identified by a diagnostic or prognostic assay, e.g., as described herein. Administration of a prophylactic agent can occur prior to the manifestation of symptoms associated with specific CARD4 alleles, such that a disease or disorder is prevented or, alternatively, delayed in its progression. Depending on the identity of the CARD4 allele in a patient, a compound that counteracts the effect of this allele is administered. The compound can be a compound modulating the activity of CARD4, e.g., a CARD4 inhibitor or a CARD4 activator. The treatment can also be a specific diet, or environmental alteration. In particular, the treatment can be undertaken prophylactically, before any other symptoms are present. Such a prophylactic treatment could thus prevent the development of an aberrant inflammatory response, e.g., asthma. The prophylactic methods are similar to therapeutic methods of the present invention and are further discussed in the following subsections.

(ii) Therapeutic Methods

The invention further provides methods of treating patients having a specific disease or disorder phenotype associated with a specific allelic variant of a polymorphic region of a CARD4 gene, e.g., a more moderate or more severe phenotype.

In one embodiment, the method comprises (a) determining the identity of one or more allelic variants of the invention; and (b) administering to the patient a compound that compensates for the effect of the specific allelic variant. The polymorphic region can be localized at any location of the gene, e.g., in a regulatory element (e.g., in a 5′ upstream regulatory element), in an exon, (e.g., coding region of an exon), in an intron, or at an exon/intron border. Thus, depending on the site of the polymorphism in the CARD4 gene, a patient having a specific variant of the polymorphic region which is associated with a specific disease or condition, can be treated with compounds which specifically compensate for the effect of the allelic variant.

Generally, the allelic variant can be a mutant allele, i.e., an allele which when present in one, or preferably two copies, in a patient results in a change in the phenotype of the patient. A mutation can be a substitution, deletion, and/or addition of at least one nucleotide relative to the wild-type allele (i.e., the reference sequence). Depending on where the mutation is located in the CARD4 gene, the patient can be treated to specifically compensate for the mutation. For example, if the mutation is present in the coding region of the gene and results in a more active CARD4 protein, the patient can be treated, e.g., by administration to the patient of a CARD4 inhibitor. Normal CARD4 protein can also be used to counteract or compensate for the endogenous mutated form of the CARD4 protein. Normal CARD4 protein can be directly delivered to the patient or indirectly by gene therapy wherein some cells in the patient are transformed or transfected with an expression construct encoding wild-type CARD4 protein. Nucleic acids encoding wild-type human CARD4 protein are set forth in SEQ ID NOs.: 1 and 2 (GenBank® Identification Nos. GI 4156149 and GI 11419372).

Yet in another embodiment, the invention provides methods for treating a patient having a mutated CARD4 gene, in which the mutation is located in a regulatory region of the gene. Such a regulatory region can be localized in the 5′ upstream regulatory element of the gene, in the 5′ or 3′ untranslated region of an exon, or in an intron. A mutation in a regulatory region can result in increased production of CARD4 protein, decreased production of CARD4 protein, or production of CARD4 having an aberrant tissue distribution. The effect of a mutation in a regulatory region upon the CARD4 protein can be determined, e.g., by measuring the CARD4 protein level or mRNA level in cells having a CARD4 gene having this mutation and which, normally (i.e., in the absence of the mutation) produce CARD4 protein. The effect of a mutation can also be determined in vitro. For example, if the mutation is in the 5′ upstream regulatory element, a reporter construct can be constructed which comprises the mutated 5′ upstream regulatory element linked to a reporter gene, the construct transfected into cells, and comparison of the level of expression of the reporter gene under the control of the mutated 5′ upstream regulatory element and under the control of a wild-type 5′ upstream regulatory element. Such experiments can also be carried out in mice transgenic for the mutated 5′ upstream regulatory element. If the mutation is located in an intron, the effect of the mutation can be determined, e.g., by producing transgenic animals in which the mutated CARD4 gene has been introduced and in which the wild-type gene may have been knocked out. Comparison of the level of expression of CARD4 in the mice transgenic for the mutant human CARD4 gene with mice transgenic for a wild-type human CARD4 gene will reveal whether the mutation results in increased, or decreased synthesis of the CARD4 protein and/or aberrant tissue distribution of CARD4 protein. Such analysis could also be performed in cultured cells, in which the human mutant CARD4 gene is introduced and, e.g., replaces the endogenous wild-type CARD4 gene in the cell. Thus, depending on the effect of the mutation in a regulatory region of a CARD4 gene, a specific treatment can be administered to a patient having such a mutation.

A correlation between drug responses and specific alleles of CARD4 can be shown, for example, by clinical studies wherein the response to specific drugs of patients having different allelic variants of a polymorphic region of a CARD4 gene is compared. Such studies can also be performed using animal models, such as mice having various alleles of human CARD4 genes and in which, e.g., the endogenous CARD4 has been inactivated such as by a knock-out mutation. Test drugs are then administered to the mice having different human CARD4 alleles and the response of the different mice to a specific compound is compared. Accordingly, the invention provides assays for identifying the drug which will be best suited for treating a specific disease or condition in a patient. For example, it will be possible to select drugs which will be devoid of toxicity, or have the lowest level of toxicity possible for treating a patient having a disease or condition.

Other Uses For the Nucleic Acid Molecules of the Invention

The identification of different alleles of CARD4 can also be useful for identifying an individual among other individuals from the same species. For example, DNA sequences can be used as a fingerprint for detection of different individuals within the same species (Thompson and Thompson, eds., Genetics in Medicine, W B Saunders Co., Philadelphia, Pa. (1991)). This is useful, for example, in forensic studies and paternity testing, as described below.

A. Forensics

Determination of which specific allele occupies a set of one or more polymorphic sites in an individual identifies a set of polymorphic forms that distinguish the individual from others in the population. See generally National Research Council, The Evaluation of Forensic DNA Evidence (Eds. Pollard et al., National Academy Press, DC, 1996). The more polymorphic sites that are analyzed, the lower the probability that the set of polymorphic forms in one individual is the same as that in an unrelated individual. Preferably, if multiple sites are analyzed, the sites are unlinked. Thus, the polymorphisms of the invention can be used in conjunction with known polymorphisms in distal genes. Preferred polymorphisms for use in forensics are biallelic because the population frequencies of two polymorphic forms can usually be determined with greater accuracy than those of multiple polymorphic forms at multi-allelic loci.

The capacity to identify a distinguishing or unique set of polymorphic markers in an individual is useful for forensic analysis. For example, one can determine whether a blood sample from a suspect matches a blood or other tissue sample from a crime scene by determining whether the set of polymorphic forms occupying selected polymorphic sites is the same in the suspect and the sample. If the set of polymorphic markers does not match between a suspect and a sample, it can be concluded (barring experimental error) that the suspect was not the source of the sample. If the set of markers is the same in the sample as in the suspect, one can conclude that the DNA from the suspect is consistent with that found at the crime scene. If frequencies of the polymorphic forms at the loci tested have been determined (e.g., by analysis of a suitable population of individuals), one can perform a statistical analysis to determine the probability that a match of suspect and crime scene sample would occur by chance.

p(ID) is the probability that two random individuals have the same polymorphic or allelic form at a given polymorphic site. For example, in biallelic loci, four genotypes are possible: AA, AB, BA, and BB. If alleles A and B occur in a haploid genome of the organism with frequencies x and y, the probability of each genotype in a diploid organism is (see WO 95/12607):

Homozygote: p(AA)=x²

Homozygote: p(BB)=y²=(1−x)²

Single Heterozygote: p(AB)=p(BA)=xy=x(1−x)

Both Heterozygotes: p(AB+BA)=2xy=2x(1−x) The probability of identity at one locus (i.e., the probability that two individuals, picked at random from a population will have identical polymorphic forms at a given locus) is given by the equation: p(ID)=(x²).

These calculations can be extended for any number of polymorphic forms at a given locus. For example, the probability of identity p(ID) for a 3-allele system where the alleles have the frequencies in the population of x, y, and z, respectively, is equal to the sum of the squares of the genotype frequencies: P(ID)=x⁴+(2xy)²+(2yz)²+(2xz)²+z⁴+y⁴.

In a locus of n alleles, the appropriate binomial expansion is used to calculate p(ID) and p(exc).

The cumulative probability of identity (cum p(ID)) for each of multiple unlinked loci is determined by multiplying the probabilities provided by each locus:

cum p(ID)=p(ID1)p(ID2)p(ID3) . . . p(IDn).

The cumulative probability of non-identity for n loci (i.e., the probability that two random individuals will be difference at 1 or more loci) is given by the equation: cum p(nonID)=1−cum p(ID).

If several polymorphic loci are tested, the cumulative probability of non-identity for random individuals becomes very high (e.g., one billion to one). Such probabilities can be taken into account together with other evidence in determining the guilt or innocence of the suspect.

B. Paternity Testing

The object of paternity testing is usually to determine whether a male is the father of a child. In most cases, the mother of the child is known, and thus, it is possible to trace the mother's contribution to the child's genotype. Paternity testing investigates whether the part of the child's genotype not attributable to the mother is consistent to that of the puntative father. Paternity testing can be performed by analyzing sets of polymorphisms in the putative father and in the child.

If the set of polymorphisms in the child attributable to the father does not match the set of polymorphisms of the putative father, it can be concluded, barring experimental error, that that putative father is not the real father. If the set of polymorphisms in the child attributable to the father does match the set of polymorphisms of the putative father, a statistical calculation can be performed to determine the probability of a coincidental match. The probability of parentage exclusion (representing the probability that a random male will have a polymorphic form at a given polymorphic site that makes him incompatible as the father) region, a 5′ upstream regulatory element, an exon, an intron, or a 3′ untranslated region of a is given by the equation (see WO 95/12607): p(exc)=xy(1−xy), where x and y are the population frequencies of alleles A and B of a biallelic polymorphic site. (At a triallelic site p(exc)=xy(1−xy)+yz(1−yz)+xz(1−xz)+3xyz(1-xyz)), where x, y, and z and the respective populations frequencies of alleles A, B, and C).

The probability of non-exclusion is: p(non-exc)=1−p(exc).

The cumulative probability of non-exclusion (representing the values obtained when n loci are is used) is thus:

Cum p(non−exc)=p(non−exc1)p(non−exc2)p(non−exc3) . . . p(non-excn). The cumulative probability of the exclusion for n loci (representing the probability that a random male will be excluded: cum p(exc)=1−cum p(non−exc). If several polymorphic loci are included in the analysis, the cumulative probability of exclusion of a random male is very high. This probability can be taken into account in assessing the liability of a putative father whose polymorphic marker set matches the child's polymorphic marker set attributable to his or her father.

Kits

As set forth herein, the invention provides methods, e.g., diagnostic and therapeutic methods, e.g., for determining the type of allelic variant of a polymorphic region present in a CARD4 gene, such as a human CARD4 gene. In preferred embodiments, the methods use probes or primers comprising nucleotide sequences which are complementary polymorphic region of a CARD4 gene (SEQ ID Nos: 4-29). In a more preferred embodiment, the methods are used to determine the identity of one or more allelic variants described herein. Accordingly, the invention provides kits for performing these methods.

In a preferred embodiment, the invention provides a kit for determining whether a patient has a more moderate or more severe inflammatory or allergy disease phenotype associated with a specific allelic variant of a CARD4 polymorphic region. In an even more preferred embodiment, the disease or disorder is characterized by an abnormal CARD4 activity, e.g., aberrant CARD4 expression. In an even more preferred embodiment, the inflammatory or allergy disease is, e.g., asthma, lung inflammation, nephritis, amyloidosis, rheumatoid arthritis, chronic bronchitis, sarcoidosis, scleroderma, lupus, polymyositis, Reiter's syndrome, psoriasis, pelvic inflammatory disease, orbital inflammatory disease, thrombotic disease, and inappropriate allergic responses to environmental stimuli such as poison ivy, pollen, insect stings and certain foods, including atopic dermatitis and contact dermatitis, multiple sclerosis and Crohn's disease, chronic obstructive pulmonary disease, inflammatory bowel disease, or psoriasis

A preferred kit provides reagents for determining whether a patient will or will not be responsive to treatment of a disease or disorder associated with a specific allelic variant of a polymorphic region of a CARD4 gene, e.g., asthma. In a preferred embodiment, the inflammatory disease is, e.g., asthma, lung inflammation, nephritis, amyloidosis, rheumatoid arthritis, chronic bronchitis, sarcoidosis, scleroderma, lupus, polymyositis, Reiter's syndrome, psoriasis, pelvic inflammatory disease, orbital inflammatory disease, thrombotic disease, and inappropriate allergic responses to environmental stimuli such as poison ivy, pollen, insect stings and certain foods, including atopic dermatitis and contact dermatitis, multiple sclerosis and Crohn's disease, chronic obstructive pulmonary disease, rheumatoid arthritis, inflammatory bowel disease, or psoriasis. In a preferred embodiment, the kit of the invention can be used in selecting the appropriate drug to administer to a patient suffering from an inflammatory disease or disorder.

Preferred kits comprise at least one probe or primer which is capable of specifically hybridizing under stringent conditions to a CARD4 sequence or polymorphic region and instructions for use. The kits preferably comprise at least one of the above described nucleic acids. Preferred kits for amplifying at least a portion of a CARD4 gene, e.g., the 5′ upstream regulatory element, comprise two primers, at least one of which is capable of hybridizing to an allelic variant sequence. Even more preferred kits comprise a pair of primers selected from the group consisting of SEQ ID NO: 30 and SEQ ID NO: 54, SEQ ID NO: 31 and SEQ ID NO: 55, SEQ ID NO: 32 and SEQ ID NO: 56, SEQ ID NO: 33 and SEQ ID NO: 57 SEQ ID NO: 34 and SEQ ID NO: 58, SEQ ID NO: 35 and SEQ ID NO: 59, SEQ ID NO: 36 and SEQ ID NO: 60, SEQ ID NO: 37 and SEQ ID NO: 61, SEQ ID NO: 38 and SEQ ID NO: 62, SEQ ID NO: 39 and SEQ ID NO: 63, SEQ ID NO: 40 and SEQ ID NO: 64, SEQ ID NO: 41 and SEQ ID NO: 65, SEQ ID NO: 42 and SEQ ID NO: 66, SEQ ID NO: 43 and SEQ ID NO: 67, SEQ ID NO: 44 and SEQ ID NO: 68, SEQ ID NO: 45 and SEQ ID NO: 69, SEQ ID NO: 46 and SEQ ID NO: 70, SEQ ID NO: 47 and SEQ ID NO: 71, SEQ ID NO: 48 and SEQ ID NO: 72, SEQ ID NO: 49 and SEQ ID NO: 73, SEQ ID NO: 50 and SEQ ID NO: 74, SEQ ID NO: 51 and SEQ ID NO: 75, SEQ ID NO: 52 and SEQ ID NO: 76; SEQ ID NO: 53 and SEQ ID NO: 77 (Table 3).

The kits of the invention can also comprise one or more control nucleic acids or reference nucleic acids, such as nucleic acids comprising a CARD4 intronic sequence. For example, a kit can comprise primers for amplifying a polymorphic region of a CARD4 gene and a control DNA corresponding to such an amplified DNA and having the nucleotide sequence of a specific allelic variant. Thus, direct comparison can be performed between the DNA amplified from a patient and the DNA having the nucleotide sequence of a specific allelic variant. In one embodiment, the control nucleic acid comprises at least a portion of a CARD4 gene of an individual who does not have an inflammatory or allergic, or apoptotic disease, or a disease or disorder associated with an aberrant CARD4 activity.

Yet other kits of the invention comprise at least one reagent necessary to perform the assay. For example, the kit can comprise an enzyme. Alternatively the kit can comprise a buffer or any other necessary reagent.

The present invention is further illustrated by the following examples which should not be construed as limiting in any way. The contents of all cited references (including, without limitation, literature references, issued patents, published patent applications as well as the Figures, Tables, and database references) as cited throughout this application are hereby expressly incorporated by reference. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed. (1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLES

### Example 1

Determination of the Genomic Structure of the CARD4 Gene

This example describes the determination of the genomic structure of the CARD4 gene. Identification of the various functional regions of the gene, including, but not limited to, 5′ and 3′ untranslated regions (UTR) and intron/exon boundaries was necessary for subsequent variant detection experiments. Two sequence comparison software applications were used to elucidate genomic structure of the CARD4 gene. Both applications involve the comparison of the cDNA sequence of the CARD4 gene to the sequence of large genomic DNA clones (bacterial artificial chromosomes or BACs) that encode the CARD4 gene. One application is the Basic Local Alignment Search Tool™ or BLAST™ (Altschul et al. (1990) J. Mol. Biol. 215(3):403-410; Altschul et al. (1993) Nature Genetics 3:266-272; Altschul et al. (1997) Nuc. Acids Res. 25:3389-3402; Karlin and Altschul (1990) Proc. Natl. Acad. Sci. USA 87:2267-2268). The other application is Sequencher 3.1.1 (Gene Codes Corporation). Although based on different algorithms, these applications aid in the determination of genomic structure by the same principle.

These applications allow the comparison of two or more different nucleotide sequences (in the case of BLAST™, amino acid sequences can also be aligned) and identification or alignment of regions of high similarity or identity. Aligning cDNA sequence of the CARD4 gene with the corresponding BAC sequences with either tool allows visualization of regions of identity between the transcribed sequence and the sequence of the genomic region from which it was derived. Regions of sequence identity between the two sources are exonic regions and can be confirmed by the presence of known conserved splice elements flanking them. Regions of sequence contained in the BAC sequences but not in the cDNA sequence are considered to be either intronic sequence or lay outside of the 5′ and 3′ boundaries of the gene.

Table 2 provides the sequences at the boundries of the exons in GI 4156149. Thus, for each exon, Table 2 provides: the exon number (column 1), the GI number of the reference sequence (column 2), the 3′ sequence of the intron preceding the indicated exon (column 3) and its SEQ ID NO (column 4), the 5′ sequence of the exon (column 5) and its SEQ ID NO (column 6), the 3′ sequence of the indicated exon (column 7) and its SEQ ID NO (column 8), the 5′ sequence of the subsequent intron (column 9) and its SEQ ID NO (column 10), and the nucleotide numbers of the endpoints of the indicated exon (column 11).

Once the genomic structure of the CARD4 gene, e.g., introni/exon boundaries, was determined, PCR primers were designed for use with variant nucleotide detection experiments. Exonic sequences (including intron/exon junctions), 5′ UTR, 3′ UTR and approximately of 1 kbp upstream of the transcription start site were scanned for variant nucleotide detection as described in Examples 2, 3, 4, and 5.

### Example 2

Identification of Primer Pairs to Isolate Intronic, Exonic, and 5′ Upstream Regulatory Element Sequences for Detection of Polymorphisms and Mutations

Multiple pairs of primers were synthesized in order to amplify each of the exons or portions thereof and adjacent intronic regions. Genomic DNA from a human subject was subjected to PCR in 25 μl reactions (1× PCR Amplitaq polymerase buffer, 0.1 mM dNTPs, 0.8 μM 5′ primer, 0.8 μM 3′ primer, 0.75 units of Amplitaq polymerase, 50 ng genomic DNA) using each of the above described pairs of primers under the following cycle conditions: 94° C. for 2 min, 35×[94° C. for 40 sec, 57° C. for 30 sec, 72° C for 1 min], 72° C. for 5 min, 4° C. hold. The resulting PCR products were analyzed on a 2% agarose gel. The identity of the PCR product was confirmed by digestion with a restriction enzyme and subsequent agarose electrophoresis. Twenty-two pairs of oligomers were chosen to serve as PCR primers to amplify regions containing each of the 11 coding exons of the human CARD4 gene and two pairs of primers were chosen to serve as PCR primers to amplify the 5′ and 3′ untranscribed regions. The nucleotide sequences of the forward and reverse primers are indicated in Table 3, as well as the SEQ ID NOs for each primer and the location of the primers (e.g., exon, 3′ UTR, or 5′ UTR) within the CARD4 gene. The expected sizes of the PCR products are also set forth in Table 3. In addition, primer pair ID numbers corresponding to those listed in Table 1 are included where there was a polymorphism identified with a particular primer pair. Thus, polymorphisms CARD424a, CARD424b, and CARD424c in Table 1 were all identified with primer pair CARD-4-24 in Table 3.

### Example 3

Detection of Polymorphic Regions in the Human CARD4 Gene by DHPLC

This example describes the use of denaturing high performance liquid chromatography (DHPLC) for the identification of DNA sequence variations.

DNA samples for these experiments were obtained from a population of 96 individuals to be used for polymorphism discovery. These 96 DNA samples included DNA samples from a population of 28 North American Caucasian individuals, 28 African American individuals, and 28 individuals from throughout the Anhui province in East Central China. Furthermore, the 28 DNA samples from the Chinese individuals are from unrelated control subjects, obtained at random, and have been assessed for a number of traits related to asthma and allergies. Among the primary variables of interest are lung function , a response to “skin-prick” tests, physician's diagnosis of asthma, total serum IgE, and peripheral blood eosinophils. The peripheral blood eosinophil count was measured using the Coulter counter technique as described in Barnard et al. (1989) Clin Lab Haematol 11(3):255-66, and is expressed in terms of eosinophils per microliter. DHPLC uses reverse-phase ion-pairing chromatography to detect the heteroduplexes that are generated during amplification of PCR fragments from individuals who are heterozygous at a particular nucleotide locus within that fragment (Oefner and Underhill (1995) Am. J. Human Gen. 57:Suppl. A266).

Generally, the analysis was carried out as described in O'Donovan et al. ((1998) Genomics 52:44-49). PCR products having product sizes ranging from about 150-400 bp were generated using the primers and PCR conditions described in Example 2. Two PCR reactions were pooled together for DHPLC analysis (4 μl of each reaction for a total of 8 μl per sample). DHPLC was performed on a DHPLC system purchased from Transgenomic, Inc. The gradient was created by mixing buffers A (0.1M TEAA) and B (0.1M TEAA, 25% Acetontitrile). WAVEmaker™ software was utilized to predict a melting temperature and calculate a buffer gradient for mutation analysis of a given DNA sequence. The resulting chromatograms were analyzed to identify base pair alterations or deletions based on specific chromatographic profiles.

### Example 4

Detection of Polymorphic Regions in the Human CARD4 Gene by SSCP

Genomic DNA from each of the 96 individuals described in Example 3 was subjected to PCR in 25 μl reactions (1X PCR Amplitaq polymerase buffer, 0.1 mM dNTPs, 0.8 μM 5′ primer, 0.8 μM 3′ primer, 0.75 units of Amplitaq polymerase, 50 ng genomic DNA) using each of the above described pairs of primers under the following cycle conditions: 94° C. for 2 min, 35×[94° C. for 40 sec, 57° C. for 30 sec, 72° C. for 1 min], 72° C. 5 min, 4° C. hold. The expected sizes of the PCR products, are also indicated in Table 3.

The amplified genomic DNA fragments were then analyzed by SSCP (Orita et al. (1989) PNAS USA 86:2766, see also Cotton (1993) Mutat Res 285:125-144; and Hayashi (1992) Genet Anal Tech Appl 9:73-79). From each 25 μl PCR reaction, 3 μl was taken and added to 7 μl of loading buffer. The mixture was heated to 94° C. for 5 min and then immediately cooled in a slurry of ice-water. 3-4 μl were then loaded on a 10% polyacrylamide gel either with 10% glycerol or without 10% glycerol, and then subjected to electrophoresis either overnight at 4 Watts at room temperature, overnight at 4 Watts at 4° C. (for amplifying a 5′ upstream regulatory element), or for 5 hours at 20 Watts at 4° C. The secondary structure of single-stranded nucleic acids varies according to sequence, thus allowing the detection of small differences in nucleic acid sequence between similar nucleic acids. At the end of the electrophoretic period, the DNA was analyzed by gently overlaying a mixture of dyes onto the gel (1× the manufacturer's recommended concentration of SYBR Green I™ and SYBR Green II™ in 0.5× TBE buffer (Molecular Probes™)) for 5 min, followed by rinsing in distilled water and detection in a Fluoroimager 575™ (Molecular Dynamics™).

### Example 5

Identification of Polymorphic Regions in the Human CARD4 Gene by Direct Sequencing of PCR Products

To determine the sequences of the polymorphisms identified, the regions containing the polymorphisms were reamplified using the aforementioned primers. The genomic DNA from the subjects was subjected to PCR in 50 μl reactions (1× PCR Amplitaq polymerase buffer, 0.1 mM dNTPs, 0.8 μM 5′ primer, 0.8 μM 3′ primer, 0.75 units of Amplitaq polymerase, 50 ng genomic DNA) using each of the above described pairs of primers under the following cycle conditions: 94° C. for 2 min, 35×[94° C. for 40 sec, 57° C. for 30 sec, 72° C. for 1min], 72° C. 5 min, 4° C. hold. The newly amplified products were then purified using the Qiagen Qiaquick™ PCR purification kit according to the manufacturer's protocol, and subjected to sequencing using the aforementioned primers which were utilized for amplification.

Table 1 contains a “Polymorphism ID No.” in column 1, which is used herein to identify each individual CARD4 polymorphism. The nucleotide sequence flanking each polymorphism is provided in column 3, in which the polymorphic residue(s), having the wild-type or reference nucleotide, is indicated in lower-case letters. There are 10 nucleotides flanking the polymorphic nucleotide residue (i.e., 10 nucleotides 5′ of the polymorphism and 9 nucleotides 3′ of the polymorphism). Column 2 also indicates the sequence listing identifier number (SEQ ID NO.) of the sequence shown in Column 3 but with a variant nucleotide at the residue(s) shown in lower-case letter(s) or with a deletion of the sequences contained within the parenthesis. For example, SEQ ID NO: 4 contains an adenine (“g”) at the location indicated by the lower-case letter “t” in the corresponding sequence in column 3. Therefore, SEQ ID NO: 4 is identical to the corresponding sequence in column 2, except that the “t” (thymidine) residue is replaced by an “g” (guanine residue). Column 4 of Table 1 indicates the exon location for each polymorphisms located in an exon. Columns 5-7 of Table 1 indicate the reference codon (column 5), variant codon (column 6), and amino acid (column 7) for each silent polymorphism in the CARD4 coding sequence. Columns 8-10 of Table 1 indicate the reference codon (column 8), variant codon (column 9), and amino acid change (column 10) for each non-silent CARD4 polymorphism in the CARD4 coding sequence. Column 11 of Table 1 provides the nucleotide change (or insertion) and location (promoter, 3′ UTR, intron) for each non-coding region polymorphism. Column 12 of Table 1 provides the nucleotide position within either GenBank® GI 4156149 (genomic sequence; SEQ ID NO: 1) or GenBank® GI 11419372 (cDNA sequence; SEQ ID NO: 2) (or both) for each polymorphism. The sequence shown in Column 3 of Table 1 is the reverse complement of the genomic sequence within GenBank® GI 4156149. For example, the CARD406c polymorphism is a G to A change (shown in lowercase) according to the sequence shown in Column 3 of Table 1 (GCGGGACCCCgAGGAGGTGT). Column 12 of Table 1 indicates that this change corresponds to nucleotide 63787 of GenBank® GI 4156149. This corresponds to the sequence ACACCTCCTcGGGGTCCCGC within GenBank® GI 4156149 (SEQ ID NO: 1), which is the reverse complement of the sequence shown in Column 3 of Table 1. Thus, the G to A change in the sequence shown in Column 3 of Table 1 corresponds to a C to T change in the sequence shown in GenBank® GI 4156149 (SEQ ID NO: 1). Columns 13-16 of Table 1 provide the allele frequency for each polymorphism in the African American (AAC; column 13), North American Caucasian (1MR; column 14), Asian Chinese (ANQ; column 15), and Asian Chinese asthmatic (column 16) population studied.

The allele frequencies are based on a relatively small sample size and therefore should be regarded only very rough estimates. In some cases, no allele frequency is provided. Instead the number of heterozygous individuals is indicated.

### Example 6

Polymorphisms in the Human CARD4 Gene are Associated With Asthma

In order to identify genes and polymorphisms associated with asthma, a number of genes, including CARD4, were analyzed for evidence of association with asthma-related phenotypes in a sample of asthmatic pedigrees from Anqing, China.

Over 100 DNA polymorphisms within the genes were genotyped and analyzed. These include 101 SNPs, one insertion/deletion, and one microsatellite. A pedigree was included in the analysis if it contained at least one genotyped offspring with the asthma phenotype.

A standard methacholine challenge test was administered to assess the severity of the airway impairment. In a methacholine challenge, a patient inhales increasing dosages of methacholine until a dose that reduces forced expiratory volume by 20% (PD₂₀) is identified. The higher the dose (PD₂₀) required to cause a 20% decrease in forced expiratory volume, the less severe the impairment. PD₂₀ can be used to define phenotypes or patient classes. Based on this criterion, 361 pedigrees were analyzed using the PD₂₀<8 phenotype (most severe), 450 pedigrees were analyzed using the PD₂₀<20 phenotype, and 509 pedigrees were analyzed using the PD₂₀<50 phenotype (least severe). The average number of individuals per pedigree was 4.8. For each polymorphism, a minimum (min) Pval was determined within each of the three phenotypes (PD₂₀<8, PD₂₀<20, and PD₂₀<50). Based on this analysis five of the CARD4 polymorphisms listed in Table 1 exhibited statistically significant or near significant association with asthma in this population. The results of this analysis are shown in Table 4.

TABLE 4
CARD4 Polymorphisms Associated with Asthma

| Polymorphism | minimum Pval among | Location of Sequence |
|---|---|---|
| Name | three phenotypes | Change |
| CARD406c | min Pval = 0.005 | Coding Sequence |
| (SEQ ID | | G to A at nucleotide 925 SEQ |
| NO:11) | | ID NO:2 (E to K at amino acid |
| | | 266 of SEQ ID NO:3) |
| CARD402a | min Pval = 0.034 | Promoter region |
| (SEQ ID NO:6) | | A to G corresponds to T to C |
| | | at nucleotide 68258 of SEQ |
| | | ID NO:1 (reverse complement |
| | | of sequence in Table 1) |
| CARD410a | min Pval = 0.039 | 3′ UTR |
| (SEQ ID | | G to A at nucleotide 1849 of |
| NO:15) | | SEQ ID NO:2 |
| CARD405a | min Pval = 0.046 | Coding Sequence |
| (SEQ ID NO:8) | | C to T at nucleotide 612 of |
| | | SEQ ID NO:2 (silent change) |
| CARD403a | min Pval = 0.053 | Coding Sequence |
| (SEQ ID NO:7) | | C to G at nucleotide 285 of |
| | | SEQ ID NO:2 (silent change) |

Equivalents

Those skilled in the art will recognize, or be able to ascertain using no more than routine experimentation, many equivalents to the specific embodiments of the invention described herein. Such equivalents are intended to be encompassed by the following claims. A number of embodiments of the invention have been described. Nevertheless, it will be understood that various modifications may be made without departing from the spirit and scope of the invention.

| | | |
|---|---|---|
| SEQ | Silent Mutations | Coding Changes |

| | ID | | | Ref | Var | aa | Ref | Var | aa |
|---|---|---|---|---|---|---|---|---|---|
| Polymorphism ID | NO | Sequence | Exon | Codon | Codon | Change | Codon | Codon | Change |
| CARD401a | 4 | TAGAGATGGGIGTCTCACTA | | | | | | | |
| CARD401b | 5 | CTCCCAAAGgCTGGGATTA | | | | | | | |
| CARD402a | 6 | AGGTGGGGTGaGCTCTTTCT | | | | | | | |
| CARD403a | 7 | ACTTCTCGGCGAAGATGCG | 1 | GCC | GCG | A > A | | | |
| CARD405a | 8 | TCTACATGGAcACCATCATG | 3 | GAC | GAT | D > D | | | |
| CARO406a | 9 | CTGCTACAGCgGCTGCAGAG | 3 | | | | CGG | CAG | R > Q |
| CARD406b | 10 | GGGTCAAATTcTTCTTCCAC | 3 | | | | TTC | TTG | F > L |
| CARD406c | 11 | GCGGGACCCCgAGGAGGTGT | 3 | | | | GAG | AAG | E > K |
| CARD407a | 12 | ACCTGAGCCGcGTGCCTGAC | 3 | CGC | CGT | R > R | | | |
| CARD407b | 13 | GGCCCTGCAGgACCGCCTGC | 3 | | | | GAC | AAC | D > N |
| CARD408a | 14 | CGGAACACACgCAGCCCAGT | 3 | | | | CGC | CAC | R > H |
| CARD410a | 15 | GTGGTCCGGCgCGGGAAGAC | 3 | | | | | | |
| CARD411a | 16 | AGCCTGCGCCgCGTTGAGGT | 3 | | | | | | |
| CARD414a | 17 | TAGAAGGGGAIGGATGTATG | | | | | | | |
| CARD414b | 18 | GCTGTGTGTGIGGGGGGCGG | | | | | | | |
| CARD414c | 19 | TGTGTGGGGGg(insG)CGGGGCCTTG | | | | | | | |
| CARD418a | 20 | GCAAGTCCCTtAGGAAGGGG | | | | | | | |
| CARD418b | 21 | ATACAAGTTAIGTTCTTCTT | | | | | | | |
| CARD419a | 22 | GTGTGCGAGGaTCCTTAAGA | | | | | | | |
| CARD420a | 23 | ATGTGTCTTGcGAGCTGTTG | 11 | | | | | | |
| CARD420b | 24 | ATGTGAAGCTaGAGGAATAA | 11 | | | | | | |
| CARD420c | 25 | TATTATAATTa(insATTA)TTTTTATCT | 11 | | | | | | |
| CARD423a | 26 | GATGTTGGACa(insC)CCCGCCAGG | 11 | | | | | | |
| CARD424a | 27 | CCAGGGGTGTcACCAGTTTG | | | | | | | |
| CARD424b | 28 | TGATCGTTGAcAGGGCTCTG | | | | | | | |
| CARD424c | 29 | GACAGGAAGCgGCTGTGGGA | | | | | | | |

Variant Allele Frequency
by Screening

| Polymorphism ID | region | G#/residue | AAC | IMR | ANQ | M12AA |
|---|---|---|---|---|---|---|
| CARD401a | Promoter T>G | 4156149/68444 | | | | |
| CARD401b | Promoter G>A | 4156149/68365 | | | | |
| CARD402a | Promoter A>G | 4156149/68258 | | | | |
| CARD403a | | 4156149/67932;cDNA11419372/285 | 0.41 | 0.35 | 0.35 | 0.19 |
| CARD405a | | 4156149/64100;cDNA11419372/612 | 3 Hets | 3 Hets | 2 Hets | 2 Hets |
| CARO406a | | 4156149/63942;cDNA11419372/770 | | | | |
| CARD406b | | 4156149/63893;cDNA11419372/819 | | | | |
| CARD406c | | 4156149/63787;cDNA11419372/925 | | | | |
| CARD407a | | 4156149/63692:cDNA11419372/1020 | 0.11 | 0 | 0 | 0 |
| CARD407b | | 4156149/63469;cDNA11419372/1243 | 0.09 | 0 | 0 | 0.02 |
| CARD408a | | 4156149/63243;cDNA1419372/1469 | 0.11 | 0 | 0 | 0 |
| CARD410a | 3′UTR G > A | 4156149/62863;cDNA11419372/1849 | 0.2 | 0.35 | 0.28 | 0.24 |
| CARD411a | 3′UTR G > A | 4156149/62675;cDNA11419372/2037 | 1 Het | 0 Hets | 0 Hets | 0 Hets |
| CARD414a | Intron T > C | 4156149/57293 | | | | |
| CARD414b | Intron T > G | 4156149/57262 | | | | |
| CARD414c | Intron insG | 4156149/57256 | | | | |
| CARD418a | Intron T > C | 4156149/40687 | | | | |
| CARD418b | Intron T > C | 4156149/40639 | | | | |
| CARD419a | Intron A > G | 4156149/36926 | | | | |
| CARD420a | 3′UTR C > T | 4156149/36587:cDNA11419272/3208 | 0.3 | 0 | 0.05 | 0 |
| CARD420b | 3′UTR A > G | 4156149/38513:cDNA11419372/3282 | 0.3 | 0 | 0.05 | 0 |
| CARD420c | 3′UTR insATTA | 4156149/36529;cDNA11419372/3266 | 0.03 | 0 | 0 | 0 |
| CARD423a | 3′UTR InsC | 4156149/35808;cDNA11419372/3987 | 0.1 | 0 | 0.05 | 0 |
| CARD424a | 3′UTR C > A | 4156149/3552 | | | | |
| CARD424b | 3′UTR C > T | 4156149/35534 | | | | |
| CARD424c | 3′UTR G > A | 4156149/35502 | | | | |

!? ? ? SEQ ID? ? SEQ ID? ? SEQ ID? ? SEQ ID? ? ? !Exon#? GI#? intron seq? NO? exon seq? NO? exon seq? NO? intron seq? NO? residue#(5′ to 3′)

| | | | | | | | | | | |
|---|---|---|---|---|---|---|---|---|---|---|
| Exon 1 | 4156149 | TGTCATTGAT | 78 | CTCTTCAGGG | 89 | GCCTGACAAG | 100 | TGCCCCGG | 111 | 67887-68216 |
| Exon 2 | 4156149 | TCACCTCCAG | 79 | GTCCGCAAAA | 90 | ACTGACCCAG | 101 | GTAGGAGTC | 112 | 66303-66477 |
| Exon 3 | 4156149 | CTCCCTCCAG | 80 | TGAGCAGGTA | 91 | CTGTTCTCAG | 102 | GTGAGGCTG | 113 | 62384-64206 |
| Exon 4 | 4156149 | TTTATTCAG | 81 | ACTCAGCGTA | 92 | CCTATTTGGG | 103 | GTATGTCTTT | 114 | 59466-59549 |
| Exon 5 | 4156149 | TCTCTTGAAG | 82 | TTTATACAAC | 93 | CGCATCTTT | 104 | GTAAGTGGG | 115 | 58218-58135 |
| Exon 6 | 4156149 | CTCTCTGCAG | 83 | ACTGGGAAAA | 94 | CTGAGGTTGG | 105 | GTGAGTAGAA | 116 | 57392-57309 |
| Exon 7 | 4156149 | CCTCCCTCAG | 84 | GATGTGGGGC | 95 | CCACCCTGAG | 106 | GTAACTGTGG | 117 | 48824-48741 |
| Exon 8 | 4156149 | TCCTTCCTAG | 85 | TCTTGCGTCC | 96 | AAATACTGTG | 107 | GTAATAGCTC | 118 | 47166-47249 |
| Exon 9 | 4156149 | TTTTCCTTAG | 86 | GCTGACCCAA | 97 | AGCATTTATG | 108 | GTAACTCAGA | 119 | 44264-44347 |
| Exon 10 | 4156149 | CTTCTTTCAG | 87 | GCTTATCCAG | 98 | CAGAGATTTG | 109 | GTMGATCCC | 120 | 40625-40542 |
| Exon 11 | 4156149 | TTTTCAACAG | 88 | CCTAAATGGA | 99 | CTTGTCCTCT | 110 | TGCCAGCTCA | 121 | 36878-35735 |

!ID? pair? forward? SEQ ID NO? reverse? SEQ ID NO? location? size

| | | | | | | | |
|---|---|---|---|---|---|---|---|
| CARD4-1 | 1F/1R | GATCATCGTTCACTGCAGCCTTG | 30 | CACTGGTTGTGGGGTTTCTCTAG | 54 | 5′ | 301 |
| CARD4-2 | 2F/2R | CTTGCCTGGCCAGATAAAGGTC | 31 | GTAGCAAGCGGCTACTTTTCGC | 55 | 1 | 247 |
| CARD4-3 | 3F/3R | ATTGCCCTTCTGCTGAGAGGAC | 32 | TCCACACAATGCCATGCCCG | 56 | 1 | 312 |
| CARD4-4 | 4F/4R | AGGATGCCCCTCATGCCAGC | 33 | TGGTTGCACTGGTGCCTGCG | 57 | 2 | 267 |
| CARD4-5 | 5F/5R | CTCCTCACCCTGCTGCTGTG | 34 | TAGCAGCATGGACTTGCCCAC | 58 | 3 | 302 |
| CARD4-6 | 6F/6R | TGAGCAGGGTGAGACCATCTTC | 35 | AGTCCGAGTGCAGCTCGTCC | 59 | 3 | 302 |
| CARD4-7 | 7F/7R | TGGCCCTCTTCACCTTCGATG | 36 | GCACAGGCTGCAGAGGTTGG | 60 | 3 | 332 |
| CARD4-8 | 8F/8R | TGCAGGACCGCCTGCTGAG | 37 | ACAAAGAGGCTGTTCTCCATGCC | 61 | 3 | 322 |
| CARD4-9 | 9F/9R | AGTGGAGACCCTCCACGCC | 38 | AGCAGGACGTGGTCGCTGC | 62 | 3 | 327 |
| CARD4-10 | 10F/10R | CTGCTCAGGTTCTVCCAGGAG 39 | AACGTGGGCATGGCCTGCAC | 63 | 3 | 338 | |
| CARD4-11 | 11F/11R | ACACCTGT1T1CCAGCCTGCG | 40 | ATFCCCGATGCCCTCCGAGC | 64 | 3 | 403 |
| CARD4-12 | 12F/12R | ATAATGAGTGCCTGCCCTGACTC | 41 | GAATGTGAATTCCCTGCAGCTCTG | 65 | 4 | 202 |
| CARD4-13 | 13F/13R | TGACATCAGGAGCCAGAAAGTCTC | 42 | GGACAAGTGGAAGGCTTTAGGATG | 66 | 5 | 244 |
| CARD4-14 | 14F/14R | CACATTGGAGCCCCTGCATAG | 43 | AGTGGTCCTTCTGGTGTACTGATG | 67 | 6 | 238 |
| CARD4-15 | 15F/15R | ACTGGGCACAAACATCTGCCTG | 44 | TATTAGGAGCACGAATCACCCTTCC | 68 | 7 | 294 |
| CARD4-16 | 16F/16R | TCCCAGCTGTTCTGATGTTGAAGC | 45 | TGGACGACAAAGCAAGACCCTATC | 69 | 8 | 230 |
| CARD4-17 | 17F/17R | AGCTCCCGCTCCTGTGAACTC | 46 | TAGAATTAGGTGGTGATGGTTGCA | 70 | 9 | 304 |
| CARD4-18 | 18F/18R | AGCCAGATACAAAGTCACATGGAC | 47 | ACAGTCAGTGGTGGTGAGTAAACA | 71 | 10 | 290 |
| CARD4-19 | 19F/19R | ACGAGTGTGTGCTATGAACACACC | 48 | ACAGGAAGCTGGCTTGCATCATG | 72 | 11 | 316 |
| CARD4-20 | 20F/20R | TGCGCAGGCGGGACTATCAG | 49 | AGAGCGGTGACCAACTCGCTC | 73 | 11 | 293 |
| CARD4-21 | 21F/21R | AGCCTCACCTCAUCCAACACC | 50 | TGCCAGCAGACAGTGAGACTC | 74 | 11 | 331 |
| CARD4-22 | 22F/22R | ATGTCAAGGAAAGGATGCACCAC | 51 | TCAGCTGCTTGGGAGGGAGG | 75 | 11 | 337 |
| CARD4-23 | 23F/23R | TGTCTCCCTTACCTCGTGAAGAG | 52 | TGAGTGCTTACCAAGCCAAGTCC | 76 | 11 | 285 |
| CARD4-24 | 24F/24R | ACTGAAAACACTCTTACGGGUGA | 53 | ACAAGTTATTTCCTCTGAGAAGCCA | 77 | 3′ | 284 |

What is claimed is:

1. An isolated nucleic acid molecule comprising an allelic variant of a CARD4 gene, wherein the allelic variant comprises a nucleotide sequence selected from the group consisting of those set forth in SEQ ID NOs: 4-29, or the complement thereof.

2. The isolated nucleic acid molecule of claim 1, wherein the allelic variant further comprises two or more nucleotide sequences selected from the group consisting of those set forth in SEQ ID NOs: 4-29, or the complement thereof.

3. The isolated nucleic acid molecule of claim 1 wherein the allelic variant comprises a nucleotide sequence selected from the group consisting of those set forth in SEQ ID NOs: 6-8, 11 and 15, or the complement thereof.

4. A kit comprising a probe or primer which is capable of selectively hybridizing to the nucleic acid molecule of claim 1 under stringent conditions, the probe or primer not being capable of selectively hybridizing under stringent to a nucleic acid molecule consisting of SEQ ID NO: 1 or SEQ ID NO: 2.

5. The kit of claim 4, wherein the probe or primer comprises a nucleotide sequence from about 15 to about 30 nucleotides.

6. The kit of claim 5, wherein the probe or primer comprises a nucleotide sequence selected from the group consisting of nucleic acids having a nucleotide sequence set forth in SEQ ID NOs: 4-29.

7. The kit of claim 6 wherein the probe or primer comprises a nucleotide sequence selected from the group consisting of nucleic acids having a nucleotide sequence set forth in SEQ ID NOs: 6-8, 11 and 15.

8. The kit of claim 5, wherein the probe or primer is labeled

9. A method for determining whether an patient will be responsive to treatment with a CARD4 modulator, comprising
a) obtaining a nucleic acid sample from the patient;
b) determining the presence of an allelic variant which differs from the reference sequence set forth in SEQ ID NO: 1 or SEQ ID NO: 2; and
c) determining whether the patient will be responsive to treatment with a CARD4 modulator based on the presence of an allelic variant which differs from the reference sequence set forth in SEQ ID NO: 1 or SEQ ID NO: 2, wherein the allelic variant comprises one or more nucleotide sequences selected from the group consisting of those set forth in SEQ ID NOs: 4-29.

10. A method for determining whether an patient has a more or less severe phenotype of an apoptotic, inflammatory or allergic disorder, comprising
a) obtaining a nucleic acid sample from the patient;
b) determining the presence of an allelic variant which differs from the reference sequence set forth in SEQ ID NO: 1 or SEQ ID NO: 2; and
c) determining whether the patient has a more or less severe phenotype of an apoptotic, inflammatory or allergic disorder based on the presence of an allelic variant which differs from the reference sequence set forth in SEQ ID NO: 1 or SEQ ID NO: 2, wherein the allelic variant comprises one or more nucleotide sequences selected from the group consisting of those set forth in SEQ ID NOs: 4-29.

11. A method for selecting the appropriate drug to administer to a patient who has an apoptotic, inflammatory or allergic disorder, comprising
a) obtaining a nucleic acid sample from the patient;
b) determining the presence of an allelic variant which differs from the reference sequence set forth in SEQ ID NO: 1 or SEQ ID NO: 2; and
c) selecting the appropriate drug to administer to a patient who has an apoptotic, inflammatory or allergic disorder based on the presence of an allelic variant which differs from the reference sequence set forth in SEQ ID NO: 1 or SEQ ID NO: 2, wherein the allelic variant comprises one or more nucleotide sequences selected from the group consisting of those set forth in SEQ ID NOs: 4-29.

12. The method of any of claims 9-11 wherein the allelic variant comprises a nucleotide sequence selected from the group consisting of those set forth in SEQ ID NOs: 6-8, 11 and 15.

13. The method of claim 11, wherein the drug is a CARD4 inhibitor.

14. A method of identifying a patient who is a candidate for effective treatment with a CARD4 inhibitor comprising the steps of:
a) obtaining a nucleic acid sample from the patient;
b) determining the presence of an allelic variant which differs from the reference sequence set forth in SEQ ID NO: 1 or SEQ ID NO: 2; and
c) identifying a patient who is a candidate for effective treatment with a CARD4 inhibitor based on the presence of an allelic variant which differs from the reference sequence set forth in SEQ ID NOs: 1 or SEQ ID NO: 2, wherein the allelic variant comprises one or more nucleotide sequences selected from the group consisting of those set forth in SEQ ID NOs: 4-29, or the complement thereof.

15. The method of claim 14, wherein the patient has an apoptotic, inflammatory or allergic disorder.

16. The method of claim 14 wherein the wherein the allelic variant comprises one or more nucleotide sequences selected from the group consisting of those set forth in SEQ ID NOs: 6-8, 11 and 15.

17. A method for determining the identity of an allelic variant of a CARD4 gene in a nucleic acid obtained from a patient, wherein the sample comprises a CARD4 gene sequence, comprising contacting a sample nucleic acid from the patient with a probe having a sequence which is complementary to a CARD4 gene sequence having a polymorphism listed in Table 1, thereby determining the identity of the allelic variant.

18. The method of claim 17, wherein determining the identity of the allelic variant is carried out by single-stranded conformation polymorphism.

19. The method of claim 17, wherein determining the identity of the allelic variant is carried out by allele specific hybridization.

20. The method of claim 17, wherein determining the identity of the allelic variant is carried out by primer specific extension.

21. The method of claim 17, wherein determining the identity of the allelic variant is carried out by an oligonucleotide ligation assay.

22. A method for determining whether an asthma patient will be responsive to treatment with a CARD4 modulator, comprising
a) obtaining a nucleic acid sample from the patient;
b) determining the presence of an allelic variant which differs from the reference sequence set forth in SEQ ID NO: 1 or SEQ ID NO: 2; and
c) determining whether the asthma patient will be responsive to treatment with a CARD4 modulator based on the presence of an allelic variant which differs from the reference sequence set forth in SEQ ID NO: 1 or SEQ ID NO: 2, wherein the allelic variant comprises one or more nucleotide sequences selected from the group consisting of those set forth in SEQ ID NOs: 4-29.

23. A method for determining whether an patient is suffering from or is susceptible to asthma, comprising
a) obtaining a nucleic acid sample from the patient;
b) determining the presence of an allelic variant which differs from the reference sequence set forth in SEQ ID NO: 1 or SEQ ID NO: 2; and
c) determining whether the patient is suffering from or is susceptible to asthma based on the presence of an allelic variant which differs from the reference sequence set forth in SEQ ID NO: 1 or SEQ ID NO: 2, wherein the allelic variant comprises one or more nucleotide sequences selected from the group consisting of those set forth in SEQ ID NOs: 4-29.

24. A method for selecting the appropriate drug to administer to a patient who has asthma, comprising
a) obtaining a nucleic acid sample from the patient;
b) determining the presence of an allelic variant which differs from the reference sequence set forth in SEQ ID NO: 1 or SEQ ID NO: 2; and
c) selecting the appropriate drug to administer to a patient who has asthma based on the presence of an allelic variant which differs from the reference sequence set forth in SEQ ID NO: 1 or SEQ ID NO: 2, wherein the allelic variant comprises one or more nucleotide sequences selected from the group consisting of those set forth in SEQ ID NOs: 4-29.

25. The method of any of claims 22-24 wherein the allelic variant comprises a nucleotide sequence selected from the group consisting of those set forth in SEQ ID NOs: 6-8, 11 and 15.

26. The method of claim 24, wherein the drug is a CARD4 inhibitor.

27. A method of identifying an asthma patient who is a candidate for effective treatment with a CARD4 modulator comprising the steps of:
a) obtaining a nucleic acid sample from the patient;
b) determining the presence of an allelic variant which differs from the reference sequence set forth in SEQ ID NO: 1 or SEQ ID NO: 2; and
c) identifying a asthma patient who is a candidate for effective treatment with a CARD4 modulator based on the presence of an allelic variant which differs from the reference sequence set forth in SEQ ID NOs: 1, or SEQ ID NO: 2, wherein the allelic variant comprises one or more nucleotide sequences selected from the group consisting of those set forth in SEQ ID NOs: 4-29, or the complement thereof.

28. The method of claim 27 wherein the wherein the allelic variant comprises one or more nucleotide sequences selected from the group consisting of those set forth in SEQ ID NOs: 6-8, 11 and 15.

29. A method for treating a patient having a apoptotic, inflammatory or allergic disorder comprising:
a) determining the presence of an allelic variant which differs from the reference sequence set forth in SEQ ID NO: 1 or SEQ ID NO: 2;
b) identifying a patient who is a candidate for effective treatment with a selected CARD4 modulator based on the presence of an allelic variant which differs from the reference sequence set forth in SEQ ID NOs: 1 or SEQ ID NO: 2, wherein the allelic variant comprises one or more nucleotide sequences selected from the group consisting of those set forth in SEQ ID NOs: 4-29; and
c) administering a CARD4 modulator to the patient identified as a candidate for effective treatment with a selected CARD4 modulator.

30. The method of claim 29 wherein the allelic variant is located in an exon.

31. The method of claim 29 wherein the allelic variant is located in an intron.

32. The method of claim 29 wherein the polymorphic region is located in a promoter region.

33. The method of claim 29 wherein the polymorphic region is located in a 3′ untranslated region.

34. The method of claim 29 wherein the allelic variant comprises one or more nucleotide sequences selected from the group consisting of those set forth in SEQ ID NOs: 6-8, 11 and 15.

35. A method for treating a patient having asthma comprising:
a) determining the presence of an allelic variant which differs from the reference sequence set forth in SEQ ID NO: 1 or SEQ ID NO: 2;
b) identifying an asthma patient who is a candidate for effective treatment with a selected CARD4 modulator based on the presence of an allelic variant which differs from the reference sequence set forth in SEQ ID NOs: 1 or SEQ ID NO: 2, wherein the allelic variant comprises one or more nucleotide sequences selected from the group consisting of those set forth in SEQ ID NOs: 4-29; and
c) administering a CARD4 modulator to the asthma patient identified as a candidate for effective treatment with a selected CARD4 modulator.

36. The method of claim 35 wherein the allelic variant is located in an exon.

37. The method of claim 35 wherein the allelic variant is located in an intron.

38. The method of claim 35 wherein the polymorphic region is located in a promoter region.

39. The method of claim 35 wherein the polymorphic region is located in a 3′ untranslated region.

40. The method of claim 35 wherein the allelic variant comprises one or more nucleotide sequences selected from the group consisting of those set forth in SEQ ID NOs: 6-8, 11 and 15.
